(19) United States
(12) Patent Application Publication
(10) Pub. No.: US 2004/0219530 A1
(43) Pub. Date: Nov. 4, 2004

(54) Array and uses thereof

(76) Inventors: Roland Brousseau, Montreal (CA); Josee Harel, Saint-Bruno (CA); Sadjia Bekal, Montreal (CA)

Correspondence Address: OGILVY RENAULT, 1981 MCGILL COLLEGE AVENUE, SUITE 1600, MONTREAL, QC, H3A2Y3, CA

(21) Appl. No.: 10/425,821

(22) Filed: Apr. 30, 2003

(51) Int. Cl. C12Q 1/68

(52) U.S. Cl. 435/6

(57) ABSTRACT

An array of nucleic acid probes is described for identifying and/or characterizing a pathotype of a microorganism. Methods are also described for detecting the presence of a microorganism in a sample, as well as determining its pathotype, using the array. Methods of assessing related infection and disease in a subject using the array are also described.

### FIELD OF THE INVENTION

The invention relates to an array and uses thereof and particulary relates to an array for pathotyping a microorganism and uses thereof.

### BACKGROUND OF THE INVENTION

A variety of pathogenic microorganisms exist, which pose a continued health threat. An example is the bacterium Escherichia coli, which is commonly found in the environment as well as in the digestive tracts of common animal species including humans. Individual strains within Escherichia coli (E. coli) can vary in pathogenicity from innocuous to highly lethal, as evidenced by incidents of its contamination of drinking water and outbreaks of so-called hamburger disease. The pathogenicity of a given E. coli depends on the presence or absence of virulence genes within its genome. These virulence genes are ideal targets for the determination of the pathogenicity potential of any given E. coli isolate.

Numerous molecular methods have been used for detecting and identifying pathogenic E. coli. However, these approaches suffer from a variety of limitations, the, most serious of which is related to the large variety of virulence factors distributed among the known pathotypes. Currently, there is no practical, cost-effective way to determine rapidly and simultaneously the presence or absence of this large set of these virulence genes within a given E. coli strain.

It would therefore be desirable to have improved methods and materials for the detection of pathogenic microorganisms, such as bacteria (e.g. E. coli) .

### SUMMARY OF THE INVENTION

The invention relates to a collection of probes, e.g. in an array format, and uses thereof.

Accordingly, in a first aspect, the invention provides an array comprising: a substrate; and a plurality of nucleic acid probes, each of the probes being bound to the substrate at a discrete location; the plurality of probes comprising a first probe for a first pathotype of a species of a microorganism and a second probe for a second pathotype of the species, wherein the first and second pathotypes are not identical. In an embodiment, the array comprises at least 103 distinct nucleic acid probes. In embodiments, each of the probes are independently greater than or equal to 20, 50 or 100 nucleotides in length. In an embodiment, the array comprises at least two probes for a single pathotype, wherein the two probes are not identical. In an embodiment, the array comprises a subarray, wherein the subarray comprises the at least two probes at adjacent discrete locations on the substrate.

In an embodiment, the plurality of probes comprises, first, second, third and fourth probes for respective first, second, third and fourth pathotypes of the species, wherein the first, second, third and fourth pathotypes are not identical. In a further embodiment, the plurality of probes comprises, first, second, third, fourth, fifth and sixth probes for respective first, second, third, fourth, fifth and sixth pathotypes of the species, wherein the first, second, third, fourth, fifth and sixth pathotypes are not identical. In yet a further embodiment, the plurality of probes comprises, first, second, third, fourth, fifth, sixth, seventh and eighth probes for respective first, second, third, fourth, fifth, sixth, seventh and eighth pathotypes of the species, wherein the first, second, third, fourth, fifth, sixth, seventh and eighth pathotypes are not identical.

In an embodiment, the probe is for a virulence gene or fragment thereof or a sequence substantially identical thereto, wherein the virulence gene is associated with pathogenicity of the microorganism.

In an embodiment, the microorganism is a bacterium, in a further embodiment, of the family Enterobacteriaceae, in a further embodiment, the bacterium is E. coli.

In an embodiment, the first and second pathotypes each independently comprise a pathotype selected from the group consisting of: enterotoxigenic E. coli (ETEC); enteropathogenic E. coli (EPEC); enterohemorrhagic E. coli (EHEC); enteroaggregative E. coli (EAEC); enteroinvasive E. coli (EIEC); uropathogenic strains (UPEC); E. coli strains involved in neonatal meningitis (MENEC); E. coli strains involved in septicemia (SEPEC); cell-detaching E. coli (CDEC); and diffusely adherent E. coli (DAEC).

In an embodiment, the first pathotype is selected from the group consisting of: enteroaggregative E. coli (EAEC); enteroinvasive E. coli (EIEC); E. coli strains involved in neonatal meningitis (MENEC); E. coli strains involved in septicemia (SEPEC); cell-detaching E. coli (CDEC); and diffusely adherent E. coli (DAEC).

In an embodiment, the virulence gene encodes a polypeptide of a class of proteins selected from the group consisting of toxins, adhesion factors, secretory system proteins, capsule antigens, somatic antigens, flagellar antigens, invasins, autotransporter proteins, and aerobactin system proteins. In an embodiment, the virulence gene is selected from the group consisting of afaBC3, afaE5, afaE7, afaD8, aggA, aggC, aida, bfpA, bmaE, cdt1, cdt2, cdt3, cfaI, clpG, cnf1, cnf2, cs1, cs3, cs31a, cvaC, derb122,eae, eaf, east1, ehxA, espA group I, espA group II, espA group III, espB group I, espB group II, espB group III, espC, espP, etpD, F17A, F17G, F18, F4, F41, F5, F6, fimA group I, fimA group II, fimH, fliC, focG, fyuA, hlyA, hlyC, ibe10, iha, invX, ipaC, iroN, irp1, irp2, iss, iucD, iutA, katP, kfiB, kpsMTII, kpsMTIII, 17095, leoA, IngA, lt, neuC, nfaE, ompA, ompT, paa, papAH, papC, papEF, papG group I, papG group II, papG group III, pai, rfbO9, rfbO101, rfbO111, rfbE O157, rfbE O157 H7, rfc O4, rtx, sfaDE, sfaA, stah, stap, stb, stx1, stx2, stxA I, stxA II, stxB I, stx B II, stxB III, tir group I, tir group II, tir group III, traT, and tsh genes. In an embodiment, the above-noted probe comprises a nucleic acid sequence selected from the group consisting of SEQ ID NO:1 to SEQ ID NO:102, or a fragment thereof, or a sequence substantially identical thereto.

In an embodiment, the substrate is selected from the group consisting of a porous support and a support having a non-porous surface. In embodiments the support is selected from the group consisting of a slide, chip, wafer, membrane, filter and sheet. In an embodiment, the slide comprises a coating capable of enhancing nucleic acid immobilization to the slide. In an embodiment, the probes are covalently attached to the substrate.

The invention further provides a method of detecting the presence of a microorganism in a sample, the method comprising: contacting the above-mentioned array with a sample nucleic acid of the sample; and detecting association of the sample nucleic acid to a probe on the array; wherein association of the sample nucleic acid with the probe is indicative that the sample comprises a microorganism from which the nucleic acid sequence of the probe is derived. In an embodiment, the sample nucleic acid comprises a label. In an embodiment, the label is a fluorescent dye (e.g. a cyanine, a fluorescein, a rhodamine and a polymethine dye derivative). In an embodiment, the method further comprises extracting the sample nucleic acid from the sample before contacting it with the array. In an embodiment, the sample nucleic acid is not amplified by PCR prior to contacting it with the array. In an embodiment, the method further comprises digesting the sample nucleic acid with a restriction enzyme to produce fragments of the sample nucleic acid prior to contacting with the array. In an embodiment, the fragments are of an average size of about 0.2 Kb to about 12 Kb. In an embodiment, the method further comprises labelling the sample nucleic acid prior to contacting it with the array. In an embodiment, the sample nucleic acid is selected from the group consisting of DNA and RNA.

In an embodiment, the above-mentioned sample is selected from the group consisting of environmental samples, biological samples and food. In an embodiment, the environmental samples are selected from the group consisting of water, air and soil. In an embodiment, the biological samples are selected from the group consisting of blood, urine, amniotic fluid, feces, tissues, cells, cell cultures and biological secretions, excretions and discharge.

In an embodiment, the method is further for determining a pathotype of a species of the microorganism, wherein the probe is for a pathotype of the species and wherein association of the sample nucleic acid with the probe is indicative that the microorganism is of the pathotype.

In an embodiment, the sample is a tissue, body fluid, secretion or excretion from a subject and the method is further for diagnosing an infection by the microorganism in the subject, wherein association of the nucleic acid with the probe is indicative that the subject is infected by the microorganism.

In an embodiment, the method is for diagnosing a condition related to infection by the microorganism in the subject, wherein the probe is for a pathotype of the species and wherein association of the sample nucleic acid with the probe is indicative that the microorganism is of the pathotype and that the subject suffers from a condition associated with the pathotype. In an embodiment, the condition is selected from the group consisting of: diarrhea, hemorrhagic colitis, hemolytic uremic syndrome, invasive intestinal infections, dysentery, urinary tract infections, neonatal meningitis and septicemia. In an embodiment, the subject is a mammal, in a further embodiment, a human.

The invention further provides a commercial package comprising the above-mentioned array together with instructions for: (a) detecting the presence of a microorganism in a sample; (b) determining the pathotype of a microorganism in a sample; (c) diagnosing an infection by a microorganism in a subject; (d) diagnosing a condition related to infection by a microorganism, in a subject; or (e) any combination of (a) to (d).

The invention further provides a use of the above-mentioned array for: (a) detecting the presence of a microorganism in a sample; (b) determining the pathotype of a microorganism in a sample; (c) diagnosing an infection by a microorganism in a subject; (d) diagnosing a condition related to infection by a microorganism, in a subject; or (e) any combination of (a) to (d).

The invention further provides a method of producing an array for pathotyping a microorganism in a sample, the method comprising: providing a plurality of nucleic acid probes, the plurality of probes comprising a first probe for a first pathotype of a species of the microorganism and a second probe for a second pathotype of the species, wherein the first and second probes are different; and applying each of the plurality of probes to a different discrete location of a substrate. In an embodiment, the method further comprises the step of crosslinking by exposure of the array to ultraviolet radiation. In an embodiment, the method further comprises heating the array subsequent to the crosslinking.

The invention further provides a method of producing an array for pathotyping a microorganism in a sample, the method comprising: selecting a plurality of nucleic acid probes, the plurality of probes comprising a first probe for a first pathotype of a species of the microorganism and a second probe for a second pathotype of the species, wherein the first and second probes are different; and synthesizing each of the plurality of probes at a different discrete location of a substrate.

### BRIEF DESCRIPTION OF THE DRAWINGS

FIG. 1: Print pattern of the E. coli pathotype microarray according to an embodiment of the invention. (A) Grouping of genes by category (B) Location of the individual genes.

FIG. 2: Detection of virulence genes and simultaneous identification of the pathotype of known E. coli strains after microarray hybridization with genomic DNA from (A) a nonpathogenic K-12 E. coli strain DH5α (B) an enterohemorrhagic strain EDL933 0157:H7 (C) an uropathogenic strain J96, 04:K6 and (D) an enterotoxigenic strain H-10407. Genomic DNA after HindIII/EcoRI digestion was labeled with Cy3. Labeled DNA (500 ng) was hybridized to the array overnight at 42° C., washed, dried and scanned. Boxed spots in Panel A represent the virulence genes present in K-12 E. coli strain DH5α (traT, fimA, fimH, ompA, ompT, iss, fliC). Boxed spots in Panels B, C and D indicate the pathotype-specific genes in the tested strains. Genes present in more than one pathotype (iss, irp2, fliC, ompT) or present in all the pathotypes (fimH, fimA, ompA) gave a positive signal. The horizontal bar indicates the color representation of fluorescent-signal intensity.

FIG. 3: Virulence potential analysis of E. coli strains isolated from clinical samples using a E. coli pathotype microarray according to an embodiment of the invention. (A) Hybridization of genomic DNA from an avian E. coli isolate Av01-4156 (B) Hybridization pattern obtained with genomic DNA from a bovine strain B00-4830 (C) Hybridization of genomic DNA from a human E. coli isolate H87-540. Labeled DNA (500 ng) was hybridized to the array overnight at 42° C. after which the slide was washed, dried and scanned. Boxed spots indicate the pathotype-specific genes: iucD, iron, traT and iutA in panel A, etpD, F5, stap, and traT in panel B, stx1, cdt2, cdt3, afaD8, bmaE, iucD, iroN, and iutA in Panel C. Positive signals were also obtained with genes present in more than one pathotype (espP, iss, ompT, fliC) and genes present in all the tested pathotypes (fimA, fimH, ompA).

FIG. 4: Detection of stx and cnf variant genes in clinical isolates of E. coli using a pathotype microarray according to an embodiment of the invention. The white boxes in Panel A outlines the stx genes hybridized with (1) the human strain H87-5406 and (2) the bovine strain B99-4297. The white boxes in Panel B outlines the cnf genes hybridized with (1) strain Ca01-E179 and (2) strain H87-5406. Labeled DNA (500 ng) was hybridized to an array overnight at 42° C. after which the slide was washed, dried and scanned.

FIG. 5: Use of an E. coli pathotype microarray according to an embodiment of the invention to identify the phylogenetic group of E. coli strains on the basis of their hybridization pattern with the attaching and effacing gene probes (A) print pattern of espA, espB and tir probes on the pathotype microarray with the homology percentages between each immobilized probe (B) detection of espA3, espB2 and tir3 in the human EPEC strain E2348/69 (C) hybridization pattern obtained with genomic DNA from the animal EPEC strain P86-1390 (espA1, espB3 and tir1 (D) detection of espA2, espB1 and tir2 in the EHEC strain EDL933. The positive hybridization results obtained with espA, espB and tir probes are outlined in white boxes.

FIG. 6: Schematic of virulence gene DNA microarray for Escherichia coli according to an embodiment of the invention. The number and alignment of DNA probes within sub-arrays and of sub-arrays within the microarray can vary as required. The embodiment illustrated depicts a subarray of 12 different gene probes (g1-g12), each being spotted twice. The 24 subarrays shown would represent 24×12=288 distinct virulence genes.

FIG. 7: Schematic representation of a method of use of a virulence gene microarray according to an embodiment of the invention.

### DETAILED DESCRIPTION OF THE INVENTION

The present invention provides products and methods for the detection and characterization of microorganisms, such as bacteria, (e.g. of the family Enterobacteriaceae) such as E. coli. The products and methods of the invention can be used to detect the presence of such a microorganism in a sample (e.g. a biological or environmental sample). Further, such products and methods can be used to characterize such a microorganism, e.g. determining/characterizing its pathotype.

Pathogenic E. coli are responsible for three main types of clinical infections (a) enteric/diarrheal disease (b) urinary tract infections and (c) sepsis/meningitis. On the basis of their distinct virulence properties and clinical symptoms of the host, pathogenic E. coli are divided into numerous categories or pathotypes. The diarrheagenic E. coli include (i) enterotoxigenic E. coli (ETEC) associated with traveller's diarrhea and porcine and bovine diarrhea, (ii) enteropathogenic E. coli (EPEC) causing diarrhea in children and animals, (iii) enterohemorrhagic E. coli (EHEC) associated with hemorrhagic colitis and hemolytic uremic syndrome in humans, (iv) enteroaggregative E. coli (EAEC) associated with persistent diarrhea in humans, and (v) enteroinvasive E. coli (EIEC) involved in invasive intestinal infections, watery diarrhea and dysentery in humans and animals (71). Extra-intestinal infections are caused by three separate E. coli pathotypes (i) uropathogenic strains (UPEC) that cause urinary tract infections in humans, dogs and cats (8, 36, 87) (ii) strains involved in neonatal meningitis (MENEC) (87) and (iii) strains that cause septicemia in humans and animals (SEPEC) (25, 41, 66, 87).

Numerous bioassays and molecular methods have been developed for the detection of genes involved in pathogenic E. coli virulence mechanisms. However, the sheer numbers of known virulence factors have made this a daunting task. As described herein, microarray technology offers the most rapid and practical tool to detect the presence or absence of a large set of virulence genes simultaneously within a given E. coli strain. Prior to applicants' findings herein, only a few studies have reported the use of microarrays as a diagnostic tool (16, 18, 19, 63, 70). Described herein is a new approach for detection of a large number of virulence factors present in E. coli strains and the subsequent determination of the strain's pathotype. As described herein, nucleic acid sequences derived from most known virulence factors including associated-virulence genes were amplified by PCR and immobilized onto glass slides to create a virulence DNA microarray chip. Probing this virulence gene microarray with labeled genomic E. coli DNA, the virulence pattern of a given strain can be assessed and its pathotype determined in a single experiment.

As a practical example in support of this invention, an E. coli virulence factor microarray was designed and tested. It was of course recognized that applications of this microarray reach far into human health, drinking water and environmental research.

According to another aspect of the invention, a method is provided for analyzing a given liquid culture or colony of bacteria simultaneously for the presence of a number of these virulence genes in the same experiment.

In embodiments, an array of virulence genes may be used by reference laboratories involved in public or veterinary health. A simplified format of the microarray focusing on a few key virulence genes could find a broader market in routine medical or veterinary microbiological laboratory work.

Other types of virulence genes may be represented on such an array for a variety of applications. For example, the armed forces may be interested in implementing this type technology for detection and/or identification of biological warfare agents.

The invention thus relates to products and methods which enable the parallel analysis in respect of a plurality of pathotypes of a microorganism(s), via the use of a collection of a plurality of nucleic acid probes derived from virulence genes of the microorganism(s), the collection corresponding to a plurality of pathotypes of the microorganism(s). In embodiments, the plurality of pathotypes may comprise at least 2, 3, 4, 5, 6, 7, 8, 9 or 10 pathotypes.

Accordingly, in an aspect, the invention relates to a collection comprising a plurality of probes, the probes being derived from genetic/protein (e.g. a virulence gene) material/information from a microorganism and correspond to a plurality of pathotypes of the microorganism. In an embodiment, the probes comprise a nucleic acid sequence derived from a microorganism or a sequence substantially identical thereto. In an embodiment, the collection can represent more than one microorganism.

“Pathotype” as used herein refers to the classification of a particular strain of a microorganism by virtue of the pathogenic phenotype it may manifest when it infects a subject. A plurality of strains may thus be grouped in the same pathotype if the strains are capable of resulting in the same phenotypic manifestation (e.g. disease symptoms) when they infect a subject. In the case of E. coli, for example, pathotypes may include those associated with intestinal and extraintestinal conditions. Such pathotypes include but are not limited to ETEC, EPEC, EHEC, EAEC, EIEC, UPEC, MENEC, SEPEC, CDEC and DAEC noted herein. As described herein, a pathotype may be identified and/or characterized using a probe based on a virulence gene associated with the pathotype, in a particular microorganism. “Virulence gene” as used herein refers to a nucleic acid sequence of a microorganism, the presence and/or expression of which correlates with the pathogenicity of the microorganism. In the case of bacteria, such virulence genes may in an embodiment comprise chromosomal genes (i.e. derived from a bacterial chromosome), or in a further embodiment comprise a non-chromosomal gene (i.e. derived from a bacterial non-chromosomal nucleic acid source, such as a plasmid) . In the case of E. coli, examples of virulence genes and classes of polypeptides encoded by such genes are described below. Virulence genes for a variety of pathogenic microorganisms are known in the art.

Two probes which are “not identical” as used herein denotes two probes that have at least one structural difference. The difference may for example comprise an addition, deletion or substitution of one or more nucleotides or a rearrangement within its nucleotide sequence. Two pathotypes which are “not identical” as described herein denotes two classifications of pathogenic microorganisms that are sufficiently different to result in recognizably different pathogenic phenotypic manifestations when infecting a subject.

In an embodiment, the above-noted collection is in the form of an array, whereby the probes are bound to different, discrete locations of a substrate. The length of the probes may be variable, e.g. at least 20, 50, 100, 500, 1000 or 2000 nucleotides in length. High density nucleic acid probe arrays, also referred to as “microarrays,” may for example be used to detect and/or monitor the expression of a large number of genes, or for detecting sequence variations, mutations and polymorphisms. Microfabricated arrays of large numbers of oligonucleotide probes, (variously described as “biological chips”, “gene chips”, or “DNA chips”), allow the simultaneous nucleic acid hybridization analysis of a target DNA molecule with a very large number of oligonucleotide probes. In one aspect, the invention provides biological assays using such high density nucleic acid or protein probe arrays. For the purpose of such arrays, “nucleic acids” may include any polymer or oligomer of nucleosides or nucleotides (polynucleotides or oligonucleotidies), which include pyrimidine and purine bases, preferably cytosine, thymine, and uracil, and adenine and guanine, respectively. Polymers or oligomers of deoxyribonucleotides or ribonucleotides may be used, which may contain naturally occurring or modified bases, and which may contain normal internucleotide bonds or modified (e.g. peptide) bonds. A variety of methods are known for making and using microarrays, as for example disclosed in Cheung, V. G. et al. (1999) Nature Genetics Supplement, 21, 15-19; Lipshutz, R. .J. et al., (1999) Nature Genetics Supplement, 21, 20-24; Bowtell, D.D.L. (1999) Nature Genetics Supplement, 21, 25-32; Singh-Gasson, S. et al. (1999) Nature Biotechnol. 17, 974-978; and, Schweitzer, B. et al. (2002) Nature Biotechnol. 20, 359-365; all of which are incorporated herein by reference. DNA chip technology is described in detail in, for instance, U.S. Pat. No. 6,045,996 to Cronin et al., U.S. Pat. No. 5,858,659 to Sapolsky et al., U.S. Pat. No. 5,843,655 to McGall et al., U.S. Pat. No. 5,837,832 to Chee et al., and U.S. Pat. No. 6,110,426 to Shalon et al., all of which are specifically incorporated herein by reference. Suitable DNA chips are available for example from Affymetrix, Inc. (Santa Clara, Calif.).

Methods for storing, querying and analyzing microarray data have for example been disclosed in, for example, U.S. Pat. No. 6,484,183 issued to Balaban, et al. Nov. 19, 2002; and U.S. Pat. No. 6,188,783 issued to Balaban, et al. Feb. 13, 2001; Holloway, A. J. et al., (2002) Nature Genetics Supplement, 32, 481-489; each of which is incorporated herein by reference.

DNA chips generally include a solid substrate or support, and an array of oligonucleotide probes immobilized on the substrate. The substrate can be, for example, silicon or glass, and can have the thickness of a glass microscope slide or a glass cover slip. Substrates that are transparent to light are useful when the method of performing an assay on the chip involves optical detection. Suitable substrates include a slide, chip, wafer, membrane, filter, sheet and bead. The substrate can be porous or have a non-porous surface. Preferably, oligonucleotides are arrayed on the substrate in addressable rows and columns. A “subarray” may thus be designed which comprises a particular grouping of probes at a particular area of the array, the probes immobilized at adjacent locations or within a defined region of the array. A hybridization assay is performed to determine whether a target DNA molecule has a sequence that is complementary to one or more of the probes immobilized on the substrate. Because hybridization between two nucleic acids is a function of their sequences, analysis of the pattern of hybridization provides information about the sequence of the target molecule. DNA chips are useful for discriminating variants that may differ in sequence by as few as one or a few nucleotides.

Hybridization assays on the DNA chip involve a hybridization step and a detection step. In the hybridization step, a hybridization mixture containing the labelled target nucleic acid sequence is brought into contact with the probes of the array and incubated at a temperature and for a time appropriate to allow hybridization between the target and any complementary probes. The array may optionally be washed with a wash mixture which does not contain the target (e.g. hybridization buffer) to remove unbound target molecules, leaving only bound target molecules. In the detection step, the probes to which the target has hybridized are identified. Since the nucleotide sequence of the probes at each feature is known, identifying the locations at which target has bound provides information about the particular sequences of these probes.

Hybridization may be carried out under various conditions depending on the circumstances and the level of stringency desired. Such factors shall depend on the specificity and degree of differentiation between target sequences for any given analysis. For example, to distinguish target sequences which differ by only one or a few nucleotides, conditions of higher stringency are generally desirable. Stringency may be controlled by factors such as the content of hybridization and wash solutions, the temperature of hybridization and wash steps, the number and duration of hybridization and wash steps, and any combinations thereof. In embodiments, the hybridization may be conducted at temperatures ranging from about 4° C. up to about 80° C., depending on the length of the probes, their G+C content and the degree of divergence to be detected. If desired, denaturing reagents such as formamide may used to decrease the hybridization temperature at which perfect matches will dissociate. Commonly used conditions involve the use of buffers containing about 30% to about 50% formamide at temperatures ranging from about 20° C. to about 50° C. An example of such a partially denaturing buffer which is commercially available is the DIG Easy Hyb™ (Roche) buffer. In embodiments, un-labelled nucleic acids such as transfer RNA (tRNA) and salmon sperm DNA may be added to the hybridization buffers to reduce background noise. Under certain conditions, a divergence of 15% over long fragments (greater than 50 bases) can be reliably detected. Single nucleotide mistmatches in shorter fragments (15 to 25 nucleotides in length) can be also detected if the hybridization conditions are designed accordingly. Hybridization time typically ranges from about one hour to overnight (16 to 18 hours approximately). After hybridization, microarrays are typically washed one to five times in buffered salt solutions such as saline-sodium citrate, abbreviated SSC, for periods of time and at salt concentrations and temperature appropriate for a particular objective. A representative procedure may for example comprise three washes in pre-warmed (50° C.) 0.1×SSC (1×SSC contains 150 mM NaCl and 15 mM trisodium citrate, pH 7). In embodiments, a detergent such as sodium dodecyl sulfate (SDS; e.g. at 0.1%) may be added to the washing buffer. Various details of hybridization conditions, some of which are described herein, are known in the art.

Hybridization may be performed under absolute or differential formats. The former refers to hybridization of nucleic acids from one sample to an array, and the detection of the nucleic acids thus hybridized. The differential hybridization format refers to the application of two samples, labelled with different labels (e.g. Cy3 and Cy5 fluorophores), to the array. In this case differences and similarities between the two samples may be assessed.

Many steps in the use of the DNA chip can be automated through use of commercially available automated fluid handling systems. For instance, the chip can be manipulated by a robotic device which has been programmed to set appropriate reaction conditions, such as temperature, add reagents to the chip, incubate the chip for an appropriate time, remove unreacted material, wash the chip substrate, add reaction substrates as appropriate and perform detection assays. If desired, the ,chip can be appropriately packaged for use in an automated chip reader.

The target polynucleotide whose sequence is to be determined is usually labelled at one or more nucleotides with a detectable label (e.g. detectable by spectroscopic, photochemical, biochemical, chemical, bioelectronic, immunochemical, electrical or optical means). The detectable label may be, for instance, a luminescent label. Useful luminescent labels include fluorescent labels, chemi-luminescent labels, bio-luminescent labels, and colorimetric labels, among others. Most preferably, the label is a fluorescent label such as a cyanine, a fluorescein, a rhodamine, a polymethine dye derivative, a phosphor, and so forth. Suitable fluorescent labels are described in for example Haugland, Richard P., 2002 (Handbook of Fluorescent Probes and Research Products, ninth edition, Molecular Probes). The label may be a light scattering label, such as a metal colloid of gold, selenium or titanium oxide. Radioactive labels such as ³²P ³³P or ³⁵S can also be used.

When the target strand is prepared in single-stranded form, the sense of the strand should be complementary to that of the probes on the chip. In an embodiment, the target is fragmented before application to the chip to reduce or eliminate the formation of secondary structures in the target. Fragmentation may be effected by mechanical, chemical or enzymatic means. The average size of target segments following fragmentation is usually larger than the size of probe on the chip.

In embodiments, the target or sample nucleic acid may be extracted from a sample or otherwise enriched prior to application to or contacting with the array. Samples may amplified by suitable methods, such as by culturing a sample in suitable media (e.g. LB) under suitable culture conditions to effect growth of microorganism(s) in the sample. Extraction may be performed using methods known in the art (see for example Sambrook et al. et al. [1989] Molecular Cloning: A Laboratory Manual.), including various treatments such as lysis (e.g. using lysozyme), heating, detergent (e.g. SDS) treatment, solvent (e.g. phenol-chloroform.) extraction, and precipitation/resuspension. In an embodiment, the nucleic acid is not amplified using polymerase chain reaction (PCR) methods prior to application to the array.

In an embodiment, the probes may be provided, for example as a suitable solution, and applied to different, discrete regions of the substrate. Such methods are sometimes referred to as “printing” or “pinning”, by virtue of the types of apparatus and methods used to apply the probe samples to the substrate. Suitable methods are described in for example U.S. Pat. No. 6,110,426 to Shalon et al. The probe samples may be prepared by a variety of methods, including but not limited to oligonucleotide synthesis, as a PCR product using specific primers, or as a fragment obtained by restriction endonuclease digestion of a nucleic acid sample. Interaction/binding of the probe to the substrate may be enforced by non-covalent interactions and covalent attachment, for example via charge-mediated interactions as well as attachment to the substrate via specific reactive groups, crosslinking and/or heating.

In an embodiment, the arrays may be produced by, for example, spatially directed oligonucleotide synthesis. Methods for spatially directed oligonucleotide synthesis include, without limitation, light-directed oligonucleotide synthesis, microlithography, application by ink jet, microchannel deposition to specific locations and sequestration with physical barriers. In general these methods involve generating active sites, usually by removing protective groups; and coupling to the active site a nucleotide which, itself, optionally has a protected active site if further nucleotide coupling is desired.

In embodiments, the probes can be bound to the substrate through a suitable linker group. Such groups may provide additional exposure to the probe. Such linkers are adapted to comprise a terminal portion capable of interacting or reacting with the substrate or groups attached thereto, and another terminal portion adapted to bind/attach to the probe molecule.

Samples of interest, e.g. samples suspected of comprising a microorganism, for analysis using the products and methods of the invention include for example environmental samples, biological samples and food. “Environmental sample” as used herein refers to any medium, material or surface of interest (e.g. water, air, soil). “Biological sample” as used herein refers to a sample obtained from an organism, including tissue, cells or fluid. Biological excretions and secretions (e.g. feces, urine, discharge) are also included within this definition. Such biological samples may be derived from a patient, such as an animal (e.g. vertebrate animal, humans, domestic animals, veterinary animals and animals typically used in research models). Biological samples may further include various biological cultures and solutions.

The probes utilized herein may in embodiments comprise a nucleotide sequence identical to a nucleic acid derived from a microorganism or substantially identical or homologous to such a nucleic acid. “Homology” and “homologous” refers to sequence similarity between two peptides or two nucleic acid molecules. Homology can be determined by comparing each position in the aligned sequences. A degree of homology between nucleic acid or between amino acid sequences is a function of the number of identical or matching nucleotides or amino acids at positions shared by the sequences. As the term is used herein, a nucleic acid sequence is “homologous” to another sequence if the two sequences are substantially identical and the functional activity of the sequences is conserved (as used herein, the term ‘homologous’ does not infer evolutionary relatedness). Two nucleic acid sequences are considered “substantially identical” if, when optimally aligned (with gaps permitted), they share at least about 50% sequence similarity or identity, or if the sequences share defined functional motifs. In alternative embodiments, sequence similarity in optimally aligned substantially identical sequences may be at least 60%, 70%, 75%, 80%, 85%, 90% or 95%. As used herein, a given percentage of homology between sequences denotes the degree of sequence identity in optimally aligned sequences. An “unrelated” or “non-homologous” sequence shares less than 40% identity, though preferably less than about 25% identity, with a sequence of interest.

Substantially complementary nucleic acids are nucleic acids in which the “complement” of one molecule is substantially identical to the other molecule. Optimal alignment of sequences for comparisons of identity may be conducted using a variety of algorithms, such as the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al., 1990, J. Mol. Biol. 215:403-10 (using the published default settings). Software for performing BLAST analysis may be available through the National Center for Biotechnology Information (through the internet at http://www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold. Initial neighbourhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program may use as defaults a word length (W) of 11, the BLOSUM62 scoring matrix (Henikoff and Henikoff, 1992, Proc. Natl. Acad. Sci. USA 89: 10915-10919) alignments (B) of 50, expectation (E) of 10 (or 1 or 0.1 or 0.01 or 0.001 or 0.0001), M=5, N=4, and a comparison of both strands. One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

An alternative indication that two nucleic acid sequences are substantially complementary is that the two sequences hybridize to each other under moderately stringent, or preferably stringent, conditions. Hybridization to filter-bound sequences under moderately stringent conditions may, for example, be performed in 0.5 M NaHPO₄, 7% sodium dodecyl sulfate (SDS), 1 mM EDTA at 65° C., and washing in 0.2 ×SSC/0.1% SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. (see Ausubel, et al. (eds), 1989, supra). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y.). Generally, stringent conditions are selected to be about 5° C. lower than the thermal melting point for the specific sequence at a defined ionic strength and pH.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”. The following examples are illustrative of various aspects of the invention, and do not limit the broad aspects of the invention as disclosed herein.

### EXAMPLES

### Example 1

### Materials and Methods

Strains and Media.

E. coli strains used to produce PCR templates are listed in Table 1. E. coli isolates including characterized strains (the non-pathogenic K12-derived E. coli strain DH5α, the enterohemorrhagic strain EDL933, the uropathogenic strain J96, the enterotoxigenic strain H-10407 and the enteropathogenic strains E2348/69 and P86-1390) and uncharacterized clinical strains from bovine (B00-4830, B99-4297), avian (Av01-4156), canine (Ca01-E179) and human (H87-5406) origin were used to assess the detection thresholds and hybridization specificity of the virulence microarray. Most of the E. coli strains were obtained from the Escherichia coli laboratory collection at the Faculté de médecine vétérinaire of the Université de Montréal. E. coli strains A22, AL851, C248 (21) were kindly provided by Carl Marrs (University of Michigan) and IA2 by J. R. Johnson (University of Minnesota) respectively. All strains were stored in Luria-Bertani broth (LB [6]) broth plus 25% glycerol at −80° C. E. coli cultures were grown at 37° C. in LB broth for genomic DNA extraction and purification.

Selection and Sequence Analysis of Virulence Gene Probes.

The selection included virulence genes of E. coli pathotypes involved in intestinal and extra-intestinal diseases in humans and animals (see Table 1). The primers used for probe amplification were either chosen from previous studies on virulence gene detection or designed from available gene sequences (see Table 2). 103 E. coli virulence genes were targeted in this study, encoding (a) toxins (heat-labile toxin LT, human heat-stable toxin STaH, porcine heat-stable toxin STaP, Shiga-toxins Stx1 and Stx2, haemolysins Hly and Ehx, East1, STb, EspA, EspB, EspC, cytolethal distending toxin Cdt, cytotoxic necrosing factor Cnf, Cva, Leo) (b) adhesion factors (Cfa, Iha, Pap, Sfa, Tir, Bfp, Eaf, Eae, Agg, Lng, Aida, Foc, Afa, Nfa, Drb, Fim, Bma, ClpG, F4, F5, F6, F17, F18, F41) (c) secretion systems (Etp) (d) capsule antigens (KfiB, KpsMTII, KpsMTIII, Neu) (e) somatic antigens (RfcO4, RfbO9, RfbO101, RfbO111, RfbEO157) (f) flagellar antigen (FliC), (g) invasins (IbeA, IpaC, InvX), (h) autotransporters (Tsh), (i) aerobactin system (IucD, TraT, IutA) and, in addition, to espP (serine-protease), katP (catalase), omp (outer membrane proteins A and T), iroN (catechol siderophore receptor), iss (serum survival gene), putative RTX family exoprotein (rtx) and paa (related attaching and effacing gene) probes. The Yersinia high-pathogenicity island (irp1, irp2, and fyuA) present in different E. coli pathotypes and other Enterobacteriaceae was also targeted (3). An E. coli positive control gene, uidA, which encodes the E. coli-specific β-glucuronidase protein (17, 31) and the uspA gene which encodes a uropathogenic-specific protein (60) were added to this collection.

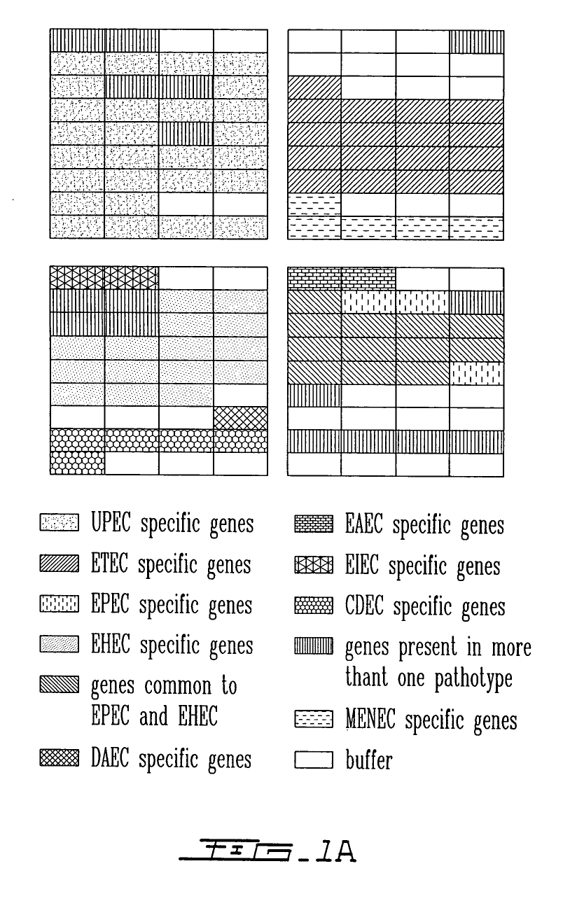

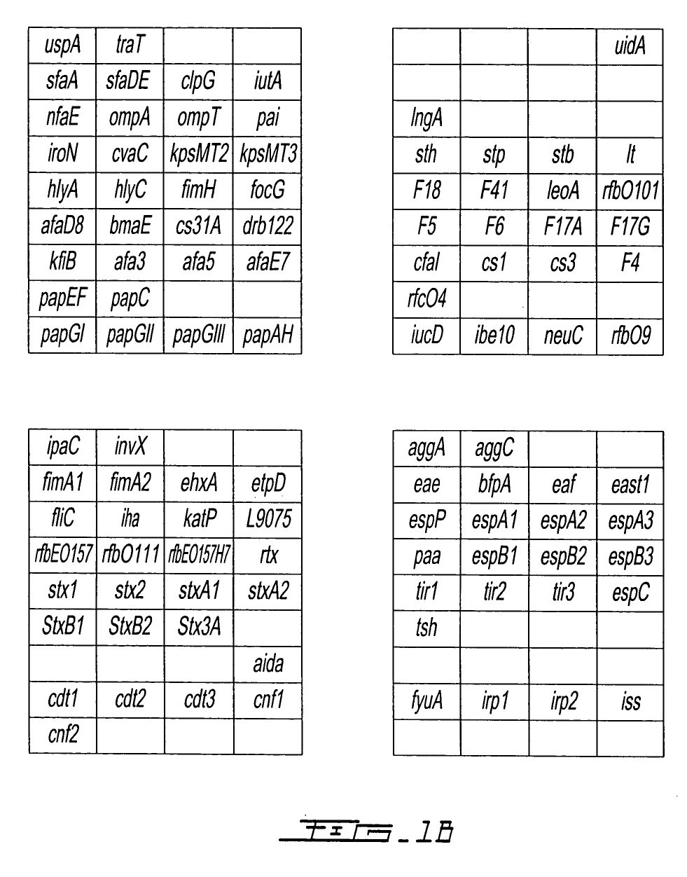

The DNA sequence of each gene was analyzed by BLAST analysis and ClustalW alignment followed by phylogenetic analysis. When the selected gene showed sequence divergence over 10% amongst different strains, new primers were designed to amplify the probe from each phylogenetic group as was the case for espA, espB and tir genes. The new primers were selected in conserved sequence areas flanking the area of divergence in order to ensure gene discrimination at the hybridization level. Phylogenetic analysis of the attaching and effacing locus (LEE) genes espA, espB and tir permitted us to distinguish three phylogenetic groups with regard to the sequence divergence cutoff value (<10%) ,chosen for this study. Attaching and effacing genes from strains EDL933, E2348/69 and RDEC-1 belonging to the different phylogenetic groups have been cloned and sequenced (27, 76, 92). Genomic DNA from strains EDL933 (EHEC), E2348/69 (Human EPEC) and RDEC-1 (rabbit EPEC) were used as templates to PCR amplify the different probes espA2-espB1-tir2, espA3-espB2-tir3 and espA1-espB3-tir1 respectively. The amplified probes were sequenced to confirm their identity and printed onto the pathotype microarray as shown in FIG. 1. For some virulence determinants, several genes of the cluster were targeted such as hly (hlyA, hlyC), pap (papAH, papEF, papC, papG), sfa (sfaDE, sfaA), agg (aggA, aggC). Utilization of several genes per cluster assisted in the confirmation of positive signals in addition to the assessment of cluster integrity. DNA probes detecting the genetic variants of Shiga-toxins (stx1, stx2, stxA1, stxA2, stxB1 and stxB2), cytolethal distending toxin (cdt1, cdt2 and cdt3), cytotoxic necrosing factor (cnf1, cnf2), and papG alleles (papGI, papGII and papGIII) were also included. In total, this gene sequence analysis resulted in the selection of 105 gene probes (Table 1). Probe amplification, purification and sequencing.

E. coli strains were grown overnight at 37° C. in Luria-Bertani medium. A 200 μl sample of the culture was centrifuged, the pellet was washed and resuspended in 200 μl of distilled water. The suspension was boiled 10 min and centrifuged. A 5 μl aliquot of the supernatant was used as a template for PCR amplification. PCR reactions were carried out in a total volume of 100 μl containing 50 pmol of each primer, 25 μmol of dNTP, 5 μl of template, 10 μl of 10× Taq buffer (500 mM KCl, 15 mM MgCl₂, 100 mM Tris-HCl, pH 9) and 2.5 U of Taq polymerase (Amersham-Pharmacia). PCR products were analyzed by electrophoresis on 1% agarose gels in TAE (40 mM Tris-acetate, 2 mM Na₂EDTA), then purified with the Qiaquick™ PCR Purification Kit (Qiagen, Mississauga, Ontario) and eluted in distilled water. Since the annealing temperature of the various PCR primers ranged from 40° to 65° C. and genomic DNA from 36 E. coli strains were used as template, all the PCR amplifications were done separately. A total of 103 virulence factor probes and two positive control probes, uidA and uspA, were amplified successfully as determined by amplicon size and DNA sequence. The purity of the amplified DNA was confirmed by agarose gel electrophoresis of 50-100 ng of each amplified fragment. The size of the PCP products ranged from 117 bp (east1) to 2121 bp (katP) with an average length of 500 bp for the majority of the DNA probes (Table 1). For quality control purposes all PCR fragments were partially sequenced for gene verification (Applied Biosystem 377 DNA sequencer using the dRhodamine Terminator Cycle Sequencing Ready™ reaction Kit).

Genomic DNA Extraction and Labeling.

Cells, collected by centrifuging 5 ml of an overnight culture, were washed with 4 ml of solution 1 (0.5 M NaCl, 0.01 M EDTA pH 8), resuspended in 1.2 ml of buffer 2 (solution 1 containing 1 mg/ml of lysozyme), then incubated at room temperature for 30 min. After SDS addition and phenol-chloroform extraction, total DNA was precipitated by adding one volume of isopropanol. The harvested pellet was washed with one volume of 70% ethanol, dried then resuspended in 100 μl of Tris-EDTA buffer. Before labeling, tot al DNA was reduced in size by restriction enzyme digestion (New England BioLabs, Mississauga, Ontario) and following digestion, the enzymes removed by phenol-chloroform extraction. Cy 3 dye was covalently attached to DNA using a commercial chemical labeling method (Mirus' Label IT™, PANVERA) with the extent of labeling depending primarily on the ratio of reagent to DNA and the reaction time. These parameters were varied to generate labeled DNA of different intensity. Two μg of the digested DNA were chemically labeled using 4 μl of Label IT™ reagent, 3 μl of 10× Mirus™ labeling buffer A and distilled water in a 30 μl total volume. The reactions were carried out at 37° C. for 3 h. Labeled DNA was then separated from free dye by washing four times with water and centrifugation through Microcon™ YM-30 filters (Millipore, Bedford, USA). The amount of incorporated fluorescent cyanine dye was quantified by scanning the probe from 200 nm to 700 nm and subsequently inputting the data into the % incorporation calculator found at http://www.pangloss.com/seidel/Protocols/percent inc.html. This method is based on the calculation of the ratio of μg of incorporated fluorescence: μg of labeled DNA.

Optimization of Microarray Detection Threshold Using a Prototype Microarray

A prototype chip was constructed and used to assess parameters, namely fragment length and extent of fluorescent labeling of the target (test) DNA, to optimize the spot detection threshold of the microarray. DNA amplicons from 34 E. coli virulence genes including the following EHEC virulence gene probes: espP (13), EHEC-hlyA (80), stx1 (84), stx2 (91), stxc (91), stxall (46), paa (1) and eae (4), were generated by PCR amplification and printed in triplicate. The probe lengths ranged from 125 bp (east1) to 1280 bp (irp1). A HindIII/EcoRI digestion was used to generate large fragments (average size ˜6 Kb) and Sau3A/AluI digestion to produce smaller DNA fragments (average size ˜0.2 Kb) from E. coli O157:H7 strain STJ348 genomic DNA. The restricted DNAs were labeled and used as the target for hybridization with the prototype microarray. In our experiments, the strongest hybridization signal was obtained by using larger fragments labeled at an optimal Cy3 rate in the range of 7.5 to 12.5. An estimate of the microarray's sensitivity was calculated by the following equation as described by De Boer and Beumer (24):

Sensitivity (%)=(number of true positive spots (p)/p+number of false negative spots)×100

Construction of the E. coli Pathotype Microarray

Virulence factor probes were grouped by pathotype with the resulting array being composed of eight subarrays each corresponding to well characterized E. coli categories (FIG. 1). The enterohemorrhagic (EHEC) subarray included Shiga-toxin gene probes (stx1, stx2, stxA1, stxA2, stxB1, stxB2 and stxB3), attaching and effacing genes, (espA, espB, tir, eae, and paa), EHEC specific pO157 plasmid genes (etpD, ehxA, L9075, katP, espP) and O157 and O111 somatic antigen genes (rEbEO157 and rfbO111). Enteropathogenic E. coli (EPEC) was targeted by spotting LEE specific gene probes (eae, tir, espA, espB), espC and EPEC EAF plasmid probes (bfpA, eaf). The enterotoxigenic subarray (ETEC) included probes for human heat-stable toxin (STaH), porcine heat-stable toxin (STaP), heat-stable toxin type II (STb), heat-labile toxin (LT), adhesion factors shared by human ETEC (CFAI, CS1, CS3, LngA) or by animal ETEC (F4, F5, F6, F18, F41). DNA probes for O101 specific somatic antigen (rfbO101) and ETEC toxin (leoA) were also included. To identify uropathogenic strains, the UPEC subarray was composed of 27 probes selected for detection of extraintestinal E. coli adhesins Pap (papGI, papGII, papGIII, papAH, papEF, papC), Sfa (sfaA, sfaDE), Drb (drb122), Afa (afa3, afa5, afaE7, afaD8), F1C (focG), nonfimbrial adhesin-1 (nfaE), M-agglutinin subunit (bmaE), CS31A (clpG), toxins including hemolysins (hlyA and hlyC), cytotoxic necrosing factor (cnf1), and colicin V (cvaC), aerobactin receptor (iutA), capsular specific genes kfiB (K5), kpsMTII (K1, K5, K12), KpsMTIII (K10, K54) in addition to the surface exclusion gene (traT) and uspA probes. The cell-detaching subarray (CDEC) contained toxin probes cnf1, cnf2, cdt1, cdt2 and cdt3. The genes iucD, neuC, ibe10, rfbO9 and rfcO4 were designed to represent the meningitis-associated E. coli pathotype (MENEC). Enteroaggregative E. coli probes (EAEC) were derived from fimbrial specific genes aggA and aggC whereas enteroinvasive pathotype (EIEC) was targeted by invasin gene probes ipaC and invX. The AIDA (adhesin involved in diffuse adherence) probe was the unique marker for the diffusely adherent pathotype (DAEC).

Some virulence genes, such as fimA, fimH, irp1, irp2, iss, fyuA, ompA, east1, iha, fliC, tsh and ompT are shared by several E. coli pathotypes, and are thus indicative of subsets of pathotypes rather than specific to any one pathotype in particular. Finally a positive control, the uidA gene probe (17, 31) as well as a negative control composed of 50% DMSO solution were added. An estimate of the specificity of the virulence microarray was calculated by the following equation (24):

Specificity (%)=(number of true negative spots (n)/n+number of false positive spots)×100

Printing and Processing of the Microarrays.

Two μg of each DNA amplicon were lyophilized in a speed-vacuum and resuspended in filtered (0.22 μm) 50% DMSO. The concentration of amplified products was adjusted to 200 ng/μl and 10 μl of each DNA amplicon was transferred to a 384-well microplate and stored at −20° C. until the printing step. DNA was then spotted onto CMT-GAPS™ slides (Corning Co., Corning, N.Y.) using a VIRTEK ChipWriter™ with Telechem SMP3™ microspotting pins. Each DNA probe was printed in triplicate on the microarray. After printing, the arrays were subjected to ultraviolet crosslinking at 1200 μJoules (U.V. Stratalinker™ 1800, STRATAGEN) followed by heating at 80° C. for four hours. Slides were then stored in the dark at room temperature until use.

Microarray Hybridization and Analysis.

Microarrays were prehybridized at 42° C. for one hour under a 22×22 mm coverslip (SIGMA) in 20 μl of pre-warmed solution A (DIG Easy Hyb™ buffer, Roche, containing 10 μg of tRNA and 10 μg of denatured salmon sperm DNA). After the coverslip was removed by dipping the slide in 0.1×SSC (1×SSC contained 150 mM NaCl and 15 mM trisodium citrate, pH 7), the array was rinsed briefly in water and dried by centrifugation at room temperature in 50 ml conical tubes for five min at 800 rpm. Fluorescently-labeled DNA was chemically denatured as described by the manufacturer and added to 20 μl of a fresh solution of pre-warmed solution A. Hybridization was carried out overnight at 42° C. as recommended by the manufacturer. After hybridization, the coverslip was then removed in 0.1×SSC and the microarray washed three times in pre-warmed 0.1×SSC/0.1% SDS solution and once in 0.1×SSC for 10 min at 50° C. After drying by centrifugation (800 rpm, five min, room temperature), the array was analyzed using a fluorescent scanner (Canberra-Packard, Mississauga, Ontario). The slides were scanned at a resolution of 5 μm at 85% laser power and the fluorescence quantified after background subtraction using QuantArray™ software (Canberra-Packard). All hybridization experiments were replicated between two to five times per genome.

### Example 2

### Assessment of the Pathotype Microarray for Virulence Pattern Analysis

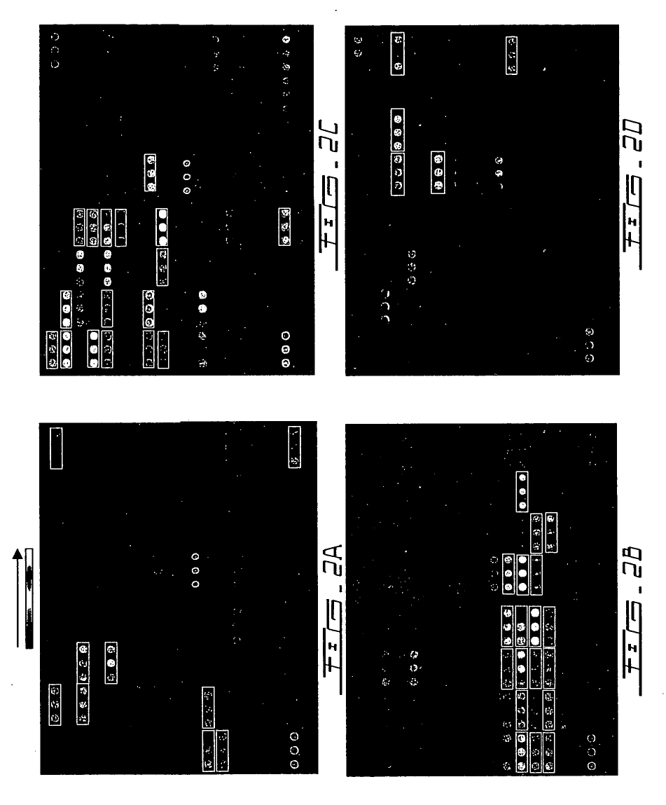

To identify known virulence genes and consequently, the pathotype of the E. coli strain being examined, genomic DNA from several previously characterized E. coli strains was labeled and hybridized to the pathotype microarray. The K12-derived E. coli strain DH5α was included as a nonpathogenic control. Interestingly, E. coli DH5α produced a fluorescent hybridization signal with the uidA, fimA₁, fimA₂, fimH, ompA, ompT, traT, fliC and iss probes (FIG. 2A). Genbank analysis of the sequenced K12 strain MG1655 genome revealed the presence of the first seven genes whereas the iss probe is 90% similar to ybcU, a gene encoding a bacteriophage lambda Bor protein homolog (sequence K12) . Surprisingly, a false positive signal was obtained with the cdt1 and aggA gene probes. These genes are absent in the E. coli K12 genome and their sequences are not homologous to any K12 genes. Moreover, these genes were not positive with K12 or O157:H7 strain EDL933 in earlier generations of the virulence chip. We postulated that the signal may have been the result of amplicon contamination in the final printing. Therefore, these two probes were not included in all subsequent hybridization analyses.

Since the genomic sequence of E. coli O157:H7 strain EDL933 is available on GENBANK (NC_{—}002655), this strain represented a good choice to assess the detection threshold and hybridization specificity of the E. coli virulence factors on the microarray. After hybridizing the pathotype microarray with Cy3-labeled genomic DNA from E. coli O157:H7, the scanned image (FIG. 2B) showed fluorescent signals with the EHEC specific genes encoding Shiga-toxins, the attaching and effacing cluster present in EHEC and EPEC E. coli, the genes carried on the EHEC pO157 plasmid, antigen and flagellar specific genes as well as iha, an adhesin encoding gene (AF401752) found in both the EHEC and UPEC pathotypes. Therefore the EHEC pathotype of E. coli 0157:H7 was easily confirmed by a rapid visual scan of the virulence gene pattern (FIG. 1) of the scanned image.

The UPEC strain J96 (04:K6) is a prototype E. coli strain from which various extraintestinal E. coli virulence factors have been cloned and characterized (73, 86). This strain possesses two copies of the gene clusters encoding P (pap-encoded) and P-related (prs-encoded) fimbriae, produces F1C (focG), contains two hly gene clusters encoding hemolysin and produces cytotoxic necrosing factor type 1 (cnf1). E. coli strain J96 DNA was labeled and hybridized to the pathotype microarray. The scanned array resulted in a UPEC pathotype hybridization pattern (FIG. 2C) . All of the UPEC virulence genes cited above were detected, as well as other uropathogenic specific genes. From a taxonomic perspective, the microarray also permitted the detection of the 04 antigen gene (rfcO4).

An enterotoxin-producing strain of E. coli isolated from a case of cholera-like diarrhea, E. coli strain H-10407 (30), was used as a control strain to assess the ability of the microarray to identify the ETEC pathotype (FIG. 2D). Hybridization results showed the presence of a heat-stable enterotoxin Stah, antigenic surface-associated colonization factor cfaI, heat-labile enterotoxin LT, east1 toxin, and a weak signal was obtained with stap probe. The hybridization pattern correlated well with the virulence profile and pathotype group of this strain (28, 29, 68).

### Example 3

### Determination of Virulence Patterns of Uncharacterized Clinical E. coli Strains

To further validate the pathotype chip, virulence gene detection was assessed by hybridization with genomic DNA from five clinical E. coli strains isolated from human (H87-5406) and animal (Av01-4156, B00-4830, Ca01-E179, B99-4297) sources. Genomic DNAs from these strains were fragmented and Cy3-labeled and the microarray hybridization patterns obtained were compared with PCR amplification results.

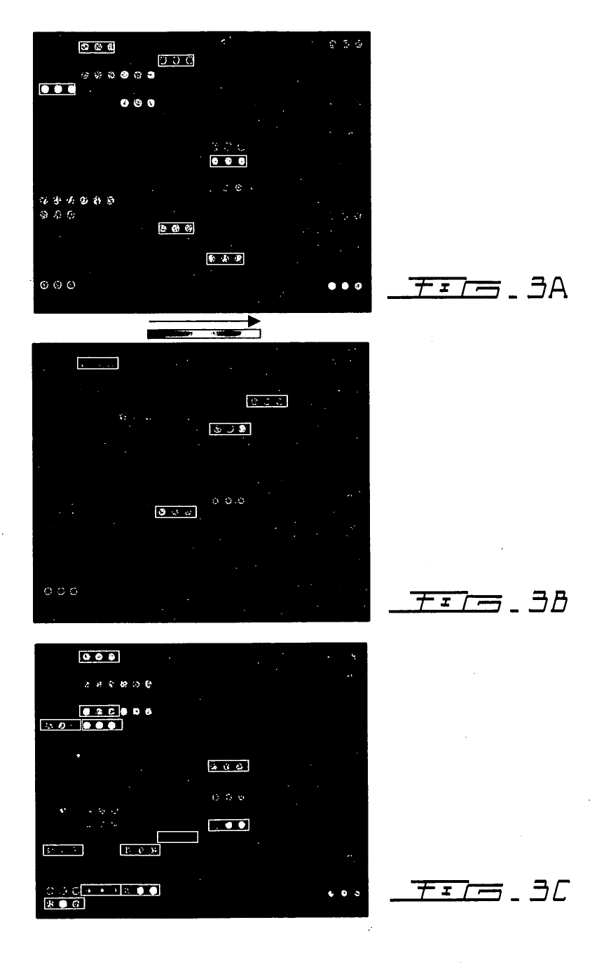

The virulence gene pattern obtained after microarray hybridization analysis with Cy3-labeled E. coli genomic DNA of avian-origin (Av01-4156) showed the presence of the extra-intestinal E. coli virulence genes (iucD, iroN, traT, iutA) and genes present in our K12 strain (fimA₁, fimA₂, fimH, iss, ompA, and ompT) (FIG. 3A). The temperature-sensitive hemagglutinin gene (tsh) that was often located on the ColV virulence plasmid in avian-pathogenic E. coli (APEC) (26) was also detected on the Av01-4156 virulence gene array. A strong hybridization signal was also obtained with the rtx probe derived from a gene located on the O157:H7 chromosome and encoding a putative RTX family exoprotein. The overall virulence factor detection pattern indicates that this strain is involved in extraintestinal infections.

When the pathotype microarray was hybridized with genomic DNA from strain B00-4830 isolated from bovine ileum, genes encoding ETEC fimbriae F5 and heat stable toxin StaP were detected (FIG. 3B) indicating that this strain belongs to animal ETEC pathotype. The hybridization pattern also showed the presence of traT, ompA, fimA₁, fimA₂, fimH, fliC genes and the EHEC-associated gene etpD.

The virulence pattern obtained after microarray hybridization analysis with Cy3-labeled human-origin E. coli genomic DNA H87-5406 strain was very complex and did not fall within a single pathotype category. The hybridization pattern revealed the presence of espP, iss, rtx, fimA₁, fimA₂, fimH, ompA, and ompT genes as well as Shiga-toxin gene, stx1, detected in the enterohemorragic pathotype (FIG. 3C). Moreover, virulence genes involved in extra-intestinal infections (cdt2, cdt3, afaD8, bmaE, iucD, iroN, traT and iutA) were also observed. Strain H87-5406 was also positive for the type 2 cytotoxic necrosing factor encoded by cnf2 gene.

The virulence patterns of two other isolates, the pulmonary isolated strain Ca01-E179 and the bovine strain B99-4297 (used elsewhere in this study) were clearly identified as UPEC pathotype and Shiga-toxin positive E. coli respectively (data not shown). The presence of all the pathotype-specific virulence factors that were positively identified by the microarray data for the above animal and human isolates, was further confirmed by PCR amplification of each positive signal.

### Example 4

### Discrimination Between Homologous Genes Belonging to Different Subclasses

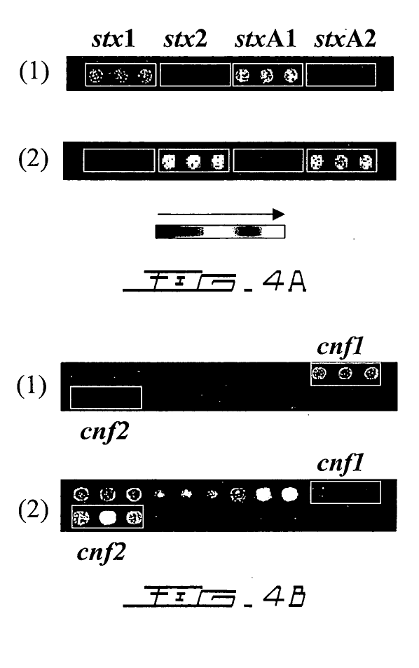

Given the importance of the stx gene family, amplicons stxA1 and stxA2 specific for the A subunits of the stx1 and stx2 family (Table 2) were designed, in addition to using the published amplicons stx1 and stx2 (Table 1) which overlap the A and B subunits of the genes. Sequence similarity is of the order of 57% between the published stx1 and stx2 amplicons; similarity between the stxA1 and stxA2 amplicons designed herein is slightly higher, at 61%. As shown in FIG. 4A, the DNA probes used in this study for detection of stx1 and stx2 gene variants were successful in distinguishing stx1 from stx2, using either the previously published amplicons or the stxA subunit probes.

To further explore the potential of microarrays to distinguish gene variants within homologous gene families, primers used for cnf1and cnf2 probe amplification were derived from studies on the detection of cnf variant genes by PCR amplification. The resulting amplicons have 85% sequence similarity. Hybridization results obtained with genomic DNA from cnf-positive strains H87-5406 and Ca01-E1799 (FIG. 4B) showed a clear distinction on the microarray between cnf1and cnf2 gene variants, a significant result given the high degree of similarity and the size (over 1 kb) of the amplicons used.

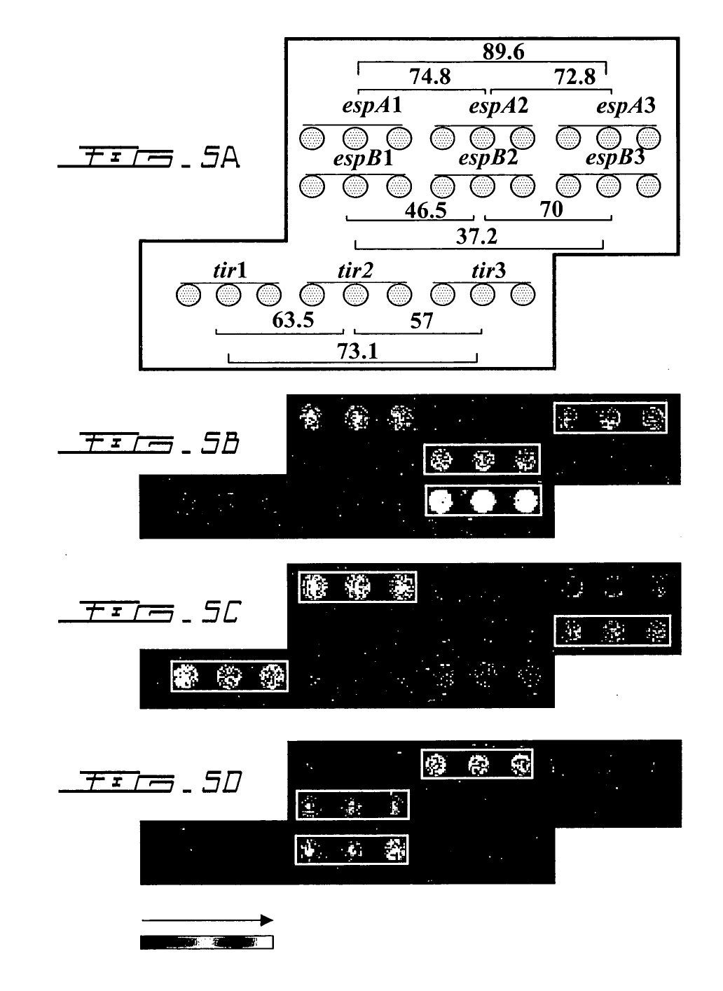

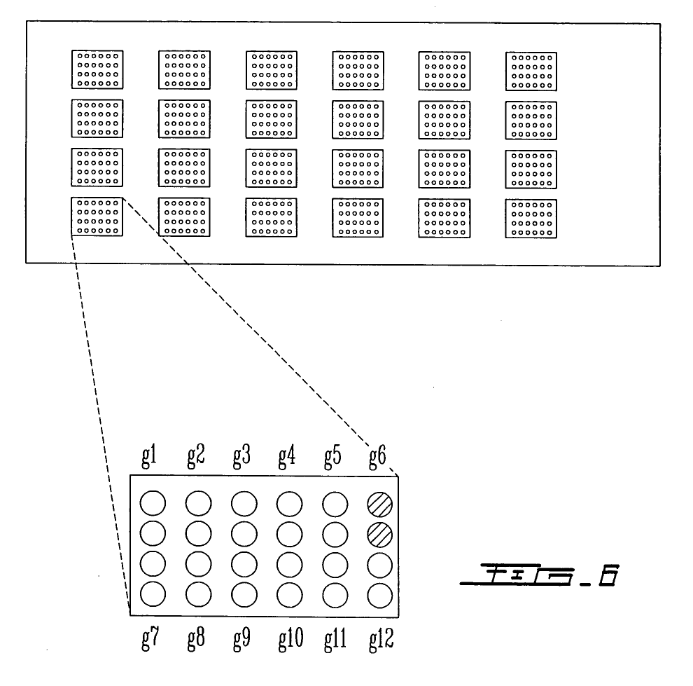

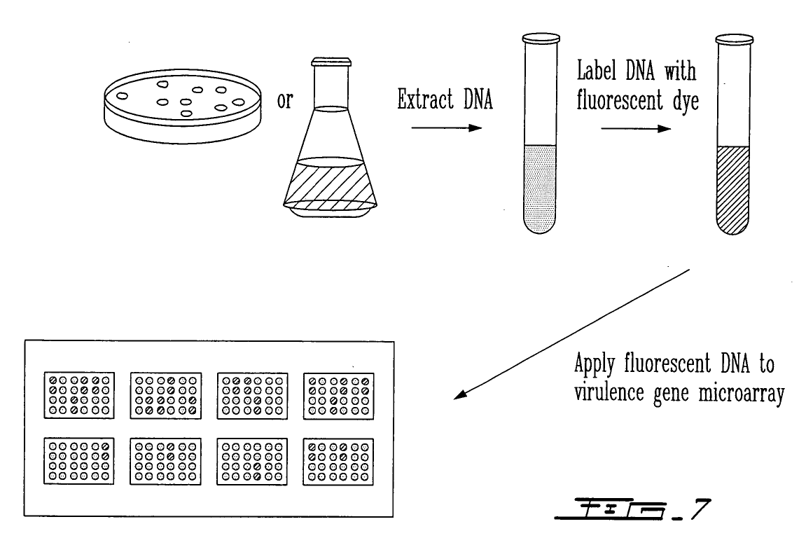

Since the DNA microarray showed initial promise in discriminating between the known gene variants of stx and cnf, a more defined group of genes were selected in order to test the ability of the pathotype microarray to differentiate between different phylogenetic groups of genes with a sequence divergence cutoff value of >10%. The DNA sequence similarity values of espA, espB and tir probes from the three different groups are summarized in FIG. 5A. The microarray was hybridized with labeled genomic DNA from EDL933 (EHEC) and E2348/69 (EPEC1) strains. Labeled DNA from another strain P86-1390 belonging to the same phylogenetic group as RDEC-1 was used to validate the hybridization specificity of the arrayed virulence genes. Hybridizations with the pathotype microarray were performed at 42° C. and 50° C. and, as shown in FIG. 5B, C and D, the labeled DNA hybridized as expected to probes specific for each phylogenetic group. Genomic DNA from strain P86-1390 hybridized with espA1-espB3-tir1 probes, indicating that this strain belongs to the same group as RDEC-1, which correlates well with the phylogenetic analysis. A strong cross-hybridization signal was obtained between the espA1 and espA3 probes due to their high DNA-similarity score (89.6%). These hybridization patterns were obtained at 42° C. as well as at 50° C. suggesting that DNA sequence divergences of 25% can be resolved under standard hybridization conditions. These results demonstrated that the pathotype microarray can be a useful tool for strain genotyping.

The studies described herein entailed designing a DNA microarray containing 102 gene probes distributed into eight subarrays corresponding to various E. coli pathotypes. To evaluate the microarray regarding the specificity of the amplified virulence factor gene fragments, genomic DNAs from different E. coli strains were labeled and hybridized to the virulence factor microarray. To this end, applicants developed a simple protocol for probe and target preparation, labeling and hybridization. The use of PCR amplification for probe generation, and fragmented genomic DNA as labeled target allowed the detection of all known virulence factors within characterized E. coli strains. Direct chemical labeling of genomic DNA with a single fluorescent dye (Cy3) facilitated the work.

Since the fluorescent assay used herein was based on direct detection (single Cy dye) rather than differential hybridization (multiple dyes), optimization of the signal detection threshold was performed. It was determined that the signal intensity, apart from DNA homology and DNA labeling efficiency, depended on (i) immobilized amplicon size (ii) gene copy number in target genomic DNA and (iii) size of the labeled target DNA. Within the large range of probe sizes (117 bp and 2121 bp) tested, hybridization signal intensity could be affected by probe length when using homologous DNA. Quality control analysis of the printed microarray using terminal transferase showed heterogeneity in the spotted amplicons. Since this enzyme attaches Cy3 to the 3′ end of the fixed DNA amplicon, we expected that the quality control signal would be stronger with smaller amplicons due to an increased number of free ends. Unexpectedly however, small fragments (less than 200 bp) produced poorer hybridization signals than that of larger amplicons. Using two strains with known genomes (K12 and EDL933), we can estimate the level of accuracy (sensitivity and specificity) of the current virulence chip as outlined in the Examples herein. The average sensitivity or accuracy in discriminating among the different virulence genes approached 97%. These estimates take into account a shared total of three false negatives among the total of 210 (i.e. 2×105) virulence gene spots for both strains.

Gene location is another factor to consider when designing gene detection microarrays. After hybridization with genomic DNA from E. coli O157:H7 strain EDL933, it was found strong hybridization signals to etpD, ehxA, L7590, katP and espP. Since these genes are located on the pO157 plasmid (Accession number AF074613) (15) the stronger signal can be attributed to a higher copy number or gene dose. Moreover, many virulence genes are located on mobile elements like plasmids, phages, or transposons (69) and are encoded by foreign DNA acquired via horizontal gene transfer and inserted in the genome. These pathogenicity islands (PAIs) are highly unstable and are constantly shuttled between strains. However, in addition to their total horizontal transfer (12, 38) or deletion (10, 11, 40), several studies suggested that PAIs are subject to continuous modifications in their virulence factor composition (52). In earlier work, the detection of a single. PAI gene reflected the presumed presence of all the additional virulence genes encoded by the PAI (59) but due to the potential for genetic rearrangements described above, this assumption is risky. Microarray technology represents an excellent tool to circumvent this PAI plasticity and identify genetic rearrangements by gene deletion or insertion on PAI clusters.

Recent investigations of E. coli virulence have revealed new information regarding the prevalence of virulence genes within a specific E. coli pathotype. For example the cytolethal-distending factor (cdt) was first described as virulence factor associated with EPEC E. coli and other diarrhea-associated pathotypes (2, 56, 57) . Later, this gene was detected in strains involved in extraintestinal infections in humans and dogs (49-51, 54, 55). More recently, cdt and the urinary tract infection-associated gene (ompT) have been found to be as or more prevalent than traditional neonatal bacterial meningitis NBM-associated traits, such as ibeA, sfaS, and K1 capsule (52). The usefulness of the virulence microarray concept for exploring the global virulence pattern of strains and the potential detection of unexpected virulence genes was revealed by total genomic hybridizations with uncharacterized clinical strains. The rtx probe (encoding a putative RTX family exoprotein, accession number AE005229) located on the O157:H7 chromosome was amplified using genomic DNA from strain EDL933. Blast analysis did not reveal significant similarities with any available sequences. Analysis of the hybridization patterns of the extraintestinal strain Av01-4156 and strain H87-5406 revealed a strong signal with the rtx probe indicating the presence of a gene homologous to the rtx probe (FIG. 3). This gene was successfully amplified in both strains using the rtx-specific primers. To our knowledge, this is the first report of the presence of this gene in non-O157 strains.

The potential for possessing different combinations or sets of virulence genes within a given E. coli strain could lead to the emergence of new pathotypes. Consistent with this hypothesis, it was found that in the clinical strain H87-5406, a combination of virulence factors from different pathotypes was observed. Moreover, microarray hybridization permitted detection of the Shiga-toxin gene stx1 associated with EHEC strains in addition to virulence genes involved in extra-intestinal infections (cdt2, cdt3, afaD8, bmaE, iucD, iroN, traT, iutA). Starcic et al. (83) recently reported a case of a “bifunctional ” E. coli strain isolated from dogs with diarrhea. When analyzed, only a few strains were positive for heat stable toxin (ST) and none of them produced diarrhea-associated fimbriae K88 or K99 in contrast with previous studies (85). However, most of these strains were positive for cytonecrosing toxin (cnf1) as well as P-fimbriae and hemolysin (hly) that are involved in extra-intestinal infections in humans and animals. It was thus concluded that hemolytic E. coli isolated from dogs with diarrhea have characteristics of both uropathogenic and necrotoxigenic strains.

Another example illustrating the ability of the virulence microarray to provide a more thorough analysis of virulence genes and consequently the detection of potentially new pathotypes is further supported by the present study in which the ETEC pathotype of the bovine clinical strain B00-4830 was confirmed. In addition to the presence of the ETEC-associated virulence genes encoding StaP and F5 revealed in the hybridization pattern, the etpD gene, described by Schmidt et al. (82) as an EHEC type II secretion pathway, was unexpectedly found to be present. In their study, Schmidt et al. (82), reported that the etp gene cluster was detected in all 30 of the EHEC strains tested by hybridization (using the 11.9 Kb etp cluster from EDL933 as a probe) and by PCR using etpD-specific primers. However, none of the other E. coli pathotypes tested (EPEC, EAEC, EIEC, and ETEC) were positive for the etp gene cluster. As our results are contrary to this study, we assayed for the presence of the etpD gene in strain B00-4830 by PCR using the reverse primer described by Schmidt et al (82) and a forward one designed in our study. Amplification of the expected 509 bp fragment was consistent with the microarray results confirming that that etpD gene can be found in ETEC strains.

Another unexpected finding of the study described herein was the prevalence of fimH and ompT genes that have been epidemiologically associated with extraintestinal infections (51, 54). BLAST analysis of ompT and fimH genes indicated the presence of both genes in E. coli K12 strain MG1655 and in enterohemorrhagic E. coli O157:H7 strain EDL933 and strain RIMD 0509952. In addition, the hybridization results herein revealed the presence of the fimH gene in all strains tested in this study, including non-pathogenic E. coli, EPEC, ETEC and UPEC strains. The ompT gene was less prevalent but present in the Shiga-toxin producing strain H87-5406. It was also found in another Shiga-toxin producing strain B99-4297 as well as in the EPEC strains P86-1390 and E2348/69. The use of these genes as indicators of the UPEC pathotype should be reconsidered.

The studies described herein thus demonstrate that DNA microarray technology can be a valuable tool for pathotype identification and assessing the virulence potential of E. coli strains including the emergence of new pathotypes. The DNA chip design described herein should facilitate epidemiological and phylogenetic studies since the prevalence of each virulence gene can be determined for different pathotypes (and strains) and the phylogenetic associations elucidated between virulence pattern and serotypes of a given strain. In addition, unlike traditional hybridization formats, microchip technology is compatible with the increasing number of newly recognized virulence genes since thousands of individual probes can be immobilized on one glass slide.

The DNA labeling methodology, hybridization and pathotype assessment described herein is both rapid and sensitive. The applications of such microarrays extend broadly from the medical field to drinking water, food quality control and environmental research, and can easily be expanded to virulence gene detection in a variety of pathogenic microorganisms.

TABLE 1
Genes targeted, primers sources and strains used as
PCR amplification templates in this study.

| | | | SEQ | | Reference |
|---|---|---|---|---|---|
| | Accession | Size | ID | | of |
| Gene | number | (bp) | NO: | Strains | primers |
| afaBC3 | X76688 | 793 | 1 | A22 | (22, 62) |
| afaE5 | X91748 | 470 | 2 | AL 851 | This |
| | | | | | study |
| afaE7 | AF072901 | 618 | 3 | 262-KH 89 | (61) |
| afad8 | AF072900 | 351 | 4 | 2787 | (61) |
| agga | U12894 | 432 | 5 | Strain 17.2 | (78) |
| aggc | U12894 | 528 | 6 | Strain 17.2 | (78) |
| aida | X65022 | 644 | 7 | 2787 | (5) |
| bfpa | U27184 | 324 | 8 | O126: H6 E2348/69 | (39) |
| bmae | M15677 | 505 | 9 | 215 | (54) |
| cdt1 | U03293 | 412 | 10 | O15: KRVC383 | This |
| | | | | OvinS5 | study |
| cdt2 | U042208 | 556 | 11 | O15: KRVC383 | This |
| | | | | OvinS5 | study |
| cdt3 | U89305 | 556 | 12 | O15: KRVC383 | This |
| | | | | OvinS5 | study |
| cfai | S73191 | 479 | 13 | H-10407 cfaI | This |
| | | | | | study |
| clpg | M55389 | 403 | 14 | 215 | (7) |
| cnf1 | X70670 | 1112 | 15 | J96 O4: K12 | (74) |
| cnf2 | U01097 | 1240 | 16 | O15: KRVC383 | (74) |
| | | | | OvinS5 | |
| cs1 | M58550 | 321 | 17 | PB-176P cfa−II | This |
| | | | | | study |
| cs3 | M35657 | 401 | 18 | PB-176 cfa+ II | This |
| | | | | | study |
| cs31a | M59905 | 710 | 19 | 31a | (37) |
| CvaC | X57525 | 680 | 20 | 1195 | (54) |
| derb122 | U87541 | 260 | 21 | O4: K12 J96 | This |
| | | | | | study |
| eae | U66102 | 791 | 22 | O157: H7 STJ348 | (4) |
| eaf | X76137 | 397 | 23 | O126: H6 E2348/69 | (32) |
| east1 | L11241 | 117 | 24 | O149: K9 1P97- | (90) |
| | | | | 2554B | |
| ehxa | AF043471 | 158 | 25 | O157: H7 STJ348 | (31) |
| espa | AF064683 | 478 | 26 | P86-1390 | This |
| group I | | | | | study |
| espA | AF071034 | 523 | 27 | O157: H7 EDL933 | This |
| group | | | | | study |
| II | | | | | |
| espA | AJ225016 | 481 | 28 | O126: H6 E2348/69 | This |
| group | | | | | study |
| III | | | | | |
| espB | AF071034 | 502 | 29 | O157: H7 EDL933 | This |
| group I | | | | | study |
| espB | Z21555 | 377 | 30 | O126 H6 E2348/69 | This |
| group | | | | | study |
| II | | | | | |
| espB | X99670 | 395 | 31 | P86-1390 | This |
| group | | | | | study |
| III | | | | | |
| espC | AF297061 | 500 | 32 | O126 H6 | This |
| | | | | E2348/69 | study |
| espP | AF074613 | 1830 | 33 | 215 | (13) |
| etpD | Y09824 | 509 | 34 | O157: H7 EDL933 | (82), |
| | | | | | this |
| | | | | | study |
| F17A | AF022140 | 441 | 35 | O15: KRVC383 | (20) |
| | | | | OvinS5 | |
| F17G | L33969 | 950 | 36 | O15: KRVC383 | (54) |
| | | | | OvinS5 | |
| F18 | M61713 | 510 | 37 | O139: K82 P88-1199 | (45) |
| F4 | M29374 | 601 | 38 | O149: K91 P97- | (72) |
| | | | | 2554B | |
| F41 | X14354 | 431 | 39 | O9: K30 B44s | (72) |
| F5 | M35282 | 450 | 40 | O9: K30 B44s | (72) |
| F6 | M35257 | 566 | 41 | O9: K-P81-603A | (72) |
| fimA | Z37500 | 331 | 42 | 3292 | (65) |
| group I | | | | | |
| fimA | Z37500 | 331 | 42 | O157: H7 EDL933 | (65) |
| group | | | | | |
| II | | | | | |
| fimH | AJ225176 | 508 | 43 | O157: H7 EDL933 | (54) |
| fliC | U47614 | 625 | 44 | O157: H7 E32511 | (34) |
| focG | S68237 | 359 | 45 | O4: K12 J96 | (54) |
| fyuA | Z38064 | 207 | 46 | 1195 | (3) |
| hlyA | M10133 | 500 | 47 | O4: K12 J96 | (89) |
| hlyC | M10133 | 556 | 48 | O4: K12 J96 | (9) |
| ibe10 | AF289032 | 170 | 49 | O18 H87-5480 | (44, 54) |
| iha | AF126104 | 827 | 50 | O157: H7 E32511 | (53) |
| invX | L18946 | 258 | 51 | H84 (EIEC) | This |
| | | | | | study |
| ipaC | X60777 | 500 | 52 | O157: H7 E32511 | This |
| | | | | | study |
| iroN | AF135597 | 668 | 53 | CP9 | (53, 79) |
| irp1 | AF091251 | 1689 | 54 | 1195 | (3) |
| irp2 | L18881 | 1241 | 55 | 1195 | (3) |
| iss | X52665 | 607 | 56 | 3292 | This |
| | | | | | study |
| iucD | M18968 | 778 | 57 | 4787 | (42) |
| iutA | X05874 | 300 | 58 | 4787 | (47) |
| katP | X89017 | 2125 | 59 | O157: H7 EDL933 | (14) |
| kfiB | X77617 | 501 | 60 | K5(F9) 3669 | This |
| | | | | | study |
| KpsMTI I | X53819 | 270 | 61 | K5(F9) 3669 | (54) |
| KpsMTI | AF007777 | 390 | 62 | 215 | (54) |
| II | | | | | |
| 17095 | AF074613 | 659 | 63 | O157: H7 EDL933 | (15, 64) |
| leoA | AF170971 | 501 | 64 | O149: K91 P97- | This |
| | | | | 2554B | study |
| lngA | AF004308 | 424 | 65 | PB-176P cfa-II | This |
| | | | | | study |
| lt | J01646 | 275 | 66 | O149: K91 P97- | (23, 33) |
| | | | | 2554B | |
| neuC | M84026 | 500 | 67 | O2: K1 U9/41 | This |
| | | | | | study |
| nfaE | S61970 | 537 | 68 | 31a | (54) |
| ompA | V00307 | 1422 | 69 | O4: K12 J96 | (77) |
| ompT | X06903 | 559 | 70 | O4: K12 J96 | (51) |
| paa | U82533 | 360 | 71 | O157: H7 STJ348 | This |
| | | | | | study |
| papAH | X61239 | 721 | 72 | O4: K12 J96 | (54) |
| papC | X61239 | 318 | 73 | 4787 | (62) |
| papEF | X61239 | 336 | 74 | O4: K12 J96 | (88) |
| PapG | M20146 | 461 | 75 | O4: K12 J96 | (67) |
| group I | | | | | |
| PapG | M20181 | 190 | 76 | IA2 | (48) |
| group | | | | | |
| II | | | | | |
| PapG | X61238 | 268 | 77 | O4: K12 J96 | (48) |
| group | | | | | |
| III | | | | | |
| pai | AF081286 | 922 | 78 | h140 8550 | (54) |
| rfbO9 | D43637 | 501 | 79 | O9: F6 K P81- | This |
| | | | | 603A | study |
| RfbO101 | X59852 | 500 | 80 | O101 h510a | This |
| | | | | | study |
| RfbO111 | AF078736 | 406 | 81 | O111 H87-5457 | (75) |
| RfbE | S83460 | 292 | 82 | O157: H7 EDL933 | (43) |
| O157 | | | | | |
| RfbE | S83460 | 259 | 83 | O157: H7 STJ348 | (75) |
| O157 | | | | | |
| H7 | | | | | |
| Rfc O4 | U39042 | 786 | 84 | O4: K12 J96 | (54) |
| rtx | AE005229 | 521 | 85 | O157: H7 EDL933 | This |
| | | | | | study |
| sfaDE | X16664 | 408 | 86 | 4787 | (62) |
| sfaA | X16664 | 500 | 87 | 4787 | This |
| | | | | | study |
| stah | M29255 | 201 | 88 | H-10407 | This |
| | | | | | study |
| stap | M58746 | 163 | 89 | O149: K91 P97- | (81) |
| | | | | 2554B | |
| stb | M35586 | 368 | 90 | O149: K91 P97- | (58) |
| | | | | 2554B | |
| stx1 | L04539 | 583 | 91 | O157: H7 EDL933 | (35) |
| stx2 | AF175707 | 779 | 92 | O157 KNIH317 | (35) |
| stxA I | M23980 | 502 | 93 | O157: H7 EDL933 | This |
| | | | | | study |
| stxA | Y10775 | 482 | 94 | O157: H7 EDL933 | This |
| II | | | | | study |
| stxB I | M23980 | 151 | 95 | O157: H7 EDL933 | This |
| | | | | | study |
| stx B | Y10775 | 211 | 96 | O157: H7 EDL933 | This |
| II | | | | | study |
| stxB | M36727 | 226 | 97 | O101 h510a | This |
| III | | | | | study |
| tir | AF045568 | 442 | 98 | RDEC-1B | This |
| group I | | | | | study |
| tir | AF070067 | 479 | 99 | O157: H7 EDL933 | This |
| group | | | | | study |
| II | | | | | |
| tir | AB036053 | 443 | 100 | O126: H6 E2348/69 | This |
| group | | | | | study |
| III | | | | | |
| traT | J01769 | 288 | 101 | 3292 | (54) |
| tsh | AF218073 | 640 | 102 | O78: K80 Av 89- | (26) |
| | | | | 7098(143) | |
| uidA | S69414 | 250 | 103 | O157: H7 EDL933 | (17) |
| uspA | AB027193 | 501 | 104 | h140 8550 | This |
| | | | | | study |

Note:
Amplicons were prepared using primers noted herein and strains noted above as source of template for PCR amplification.

TABLE 2

DNA sequences of primers
designed in this study.

| | | SEQ | | SEQ |
|---|---|---|---|---|
| | | ID | | ID |
| Gene | Forward | NO: | Reverse | NO: |
| afaE5 | GCGATCATGGCCGCGACC | 105 | CAACTCACCCAGTAGCC | 106 |
| | AGCA | | CCAGT | |
| cdt2 | GAAAGTAAATGGAATATA | 107 | TTTGTGTTGCCGCCGCT | 108 |
| | AATG | | GGTGAA | |
| cdt3 | GAAAGTAAATGGAATATA | 109 | TTTGTGTCGGTGCAGCA | 110 |
| | AATG | | GGGAAA | |
| cfaI | GGTGCAATGGCTCTGACC | 111 | GTCATTACAAGAGATAC | 112 |
| | ACA | | TACT | |
| cs1 | GCTCACACCATCAACACC | 113 | CGTTGACTTAGTCAGGA | 114 |
| | GTT | | TAAT | |
| cs3 | GGGCCCACTCTAACCAAA | 115 | CGGTAATTACCTGAAAC | 116 |
| | GAA | | TAAA | |
| derb122 | CGTGTGGGAGCCCTGAGC | 117 | CCGGCCTGGTTGCTAGT | 118 |
| | CTT | | ATT | |
| espA | CATCAGTTGCTAGTGCGA | 119 | CAGCAAATGTCAAATAC | 120 |
| group | ATG | | GTT | |
| I | | | | |
| espA | CGACATCGACGATCTATG | 121 | CCAAGGGATATTGCTGA | 122 |
| group | ACT | | AATA | |
| II | | | | |
| espA | CATCAGTTGCTAGTGCGA | 123 | CAGCAAATGTCAAATAC | 124 |
| group | ATG | | GTT | |
| III | | | | |
| espB | CGGAGAGTACGACCGGCG | 125 | GCACGGCTGGCTGCTTT | 126 |
| group | CTT | | CGTT | |
| I | | | | |
| espB | GCTGCCATTAATAGCGCA | 127 | TATTGTTGTTACCAGCC | 128 |
| group | ACT | | TTGC | |
| II | | | | |
| espB | GTAATGACGGTTAATTCT | 129 | GCCGCATCAATAGCCTT | 130 |
| group | GTT | | AGAA | |
| III | | | | |
| espC | CCCATAACGGAACAACTC | 131 | CAGAATAGACCAAACAT | 132 |
| | AT | | CTGCA | |
| etpD | GGCCACTTTCAATGTTGG | 133 | CGACTGCACCTGTTCCT | 134 |
| | TCA | | GATTA | |
| invX | TCTGATATAGTTTATATG | 135 | TCAAACCCCACTCTTAA | 136 |
| | GGT | | TTAA | |
| ipaC | TTGCAAAAGCAATTTTGC | 137 | TGCCGAACAATGTTCTC | 138 |
| | AAC | | TGCA | |
| kfiB | AATTGTTTTAAAATCTGT | 139 | TGAGACTGAAATTACAT | 140 |
| | TCT | | TTAA | |
| leoA | GAACAATTCAAACAGTTC | 141 | TTATTCAAATCGCGCAA | 142 |
| | AGT | | TACC | |
| lngA | CAAATACAGTCCGCGTAC | 143 | CCATTGTTACCTAAAGA | 144 |
| | GA | | GCGT | |
| neuC | TTGGCAGTTACAGGAATG | 145 | AACAGTGAACCATATTT | 146 |
| | CAT | | TAGT | |
| paa | ATGAGGAACATAATGGCA | 147 | TCTGGTCAGGTCGTCAA | 148 |
| | GG | | TAC | |
| rfbO9 | GGTGATCGATTATTCCGC | 149 | ACGCCTCATCGGTCAGC | 150 |
| | TGA | | GCCT | |
| rfbO101 | TCTGCACGTTTAAAATTA | 151 | GTTTCTCCGTCAGAATC | 152 |
| | TTG | | AAGC | |
| rtx | CTACCGTAGCGGGCGATG | 153 | CAGCGCCTGTCCGTGTT | 154 |
| | GTA | | CGGC | |
| sfaA | CCCTGACCTTGGGTGTTG | 155 | GTACTGAACTTTAAAGG | 156 |
| | CGA | | TGG | |
| stah | AAGAAATCAATATTATTT | 157 | AATAGCACCCGGTACAA | 158 |
| | AT | | G | |
| stxA I | GCGAAGGAATTTACCTTA | 159 | CAGCTGTCACAGTAACA | 160 |
| | GA | | AAC | |
| stxA II | CTTGAACATATATCTCAG | 161 | ACAGGAGCAGTTTCAGA | 162 |
| | GG | | CAGT | |
| stxB I | GGTGGAGTATACAAAATA | 163 | ATGACAGGCATTAGTTT | 164 |
| | TAA | | TAAT | |
| stx B | TTCTGTTAATGCAATGGC | 165 | TTCAGCAAATCCGGAGC | 166 |
| II | GG | | CTGA | |
| stxB | GAAGAAGATGTTTATAGC | 167 | ACTGCAGGTATTAGATA | 168 |
| III | GG | | TGAT | |
| tir | ATTGGTGCCGGTGTTACT | 169 | CTCCCATACCTAAACGC | 170 |
| group | GCTG | | AAT | |
| I | | | | |
| tir | ATTGGTGTTGCCGTCACC | 171 | ACGCCATGACATGGGAG | 172 |
| group | GCT | | G | |
| II | | | | |
| tir | ATTGGTGCTGGTGTAACG | 173 | ATTGCGTTTAGGTATGG | 174 |
| group | ACT | | G | |
| III | | | | |
| uspA | CTACTGTTCCCGAGTAGT | 175 | GGTGCCGTCCGGAATCG | 176 |
| | GTG | | GCGT | |

TABLE 3
Pathotype grouping of E. coli virulence genes

| Pathotype | Pathotype-specific virulence genes |
|---|---|
| UPEC | sfaA; sfaDE; clpG; iutA; nfaE; pai; iroN; |
| | cvaC; kpsMT2; kpsMT3; hlyA; hlyC; focG; |
| | afaD8; bmaE; cs31A; drb122; kfiB; afa3; |
| | afa5; afaE7; papEF; papC; papGI; papGII; |
| | papGII; papAH |
| ETEC | lngA; sth; stp; stb; It; F18; F41; leoA; |
| | rfbO101; F5; F6; F17A; F17G; cfaI; csl; |
| | cs3; F4 |
| EPEC | bfpA; eaf; espC |
| EHEC | ehxA; etpD; katP; L9075; rfbEO157; |
| | rfbO111; rfbO157H7; rtx; stx1; stx2; |
| | stxA1; stxA2;; StxB1; StxB2; Stx3A |
| EPEC and | eae; espP; espA1; espA2; espA3; paa; |
| EHEC (i.e. | espB1; espB2; espB3; tir1; tir2; tir3; |
| common to | espC |
| both) | |
| DAEC | aida |
| EAEC | aggA; aggC |
| EIEC | ipaC;. invX |
| CDEC | cdt1; cdt2; cdt3; cnf1; cnf2 |
| MENEC | rfcO4; iucD; ibe10; neuC; nfbO9 |

Throughout this application, various references are referred to describe more fully the state of the art to which this invention pertains. The disclosures of these references are hereby incorporated by reference into the present disclosure.

### REFERENCES

1. An, H., et al. (1999) Adv Exp Med Biol. 473:179-84.

2. Anderson, J. D., et al. (1987) Pediatr Infect Dis J. 6:1135-6.

3. Bach, S., et al. (2000) FEMS Microbiol Lett. 183:289-94.

4. Beaudry, M., et al. (1996) J Clin Microbiol. 34:144-8.

5. Benz, I., et al. (1992) Mol Microbiol. 6:1539-46.

6. Bertani, G. (1951) J. Bacteriol. 62:293-300.

7. Bertin, Y., et al. (1998) FEMS Microbiol Lett. 162:235-9

8. Beutin, L. (1999) Vet Res. 30:285-98.

9. Bingen, E., et al. (1998) J Infect Dis. 177:642-50.

10. Bloch, C. A., et al. (1996) Infect Immun. 64:3218-23.

11. Blum, G., et al. (1994) Infect Immun. 62:606-14.

12. Boyd, E. F., et al. (1998) J Bacteriol. 180:1159-65.

13. Brunder, W., et al. (1999) Microbiology. 145:1005-14.

14. Brunder, W., et al. (1996) Microbiology. 142:3305-15.

15. Burland, V., et al. (1998) Nucleic Acids Res. 26:4196-204.

16. Call, D. R., et al. (2001) Int J Food Microbiol. 67:71-80.

17. Cebula, T. A., et al. (1995) J Clin Microbiol. 33:248-50.

18. Chizhikov, V., et al. (2001) Appl Environ Microbiol. 67:3258-63.

19. Cho, J. C., et al. (2001) Appl Environ Microbiol. 67:3677-82.

20. Cid, D., et al. (1999) J Clin Microbiol. 37:1370-5.

21. Clegg, S. (1982) Infect Immun. 38:739-44.

22. Daigle, F., et al. (1994) Can J Microbiol. 40:286-91.

23. Dallas, W. S., et al. (1979) J Bacteriol. 139:850-8.

24. De Boer, E., et al. (1999) Int J Food Microbiol. 50:119-30.

25. Dozois, C. M., et al. (1997) FEMS Microbiol Lett. 152:307-12.

26. Dozois, C. M., et al. (2000) Infect Immun. 68:4145-54.

27. Elliott, S. J., et al. (1998) Mol Microbiol. 28:1-4.

28. Evans, D. G., et al. (1978) Infect Immun. 21:638-47.

29. Evans, D. G., et al. (1977) J Infect Dis. 136 Suppl:S118-23.

30. Evans, D. J., Jr., et al. (1976) J Infect Dis. 133 Suppl:97-102.

31. Feng, P., et al. (2000) Mol Cell Probes. 14:333-7.

32. Franke, J., et al. (1994) J Clin Microbiol. 32:2460-3.

33. Furrer, B., et al. (1990) Lett Appl Microbiol. 10:31-4.

34. Gannon, V. P., et al. (1997) J Clin Microbiol. 35:656-62.

35. Gannon, V. P., et al. (1992) Appl Environ Microbiol. 58:3809-15.

36. Garcia, E., et al. (1988) Antonie Van Leeuwenhoek. 54:149-63.

37. Girardeau, J. P., et al. (1991) J Bacteriol. 173:7673-83.

38. Groisman, E. A., et al. (1996) Cell. 87:791-4.

39. Gunzburg, S. T., et al. (1995) J Clin Microbiol. 33:1375-7.

40. Hacker, J., et al. (1990) Microb Pathog. 8:213-25.

41. Harel, J., et al. (1993) Vet Microbiol. 38:139-55.

42. Herrero, M., et al. (1988) J Bacteriol. 170:56-64.

43. Hu, Y., et al. (1999) J Appl Microbiol. 87:867-76.

44. Huang, S. H., et al. (1995) Infect Immun. 63:4470-5.

45. Imberechts, H., et al. (1992) Infect Immun. 60:1963-71.

46. Ito, H., et al. (1990) Microb Pathog. 8:47-60.

47. Johnson, J. R., et al. (1998) J Infect Dis. 177.

48. Johnson, J. R., et al. (1996) J Infect Dis. 173:920-6.

49. Johnson, J. R., et al. (2001) J Infect Dis. 183:78-88.

50. Johnson, J. R., et al. (2001) J Infect Dis. 183:1508-17.

51. Johnson, J. R., et al. (2000) Infect Immun. 68:3327-36.

52. Johnson, J. R., et al. (2002) J Infect Dis. 185:774-84.

53. Johnson, J. R., et al. (2000) Infect Immun. 68:3040-7.

54. Johnson, J. R., et al. (2000) J Infect Dis. 181:261-72.

55. Johnson, J. R., et al. (2001) J Infect Dis. 183:897-906.

56. Johnson, W. M., et al. (1988) Microb Pathog. 4:103-13.

57. Johnson, W. M., et al. (1987) FEMS Microbiol Lett. 43:19-23.

58. Knutton, S., et al. (1987) Infect Immun. 55:69-77.

59. Kuhnert, P., et al. (2000) FEMS Microbiol Rev. 24:107-17.

60. Kurazono, H., et al. (2000) Microb Pathog. 28:183-9.

61. Lalioui, L., et al. (1999) Infect Immun. 67:5048-59.

62. Le Bouguenec, C., et al. (1992) J Clin Microbiol. 30:1189-93.

63. Li, J., et al. (2001) J Clin Microbiol. 39:696-704.

64. Makino, S., et al. (1999) Epidemiol Infect. 123:25-30.

65. Marc, D., et al. (1996) J Med Microbiol. 44:444-52.

66. Martin, C., et al. (1997) Res Microbiol. 148:55-64.

67. Mitsumori K, T. A. et al. (1998) FEMS Immunol Med Microbiol. 21:261-8.

68. Moseley, S. L., et al. (1983) J Bacteriol. 156:441-3.

69. Muhldorfer, I., et al.(1994) Microb Pathog. 16:171-81.

70. Murray, A. E., et al. (2001) Proc Natl Acad Sci U S A. 98:9853-8.

71. Nataro, J. P., et al. (1998) Clin Microbiol Rev. 11:142-201.

72. Ojeniyi, B. et al. (1994) Zentralbl Veterinarmed [B]. 41:49-59.

73. Orndorff, P. E., et al. (1984) J Bacteriol. 159:736-44.

74. Oswald, E., et al. (1994) J Med Microbiol. 40:428-34.

75. Paton, A. W., et al. (1998) J Clin Microbiol. 36:598-602.

76. Perna, N. T., et al. (2001) Nature. 409:529-33.

77. Pfaff-McDonough, S. J., et al. (2000) Avian Dis. 44:23-33.

78. Rich, C., et al. (1999) FEMS Microbiol Lett. 173:55-61.

79. Russo, T. A., et al. (1999) Infect Immun. 67:5306-14.

80. Sandhu, K. S., et al. (1997) Adv Exp Med Biol. 412:295-302.

81. Savarino, S. J., et al. (1993) Proc Natl Acad Sci U S A. 90:3093-7.

82. Schmidt, H., et al. (1997) FEMS Microbiol Lett. 148:265-72.

83. Starcic, M., et al. (2002) Vet Microbiol. 85:361-77.

84. Strockbine, N. A., et al. (1988) J Bacteriol. 170:1116-22.

85. Wasteson, Y., et al. (1988) J Clin Microbiol. 26:2564-6.

86. Welch, R. A., et al. (1983) Infect Immun. 42:178-86.

87. Wilfert, C. M. (1978) Annu Rev Med. 29:129-36.

88. Yamamoto S, et al. (1995) FEMS Immunol Med Microbiol. 12:85-90.

89. Yamamoto, S. et al. (1995) FEMS Immunol Med Microbiol. 12:85-90.

90. Yamamoto, T. et al. (1996) Infect Immun. 64:1441-5.

91. Yamasaki, S. et al. (1996) Microbiol Immunol. 40:345-52.

92. Zhu, C. et al. (2001) Infect Immun. 69:2107-15.

What is claimed is:

1. An array comprising:
(a) a substrate; and
(b) a plurality of nucleic acid probes, each of said probes being bound to said substrate at a discrete location;
said plurality of probes comprising a first probe for a first pathotype of a species of a microorganism and a second probe for a second pathotype of said species, wherein said first and second pathotypes are not identical.

2. The array of claim 1, comprising at least two probes for a single pathotype, wherein said two probes are not identical.

3. The array of claim 2 wherein said array comprises a subarray, wherein said subarray comprises said at least two probes at adjacent discrete locations on said substrate.

4. The array of claim 1 wherein said probe is for a virulence gene or fragment thereof or a sequence substantially identical thereto, wherein said virulence gene is associated with pathogenicity of said microorganism.

5. The array of claim 1, wherein said microorganism is a bacterium.

6. The array of claim 5, wherein said bacterium is of the family Enterobacteriaceae.

7. The array of claim 6, wherein said bacterium is E. coli.

8. The array of claim 7, wherein said first and second pathotypes each independently comprise a pathotype selected from the group consisting of:
(a) enterotoxigenic E. coli (ETEC);
(b) enteropathogenic E. coli (EPEC);
(c) enterohemorrhagic E. coli (EHEC);
(d) enteroaggregative E. coli (EAEC);
(e) enteroinvasive E. coli (EIEC);
(f) uropathogenic strains (UPEC);
(g) E. coli strains involved in neonatal meningitis (MENEC);
(h) E. coli strains involved in septicemia (SEPEC);
(i) cell-detaching E. coli (CDEC); and
(j) diffusely adherent E. coli (DAEC).

9. The array of claim 7, wherein said first pathotype is selected from the group consisting of:
(a) enteroaggregative E. coli (EAEC);
(b) enteroinvasive E. coli (EIEC);
(c) E. coli strains involved in neonatal meningitis (MENEC);
(d) E. coli strains involved in septicemia (SEPEC);
(e) cell-detaching E. coli (CDEC); and
(f) diffusely adherent E. coli (DAEC).

10. The array of claim 4, wherein said virulence gene encodes a polypeptide of a class of proteins selected from the group consisting of toxins, adhesion factors, secretory system proteins, capsule antigens, somatic antigens, flagellar antigens, invasins, autotransporter proteins, and aerobactin system proteins.

11. The array of claim 4, wherein said virulence gene is selected from the group consisting of afaBC3, afaE5, afaE7, afaD8, aggA, aggC, aida, bfpA, bmaE, cdt1, cdt2, cdt3, cfaI, clpG, cnf1, cnf2, cs1, cs3, cs31a, cvaC, derb122, eae, eaf, east1, ehxA, espA group I, espA group II, espA group III, espB group I, espB group II, espB group III, espC, espP, etpD,F17A, F17G, F18, F4, F41, F5, F6, fimA group I, fimA group II, fimH, fliC, focG, fyuA, hlyA, hlyC, ibe10, iha, invX, ipaC, iroN, irp1, irp2, iss, iucD, iutA, katP, kfiB, kpsMTII, kpsMTIII, 17095, leoA, lngA, lt, neuC, nfaE, ompA, ompT, paa, papAH, papC, papEF, papG group I, papG group II, papG group III, pai, rfbO9, rfbO101, rfbO111, rfbE O157, rfbE O157 H7, rfc O4, rtx, sfaDE, sfaA, stah, stap, stb, stx1, stx2, stxA I, stxA II, stxB I, stx B II, stxB III, tir group I, tir group II, tir group III, traT, and tsh.

12. The array of claim 1 wherein said probe comprises a nucleic acid sequence selected from the group consisting of SEQ ID NO:1 to SEQ ID NO:102, or a fragment thereof, or a sequence substantially identical thereto.

13. A method of detecting the presence of a microorganism in a sample, said method comprising:
(a) contacting the array of claim 1 with a sample nucleic acid of said sample; and
(b) detecting association of said sample nucleic acid to a probe on said array;
wherein association of said sample nucleic acid with said probe is indicative that said sample comprises a microorganism from which the nucleic acid sequence of said probe is derived.

14. The method of claim 13, wherein said method further comprises extracting said sample nucleic acid from said sample prior to contacting it with said array.

15. The method of claim 13, wherein said sample nucleic acid is not amplified by PCR prior to contacting it with said array.

16. The method of claim 13, wherein said method further comprises digesting said sample nucleic acid with a restriction endonuclease to produce fragments of said sample nucleic acid.

17. The method of claim 16, wherein said fragments are of an average size of about 0.2 Kb to about 12 Kb.

18. The method of claim 13, wherein said sample is selected from the group consisting of environmental samples, biological samples and food.

19. The method of claim 18 wherein said environmental samples are selected from the group consisting of water, air and soil.

20. The method of claim 18 wherein said biological samples are selected from the group consisting of blood, urine, amniotic fluid, feces, tissues, cells, cell cultures and biological secretions, excretions and discharge.

21. The method of claim 13, wherein said method is further for determining a pathotype of a species of said microorganism, wherein said probe is for a pathotype of said species and wherein association of said sample nucleic acid with said probe is indicative that said microorganism is of said pathotype.

22. The method of claim 13, wherein said sample is a tissue, body fluid, secretion or excretion from a subject and said method is further for diagnosing an infection by said microorganism in said subject, wherein association of said nucleic acid with said probe is indicative that said subject is infected by said microorganism.

23. The method of claim 22, wherein said method is for diagnosing a condition related to infection by said microorganism in said subject, wherein said probe is for a pathotype of said species and wherein association of said sample nucleic acid with said probe is indicative that said microorganism is of said pathotype and that said subject suffers from a condition associated with said pathotype.

24. The method of claim 23, wherein said condition is selected from the group consisting of: diarrhea, hemorrhagic colitis, hemolytic uremic syndrome, invasive intestinal infections, dysentery, urinary tract infections, neonatal meningitis and septicemia.

25. The method of claim 22, wherein said subject is a mammal.

26. The method of claim 22, wherein said subject is a human.

27. A commercial package comprising the array of claim 1 together with instructions for:
(a) detecting the presence of a microorganism in a sample;
(b) determining the pathotype of a microorganism in a sample;
(c) diagnosing an infection by a microorganism in a subject;
(d) diagnosing a condition related to infection by a microorganism, in a subject; or
(e) any combination of (a) to (d).

28. A method of producing an array for pathotyping a microorganism in a sample, said method comprising:
(a) providing a plurality of nucleic acid probes, said plurality of probes comprising a first probe for a first pathotype of a species of said microorganism and a second probe for a second pathotype of said species, wherein said first and second probes are different; and
(b) applying each of said plurality of probes to a different discrete location of a substrate.

29. A method of producing an array for pathotyping a microorganism in a sample, said method comprising:
(a) selecting a plurality of nucleic acid probes, said plurality of probes comprising a first probe for a first pathotype of a species of said microorganism and a second probe for a second pathotype of said species, wherein said first and second probes are different; and
(b) synthesizing each of said plurality of probes at a different discrete location of a substrate.


SEQUENCE LISTING (176 sequences)

SEQ ID NO: 1 - DNA, 793 bp, Escherichia coli
catcaagctgtttgttcgtccgccggcggtgaaggggcgaccggatgatgtggccggcaaggtggagtggcagagggccggcaacaggctgaagggggttaacccgacgccgttttacatcaacctgtccacgctgacggtggggggtaaggaagtgaaggagcgtgaatatattgcgccgttttcctcccgtgaatatccgctgcctgcggggcatcgggtaaggttcagtggaaggtgataacggattacggcgggaccagtaagcagtttgaggcagagctgaagggttgaatacataaggtgataacagggtaaatgacgggctgacagatgcgtgatacttcttcagggcggatgagaacgggggtgacagggctggcgctggctgtgatggtggcctgtgtgatgtttcgtgcggagagtggtattgcgcgcacctactcctttgatgcggccatgctgaaaggtggcgggaagggggtggacctgaccctgtttgaggaaggtgggcagttacccggcatttatccggttgacattatcctgaatggttcccgtgtggattcacaggagatggcctttcacgcggagagggacgcggagggcaggccttatctgaagacctgtctgacccgtgagatgctggcgcgttacggggtcaggattgaggaatatccggcgttgttccgtgcatccggagagggtcgtggtgcctccgtggcggaggaggcctgtgctgacctgacggcgataccgcaggccacggagagttatcagtttgctgcccagc

SEQ ID NO: 2 - DNA, 470 bp, Escherichia coli
gcgatcatggccgcgaccagcactatcctcgcgatgagctcctcgcatgcagcgttcacaggaagtggtagcaccggtacgacaaaactaaccgttaccgaacagtgccaagtgctggtcaccggatctgacgtcaccaaaacgcgcggagaactcaccgacggggcccgtgtgggggtcctgtccgtaaccgcaaaaggctgtaacaccgagcatgcagcgttgcgtgcacagccagacaactaccaccagggcaagatcgtactgatccgcgatgactatcaggcacggataaatgtccgcttgcaggccaccgacgggcgtgcgtggaataccaacggcgacaccgtataccgcgccgatgctgggaactggggtggcagcttgttcgtagtcgtggacggggacaacgtggacaaaccgaccgggtcctacacactgaacctggactggggctactgggtgagttg

SEQ ID NO: 3 - DNA, 618 bp, Escherichia coli
gctaaatcaactgttgatgttgcaacggattctgttgatacatcctttatcatccgggacgactgtgcgatttctgttactgcgcagtctccaaaaacgtttaccttgagtgaagtcaaaaatcatgttagagcagcagatattaccattacacctacatgtggtggtaaatatttatgggcagaaatgaaagaggttgactctcagggatttggtattgcgagaactgatgctggtgatcttgcttctattacttgggtacaggatgggaattgggatgctggtgaaggtgagagagtggctaagacaaccctaccgaccgtggcttctcaaggggttccttatcctgcagtcttcgtaactcaggggagtacgggttaccgaagaaagctggagaatacaaatttggcctcactgttggctattgggtggagtgagttagaccgcaaaaattagtgttctctacaaagcatactgattgattaactttcaaggaagttcattatgcagggatgcagtcaagtgcaaaatcgtatcgtgattgttaccaacagtgtgaaagtgactcttctgttagcgccagtgatataagacggtaattcgccatttggattgtcc

SEQ ID NO: 4 - DNA, 351 bp, Escherichia coli
gttgaactgagtcttaataccagtgatggaaggagtggcgagttaaaagacggtacgaaggtggcaacaggaaggattatctgccgaggcacctatacaagttttcatatctggatgaatagcagacaaatgggaaatattcctggtcactatattatactgggtagacatgacagtcataatgaaatgcgggttaggctggatggcgcaggatggttgccatcggtaagtgatgggcaaggtatggtcagtaccgggatacctgagcagcatacatttgatgttgtgattgacggaaatcagctgcttgggcctgatgaatatatattatcagttagcggagaatgctca

SEQ ID NO: 5 - DNA, 432 bp, Escherichia coli
gcgttagaaagacctccaataaaagcaactgagacaatccgcctcaccgttacaaatgattgtcctgttactatagctacaaatagtccaccaaatgttggtgtatcgtcaacaacaccaataatatttaacgcaacagtaacgacgacagagcaatgtgctaaaagcggtgcaagggtctggttatggggaacaggtgccgctaataagtgggtcctagagcatactacaaatacaaaacaaaaatacacattaaatccatctatagatggaaattcatatttccagactccaggaactaatgcagcaatttataaaaatgtgacaaccagagacagagttctgaaggcaagtgtcaaggttgaccctaaaattcaagtattaataccaggcgaatatagaatgatactccatgccggaattaatttttaa

SEQ ID NO: 6 - DNA, 528 bp, Escherichia coli
tattaaaccatggtagcggggggatagacttaactctacttgagaaaggagggcagttgcctggtatttatccggttgatataattttaaatggttcgcgtattgattcaagggatatattcttttacacaaaaaaaaataggcatggtgaatattacctgaaaccctgtttaactcgagatattttgattaattacggagtaaaaacagaagaataccctaatcttttccggcaaaatagtgaaaaaaatagagacagtagcgattgtgctgacttatcagtgatcccccaagctacagaagactatcattttataaaacagcaactaatactcggaattccacaagttgcgatccgcccaccattgacaggcattgcccatgaaacaatgtgggatgatggtatatcagcatttttgttgaactggcaagtagaggggagtcattgggagtatagaagtaatactcgaaattcttcagacaatttttgggccagtttggaacctggaatcaatctcggatcttggc

SEQ ID NO: 7 - DNA, 586 bp, Escherichia coli
acagtatcatatggagccactccagacaggcctggattgtggcctcagagttagccagaggacatggttttgtccttgcaaaaaatacactgctggtattggcggttgtttccacaatcggaaatgcatttgcagtaaatatttcaggcacagtatcttcaggaggaactgtttcttccggcgaaacacaaatcgtgtattccggtcggggaaacagtaatgccactgtaaatagtggaggaacacaaatcgtcaataatggtgggaaaaccactgctacaactgttaatagttcaggaagccagaacgtcgggacttcaggagcaacaataagcacaattgtcaattctggtggcattcagcgagtcagttcaggtggtgtggcctctgcaacaaatttaagtggcggggctcagaacatctataatcttggccatgcatcaaataccgttatttttagcggtggaaatcagacgattttttcaggaggtataactgatagtacaaatatcagctccggtggccaacagcgtgtcagtagtggtggcgttgcctcgaacaccaccattaatagttctggcgcaca

SEQ ID NO: 8 - DNA, 324 bp, Escherichia coli
aatggtgcttgcgcttgctgccaccgttaccgcaggtgtgatgttttactaccagtctgcgtctgattccaataagtcgcagaatgctatttcagaagtaatgagcgcaacgtctgcaattaatggtctgtatattgggcagaccagttatagtggattggactcaacgattttacttaacacatctgcaattccggataattacaaagatacaacaaacaaaaaaataaccaacccatttgggggggaattaaatgtaggtccagcaaacaataacaccgcatttggttactatctgacgcttaccaggttggataaagcggc

SEQ ID NO: 9 - DNA, 505 bp, Escherichia coli
atggcgctaacttgccatgctgtgacagtaacagccactcatacagttgaatcagatgctgaattcacaatagattgggtcgacgctgggccaacgactacagatgcaaaagatggtgaggtttgggggcaccttgatatgactcaaaccaggggaacaccaacattcggaaaactccgcaatcctcaaggagagacttcgccaggaccgttgaaggcgccattcagttttaccgggccagatggtcatactgcaagagcgtaccttgattcatacggcgcaccgattcacaactacgcaggggataaccttgctaatggggtgaaggtaggtagtggaagcggaaacactccatttgttgttgggacagcaagtcgactaactgcaagaatcttcggagaccagacattggttccaggagtctaccggacaacctttgaattaactacttggaccgactgacggaaaattaacctgatgaaagaagggggctatatgtccccct

SEQ ID NO: 10 - DNA, 412 bp, Escherichia coli
caatagtcgcccacaggagttgtttatatatttctcacgtgttgatgcattcgctaacagagtaaatcttgcgattgtttcaaacagaagagctgatgaggtgattgtattacctcctccaactgttgtatcacgaccgatcatcggcattagaattggtaatgatgttttcttctcaacccatgcattggcgaatcggggcgtggattcaggagcaattgtaaatagtgtttttgagttcttcaacagacaaacggatcctataagacaggccgctaactggatgattgcaggagattttaaccgttcaccggctacactattttcaactcttgaaccagggattcgcaatcatgtaaatattattgctccaccagatccaacgcaagccagtggtggtgttcttgattat

SEQ ID NO: 11 - DNA, 556 bp, Escherichia coli
gaaagtaaatggaatataaatgtccggcaattaatttctggtgaaaatgctgtagacattttagctgtacaagaggcaggctctccgccgtcaacggctgtagatacaggtacacttattccttccccaggaattcccgtccgagagcttatctggaacttgtcgacaaatagcaggccacagcaagtatatatatatttttccgctgttgatgccctcggtggaagagtcaatcttgctctggttagcaatcggcgggccgatgaagtgtttgttcttagtcctgtaagacaaggtggacgaccattgcttggcatacgaattggtaatgatgcatttttcactgcacacgccatagctatgcgaaacaatgatgccccggctcttgttgaggaagtgtataacttcttccgcgacagcagagacccagtacaccaggcgcttaactggatgattcttggtgatttcaaccgtgaacctgcggatttagagatgaaccttactgttcccgtaagaagggcatcagaaattatttcaccagcggcggcaacacaaa

SEQ ID NO: 12 - DNA, 556 bp, Escherichia coli
gaaagtaaatggaatataaatgtccgacaattaatttctggtgaaaatgccgtagatattttagctgtgcaggaggcaggttctccgccatcaacggctgtagatacaggtagagttattccttccccaggcattcctgtccgggagcttatctggaacttgtctacaaatagcagaccacagcaagtatatatatatttttctgctgttgatgcctttggtggaagggtcaatcttgctctggttagcaatcggcaggccgatgaagtgtttgttcttcgcccggtaaggcaaggtgggcggccattgcttggcatacggattggcaatgatgcatttttcactgcacatgcgatagctacgcgaaacaatgacgctcccgctcttgttgaagaagtctatagcttctttcgtgacagccgagacccagtccaccaggccattaactggatgattcttggtgattttaatcgcgaacctgatgatttagaggtgaaccttacagttcctgtaagaaatgcatcagaaattattttccctgctgcaccgacacaaa

SEQ ID NO: 13 - DNA, 479 bp, Escherichia coli
ggtgcaatggctctgaccacaatgtttgtagcagtgagtgcttcagcagtagagaaaaatattactgtaacagctagtgttgatcctgcaattgatcttttgcaagctgatggcaatgctctgccatcagctgtaaagttagcttattctcccgcatcaaaaacttttgaaagttacagagtaatgactcaagttcatacaaacgatgcaactaaaaaagtaattgttaaacttgctgatacaccacagcttacagatgttctgaattcaactgttcaaatgcctatcagtgtgtcatggggaggacaagtattatctacaacagccaaagaatttgaagctgctgctttgggatattctgcatccggtgtaaatggcgtatcatcttctcaagagttagtaattagcgctgcacctaaaactgccggtaccgccccaactgcaggaaactattcaggagtagtatctcttgtaatgac

SEQ ID NO: 14 - DNA, 403 bp, Escherichia coli
gggcgctctctccttcaacaacactatcaaggaaatgacaggtgacagtaagctgctgaccatcactcagtctgaaccagctcctattcttttagggcgcacaaaagaggcgtttgcagcatcgattgttggtgttggtgcaattcctttaattgcgttcagtgattatgaagggaacggagttgccttacagagttctggggataacggtaaggggttctttgaattgcccatgaaagatgatagtggaaataatctcggtagcgtaaaagttaatgttacttctgctggcctgttttcctatagtgaaatatcaacaggtttagttggtataacttctgttgccagtggcgataatacaagtatttattatggtggtctggtgtcgccagcaattagggcg

SEQ ID NO: 15 - DNA, 1112 bp, Escherichia coli
gggggaagtacagaagaattacacgaaattttgttaggtcagggcccacagtcaagcttaggttttactgaatatacctcaaatgttaacagtgcagatgcagcaagcagacgacactttctggtagttataaaagtgcacgtaaaatatatcaccaataataatgtttcatatgttaatcattgggcaattcctgatgaagccccggttgaagtactggctgtggttgacaggagatttaattttcctgagccatcaacgcctcctgatatatcaaccatacgtaaattgttatctctacgatattttaaagaaagtatcgaaagcacctccaaatctaactttcagaaattaagtcgcggtaatattgatgtgcttaaaggacggggaagtatttcatcgacacgtcagcgtgcaatctatccgtattttgaagccgctaatgctgatgagcaacaacctctctttttctacatcaaaaaagatcgctttgataaccatggctatgatcagtatttctatgataatacagtggggctaaatggtattccaacattgaacacctatactggggaaattccatcagactcatcttcactcggctcaacttattggaagaagtataatcttactaatgaaacaagcataattcgtgtgtcaaattctgctcgtggggcgaatggtattaaaatagcacttgaggaagtccaggagggtaaaccagtaatcattacaagcggaaatctaagtggttgtacgacaattgttgcccgaaaagaaggatatatttataaggtacatactggtacaacaaaatctttggctggatttaccagtactaccggggtgaaaaaagcagttgaagtacttgagctacttacaaaagaaccaatacctcgcgtggagggaataatgagcaatgatttcttagtcgattatctgtcggaaaattttgaagattcattaataacttactcatcatctgaaaaaaaaccagatagtcaaatcactattattcgtgataatgtttctgttttcccttacttccttgataatatacctgaacatggctttggtacatcggcgactgtactggtgagagtggacggcaa

SEQ ID NO: 16 - DNA, 1241 bp, Escherichia coli
tatcatacggcaggaggaagcaccgaagaattgcatgagattttgttggggcaaggcccacagtcgagtttaggttttactgaatatacttcaaatattaacagtgcagatgcggcaagcagacgacattttcttgtagtcataaaagtgcaagtgaaatatataaacaataataacgtttcgcatgttaatcactgggcaattcctgatgaggctccagtagaagtactggctgtggttgacaggagatttaatttccctgagccatcaactccacctaatatatcaattatacacaagttgttatctctgagatattttaaagaaaatatcgaaagtacatcaaggcttaacttacagaaattaaatcgtggtaatattgatatatttaaagggagggggagtatttcatcaacacgtcagcgtgcgatttatccgtattttgaatctgctaatgctgatgagcaacaacctgtctttttctacatcaaaaaaaaccggtttgatgactttggctatgatcaatatttctataatagtacagtggggttgaatggtattcccacattgaacacctatactggagaaattctatcagacgcatcctcgctcggctcaacttattggaaaaagtataatctcactaatgaaacaagcatcattcgtgtatcaaattctgctcgaggggcaaatggtataaaaatagcacttgaagaagtgcaggaaggtaaaccggtaatcattacaagcggaaatttgagcggttgtacaacaattgttgctcgaaaaggaggatacctttataaggtacatacaggtacaacaatacctttagctggttttacaagtacaacaggggtaaaaaaagctgtagaagtttttgaattacttacaaataatccaatgccgcgcgtagagggagtaatgaataatgattttttggtaaattatctggcggaaagttttgatgagtctttaataacgtactcatcatctgaacaaaaaataggtagtaagattactatttctcgcgacaatgtttctacttttccttactttcttgataacataccagaaaaaggctttggtacatcggtgactatattggtaagagtagatggtaatgttatcgtaaaatccttatctgagagttattctttaaatgtagaaaactccaatatatcagtattgcatgttttttcaaaagatttttgattcggaaaattattgtctattgtgac

SEQ ID NO: 17 - DNA, 321 bp, Escherichia coli
gctcacaccatcaacaccgttgttcatacaaatgactcagataaaggtgttgttgtgaagctgtcagcagatccagtcctgtccaatgttctgaatccaaccctgcaaattcctgtttctgtgaatttcgcaggaaaaccactgagcacaacaggcattaccatcgactccaatgatctgaactttgcttcgagtggtgttaataaagtttcttctacgcagaaactttcaatccatgcagatgctactcgggtaactggcggcgcactaacagctggtcaatatcagggactcgtatcaattatcctgactaagtcaacg

SEQ ID NO: 18 - DNA, 401 bp, Escherichia coli
gggcccactctaaccaaagaactggcattaaatgtgctttctcctgcagctctggatgcaacttgggctcctcaggataatttaacattatccaatactggcgtttctaatactttggtgggtgttttgactctttcaaataccagtattgatacagttagcattgcgagtacaagtgtttctgatacatctaagaatggtacagtaacttttgcacatgagacaaataactctgctagctttgccaccaccatttcaacagataatgccaacattacgttggataaaaatgctggaaatacgattgttaaaactacaaatgggagtcagttgccaactaatttaccacttaagtttattaccactgaaggtaacgaacatttagtttcaggtaattaccg

SEQ ID NO: 19 - DNA, 731 bp, Escherichia coli
gcacaattactgctgatgcgtataaagacaaatgggaatggatggttgggggcgctctctccttcaacaacactatcaaggaaatgacaggtgacagtaagctgctgaccatcactcagtctgaaccagctcctattcttttagggcgcacaaaagaggcgtttgcagcatcgattgttggtgttggtgcaattcctttaattgcgttcagtgattatgaagggaaaggagttgccttacagagttctggggataacggtaaggggttctttgaattgcccatgaaagatgatagtggaaataatctcggtagcgtaaaagttaatgttacttctgctggcctgttttcctatagtgaaatatcaacaggtttagttggtataacttctgttgccagtggcgataatacaagtatttattatggtggtctggtgtcgccagcaattagggcgggtaaagacgcagcatcagctgtgtcgaaatttggcaactataatcatacacaattgctgggccagcttcaagcagtaaaccctaacgcgggcaatagaggacaagtaaataaaaatagtgcggtctcacaaaatatggtgatgactactggtgatgtaattgcatcctcttacgcacttggtattgaccagggacagactattgaagcaacctttactaatcctgtggttagcaccacccagtggagtgctccgctgaacgtggcagtaacttataac

SEQ ID NO: 20 - DNA, 677 bp, Escherichia coli
cacacacaaacgggagctgtttgtagcgaagccactcgttcaaatcaattctcttgacgtggggaaatccgttttccaagcggaccccttatagggggttgagggcctcctacccttcactcttgactatgttaacgataatcattatcgttagtgtttgtgtggtaatgggatagaaagtaatgggataaaaagtaatggatagaaaaagaacaaaattagagttgttatttgcatttataataaatgccaccgcaatatatattgcattagctatatatgattgtgtttttagaggaaaggactttttatccatgcatacattttgcttctctgcattaatgtctgcaatatgttactttgttggtgataattattattcaatatccgataagataaaaaggagatcatatgagaactctgactctaaatgaattagattctgtttctggtggtgcttcagggcgtgatattgcgatggctataggaacactatccgggcaatttgttgcaggaggaattggagcagctgctgggggtgtggctggaggtgcaatatatgactatgcatccactcacaaacctaatcctgcaatgtctccatccggtttagggggaacaattaagcaaaaacccgaagggataccttcagaagcatggaactatgctgcgggaag

SEQ ID NO: 21 - DNA, 260 bp, Escherichia coli
cgtgtgggagccctgagccttaacgcgagagggtgtaacaccgagcatgcagcgctgcgcgctcaagcagacaactaccacaatggcaagatcgtactgctacgtgaagaccaacaagcgcggataaatgtgcgcttggtggcatccgatgggggccaatggactaacgacggcgcaaccacataccgcgacgctgccggggactggggcgggagcttgtacgtagtcgtggacggggacaatactagcaaccaggccgg

SEQ ID NO: 22 - DNA, 791 bp, Escherichia coli
cattatggaacggcagaggttaatctgcagagtggtaataactttgacggtagttcactggacttcttattaccgttctatgattccgaaaaaatgctggcatttggtcaggtcggagcgcgttacattgactcccgctttacggcaaatttaggtgcgggtcagcgttttttccttcctgaaaatatgttgggctataacgtcttcattgatcaggatttttctggtgataatacccgtttaggtattggtggcgaatactggcgagactatttcaaaagtagtattaacggctatttccgcatgagcggctggcatgagtcatacaataagaaagactatgatgagcgcccagcaaatggcttcgatatccgttttaatggctatctgccatcatacccggcattaggtgccaagctgatgtatgagcagtattatggtgataatgttgctttgtttaattctgataagctgcagtcgaatcctggtgcggcgaccgttggtgtaaactatactccgattcctctggtgacgatggggatcgattaccgtcatggtacgggtaatgaaaatgatctcctttactcaatgcagttccgttatcagtttgataaaccgtggtctcagcaaattgagccacaatatgttaacgagttaagaacattatcaggcagccgttacgatctggttcagcgtaataacaatattattctggagtacaaaaagcaggatattctttctctgaatattccgcatgatattaatggtactgaacgcagtacgcagaagat

SEQ ID NO: 23 - DNA, 397 bp, Escherichia coli
  misc_feature (224)..(224): n is a, c, g, or t
cagggtaaaagaaagatgataagttaacgcttggagtgatcgaacgggatccaaatcactgatcgatgttccatgcgaataagtgatcgatcatgtcggaatatccaaaaacccgaaatcaccagttgccacattgaacggcgctggtgatttcgggttcgtcactttatggataccatcaacccattccccggagaaagtatgggctggctaaagtgtagcgncattaagagcagttatttagtattttaatgagtatcgaatctttatatttgcatcattccgttgttggtccgccttctgacaagctgtgttggcagaagaaacgtcgttagcggttcctattttgttactacctagatatatatcaggtttttgataatacatggtccccata

SEQ ID NO: 24 - DNA, 117 bp, Escherichia coli
tcggatgccatcaacacagtatatccgaaggcccgcatccagttatgcatcgtgcatatggtgcacaacagcctgcgcttcgtgtcatggaaggactacaaagccgtcactcgcgac

SEQ ID NO: 25 - DNA, 159 bp, Escherichia coli
gtttattctggggcaggctcatcagaagtatttgctggtgaaggtcatgataccgtatcttataataagacggatgttggtaaactaacaattgatgcaacaggagcatcaaaacctggtgaatatatagtttcaaaaaatatgtatggtgacgtgaag

SEQ ID NO: 26 - DNA, 525 bp, Escherichia coli
ggatacatcaactgcaacatcagttgctagtgcgaacgcgagtacttcgacatcgacagtctatgacttaggcagtatgtcgaaagacgaagtagttcagctatttaataaagtcggtgttttcagtgcgcttctcatgtttgcctatatgtatcaggcacaaagcgatctgtcgattgcaaagtttgctgatatgaatgaggcatctaaggagtcaaccacagcccaaaaaatggctaatcttgtggatgctaaaattgctgatgttcagagtagttctgacaagaatgcaaaagccaaactacctaaagaagtgattgactatataaatgatcctcgcaatgacattacagtaagtggtattagcgatctaaatgctgaattaggcgctggtgatttgcaaacggtgaaggccgctatttcggccaaatcgaataacttgaccacggtagtgaataatagccagcttgaaatacagcaaatgtcaaatacgttaaacctattaacgagtgcacgttctgatat

SEQ ID NO: 27 - DNA, 523 bp, Escherichia coli
cgacatcgacgatctatgacttaggtaatatgtcgaaggatgaggtggttaagctatttgaggaactcggtgtttttcaggctgcgattctcatgttttcttatatgtatcaggcacaaagtaatctgtcgattgcaaagtttgctgatatgaatgaggcatctaaagcgtcaaccacggcacaaaagatggctaatcttgtggatgccaaaattgctgatgttcagagtagcactgataagaatgcgaaagccaaacttcctcaagacgtgattgactatataaacgatccacgtaatgacataagtgtaactggtattcgtgatcttagtggtgatttaagcgctggtgatctgcaaacagtgaaggcggctatttcagctaaagcgaataacctgacaacggtagtgaataatagccagctcgaaattcagcaaatgtcgaatacattaaatctcttaacgagtgcacgttctgatgtgcaatctctacaatatagaactatttcagcaatatcccttgg

SEQ ID NO: 28 - DNA, 487 bp, Escherichia coli
gggaacatgtcgaaagatgaagttgttgagctgtttaaaaaagttggcgtatttcaggctgcgcttatcatgtttgcttatatgtaccaggcacaaagcgagctatctattgctacatatgcagacatgaatgagtcatctaaggaatccaccgaggcacaaaaaatggccaatttggtggatgccaagatcgctgaagttcagtctagttcggaaaaggataaaaaggtcaaacttcctgacgaagtaattagttatattcaagattcacgaaatgggatttccgtaagtagtgatattgacatcaccaaagagttgggtgctggtgacctgcaaaccgtaaaagctgctatttcagcaaaagcaaataacctgacaacgacggtgaataataaccagcttacgttgcagcaaatgtctaatacgctgaatttattaacaaatgctcgttcagatatgcagtcgttgcaatatcgaactattcagg

SEQ ID NO: 29 - DNA, 502 bp, Escherichia coli
cggagagtacgaccggcgcttccagtgcagttgccgcatctgctttatcaattgattcatctctgcttactgatggtaaggttgatatttgtaagctgatgctggaaattcaaaaactcctcggcaagatggtgactctattgcaggattaccaacaaaaacaattggcgcaaagctatcagattcagcaggccgtttttgagagccagaataaagctattgaggaaaaaaaagccgcggcaaccgctgctttggttggcgggattatttcatcagcattggggatcttaggttcttttgcagcaatgaacaacgcggctaaaggggctggtgagattgctgaaaaagcaagctctgcatcttcaaaggctgctggtgcggcttctgaggttgcaaataaagctctggtcaaggctacggaaagtgttgctgatgtcgcagaggaggcatccagtgcgatgcagaaagcgatggccacaacaacgaaagcagccagccgtgc

SEQ ID NO: 30 - DNA, 377 bp, Escherichia coli
gctgccattaatagcgcaactaaaggcgcgagtgatgtcgctcagcaagccgcttctacttctgcgaagtctatcggtacagtctctgaagcttcaactaaagcactggcgaaggcttccgaaggtattgcagatgcagcagatgatgcagctggcgcaatgcagcaaactatcgcgacagctgcaaaagcggccagtcgtacatccggtatcactgatgatgttgctacttcggctcagaaagcttctcaggtagctgaagaggctgctgatgctgctcaagaattagcacagaaggcaggattattaagtcgctttactgctgctgccggaaggatttccggttcaacgccatttattgttgttaccagccttgc

SEQ ID NO: 31 - DNA, 395 bp, Escherichia coli
gtaatgacggttaattctgtttcggagaatactaccggctctaatgcaattaccgcatctgctattaattcatctttgcttaccgatggtaaggtcgatgtttctaaactgatgctggaaattcaaaaactcctgggcaagatggtgcgtatattgcaggattaccaacagcaacagttgtcgcagagctatcagatccaactggccgtttttgagagccagaataaagccattgatgaaaaaaaggccgctgcaacagccgctctggttggtggggctatttcatcagtattggggatcttaggctcttttgcagcaattaacagtgctacgaaaggcgcgagtgatattgctcaaaaaaccgcctctacatcttctaaggctattgatgcggc

SEQ ID NO: 32 - DNA, 500 bp, Escherichia coli
cccataacggaacaactcatcatgcaataagcacccaaaactggggacaaagctcatataaatatatagaccggatgacgaatggagattttgctgtaacacgacttgataagtttgttgttgaaacaacaggggtaaaaaattcagtagatttttctctcaatagtcatgatgctcttgaacgttatggtgtggagatcaatggtgagaaaaaaatcattggtttcagggttggggctgggacgacttataccgttcaaaatggtaatacatatagtacaggacaggtatacaatcctcttttgttaagcgcttcaatgtttcagttaaactgggataacaaaagaccatataataacacgacacctttttataatgaaactaccggtggagacagtggttccggtttctatctgtatgataacgtaaaaaaagaatgggttatgcttggtactttatttggaatagcatccagtggtgcagatgtttggtctattctg

SEQ ID NO: 33 - DNA, 1830 bp, Escherichia coli
aaacagcaggcacttgaacgttacggggttaattataaaggagaaaagaaacttatcgcattcagagccggctctggtgtggtatccgttaaaaaaaatggacgcataactccatttaatgaggtttcttataagccagaaatgttaaatggctctttcgttcacattgatgactggagtggatggctgatattaaccaacaaccagtttgatgagtttaataacattgcctctcagggtgacagcggttcagcactgttcgtctatgataaccaaaagaaaaagtgggttgtcgctggaactgtctgggggatttataattacgccaatggcaaaaaccacgcagcatacagtaaatggaaccagacaaccattgacaacctgaagaacaagtattcttacaacgtggatatgtcaggggctcaggttgcaaccattgaaaatggaaaactgacaggcactggctcagacaccaccgatataaaaaataaggacttaatatttactggcggtggagatatcctcctgaaatcctcttttgataatggtgctggcggtcttgtctttaatgataaaaagacctatcgagtaaacggggatgatttcacctttaaaggtgccggtgttgatacaagaaacggcagcaccgttgagtggaatatccggtatgataataaagacaaccttcacaaaattggtgatggcacattagatgtccgaaaaacccagaacaccaacctgaaaacaggtgagggtcttgtcattcttggagctgaaaaaacattcaataatatctacataaccagtggtgatggaactgtccgactgaatgcagaaaatgcactgtctggcggtgaatacaacggtattttctttgcgaaaaatggcggaactcttgacctgaacggatataatcagtctttcaataaaattgctgcaactgattcaggtgctgtaataaccaatacgtcaaccaaaaaatccattttatccctgaataatactgctgactatatctatcacggtaacataaacgggaatctggacgtacttcagcatcatgagacgaaaaaagagaaccgtcgtcttattcttgatgggggcgtggacacaacaaatgatataagcctgcgtaatacacaactgtccatgcagggacatgccactgaacatgccatttatcgggatggagctttctcttgttcactaccagctcctatgcgctttttgtgtggcagtgattatgttgcaggaatgcaaaatacagaagctgatgctgtaaaacaaaacggaaatgcctataaaaccaacaatgctgtctctgatttatcgcagccagactgggaaaccggaacattcagatttggaacgctacatcttgaaaattccgatttttctgttggtcgtaatgcaaatgtaatcggggacattcaggccagtaaatcaaacattactattggtgacactacagcatatattgatttgcatgctggtaaaaatattaccggtgatggttttggcttccgccagaatattgtgcgtggaaactcacaaggagaaacgctgtttacaggagggatcacagcagaagacagcactatcgttattaaagataaagcaaaagcattattttcaaattatgtatacctgctgaacacaaaagcaaccatagagaacggtgctgatgtgacaactcaaagtggtatgttctccacgagcgatatcagcatctctggtaatctgtccatgacaggcaatcccgacaaagacaataaattcgagccctcaatatatctgaatgatgcttcttatctactgactgacgactcc

SEQ ID NO: 34 - DNA, 499 bp, Escherichia coli
ggccactttcaatgttggtcaggaggtcccggtactttcgggctcacagacaacctctggggacaatatttttaacacggtcgagcgcaaaacggtggggatcaaactcagggtaaaaccccagatcaacgagggtgattccgtgttactggagatagaacaggaggtgtccggtgtggcggacactgcagtagccaccactactgacttgggagcaaccttcaacacccgaacagtgaccaatgccatgctggtcgggaatggcgaaacggtggtggtcggaggattactggataagtcgatcagggggagtgagagtaaagtgccactgctgggggatatcccggtactggggcatctttttcgcgcaaaaagcgaacagacagctaagcgtaatctgatgctgttcattcggccaactattattcgtgagcgcgacggatttcgtcatgcttcggccgaaaaataccagtcgtttaatcaggaacaggtgcagtcg

SEQ ID NO: 35 - DNA, 441 bp, Escherichia coli
atgcagaaaattcaatttatccttggaatactggcggctgcatcatcttctgctacgcttgcttatgacggtaaaattacttttaatggaaaagttgttgatcaaacttgttctgttacaacagaaagcaagaatttgacagttaagttaccaactgtctctgctaattcattagcttcaagcggaaaagtggtgggacttactcctttcacaattttgctggaagggtgcaatacgcctgccgtgacaggtgctcagaatgtaaatgcttatttcgaacctaatgcgaacacggattacaccactggtaatttaactaatacggcttcttctggtgcatctaatgttcagattcagctactgaatgcagatggggttaaagctattaaacttggtcaggctgctgcagctcagagtgtggatacagttgctattaatgat

SEQ ID NO: 36 - DNA, 950 bp, Escherichia coli
tgttggaccgtctcagggctcttattccagcactcatgcaatggataacctgccatttgtctataataccggttacaacattggatatcagaatgcaaatgtctggcgtattagtggcgggttttgtgttggtctggacgggaaagtggatttacccgtggttggcagtcttgacgggcagagtatttatgggctgacggaggaggtgggactccttatatggatgggggacacgaattattccaggggtaccgcgatgagtggaaactcatgggagaatgtcttttccggatggtgcgtgggaaattatgtatcaacgcagggactgtctgttcacgtaagaccggtaattttaaaaagaaattcctctgcgcaatacagtgtacagaaaaccagtatcgggagtatcagaatgaggccctataacggttcatctgcaggcagtgttcagaccacagtgaatttcagcctgaatccatttacgctgaatgacacagtaacatcgtgcagattactgacaccttccgcagtcaatgtcagcctggctgcaatttctgccggacaactaccatcatccggtgatgaagttgtcgccgggacaacatcactgaaattacagtgtgatgccggagtaacagtatgggcaacactgactgatgcgaccacaccgtccaacagaagcgatatactcacactgacgggggcatcgactgcaaccggagtcgggctgagaatatacaaaaacactgacagtacgcccctgaagtttggacctgattcgccggtaaagggaaatgaaaaccagtggcagttatcgacaggaacggaaacgtcaccctcagtccggttgtatgtaaagtatgtgaatactggtgagggaattaatccgggtacggttaacggaatatcaacatttacgttttcctatcagtaacagcgagttccgggag

SEQ ID NO: 37 - DNA, 510 bp, Escherichia coli
gtgaaaagactagtgtttatttcttttgttgcgctgtccatgacagcgggttccgcaatggctcagcaaggggatgttaaattctttggtaacgtatcagcaactacctgtaatttgacaccacaaataagtggcactgtaggagataccattcagcttggtactgttgcaccaagcggaactggtagtgaaattccttttgcactgaaggcttcttcaaatgttggcggttgtgcttccttgtccactaaaacagctgatataacttggagcgggcagttaaccgaaaaaggttttgctaatcaagggggggtggcaaatgattcatatgtcgctctgaaaaccgtgaacggtaaaacacaggggcaggaggttaaggcgtcgaatagcactgtaagtttcgatgcatcaaaagcaactacggaaggtttcaaatttactgctcaactgaaaggtggtcaaaccccgggtgacttccagggggcagcggcttacgcggttacttacaag

SEQ ID NO: 38 - DNA, 858 bp, Escherichia coli
atgaaaaagactctgattgcactggcaattgctgcatctgctgcatctggtatggcacatgcctggatgactggtgatttcaatggttcggtcgatatcggtggtagtatcactgcagatgattatcgtcagaaatgggaatggaaagttggtacaggtcttaatggatttggtaatgtattgaatgacctgaccaatggtggaaccaaactgaccattactgttactggtaataagccaattttgttaggccgaaccaaagaagcatttgctacgccagtaagtggtggtgtagatggaattcctcagattgcatttactgactatgaaggagcttctgtaaaactcagaaacactgatggtgaaactaataaaggtttagcatattttgttctgccgatgaaaaatgcagagggcactaaagttggttcagtgaaagtgaatgcatcttatgccggtgtgttcgggaaaggtggggttacttctgcggacggggagctgttttcgctttttgccgacgggttgcgcgctatcttttatggtggtttgacgacgactgtttcgggtgctgcactcacgagtgggagtgccgcagcggcgcgcacagagttgtttggaagtctatcaagaaatgatattctcggacagattcaaagagtaaacgcaaatattacttctcttgttgacgtcgcaggttcttacagggaagacatggagtacactgatggaactgttgtttctgctgcctatgcactgggtattgcaaacggtcagactattgaggcaacttttaatcaggctgtaactaccagcactcagtggagcgctccgctgaacgtagcaattacttattactaa

SEQ ID NO: 39 - DNA, 431 bp, Escherichia coli
gagggactttcatcttttagcaatactacaaatgaaattgttaaacggaagttgaatatttctgttccaacggatgaattatttttagcagcgaagatgagtgatgggattaaaggtgttttcgtagggaatacactcattcctaagattgaaatggcatcttatgatggtagtgttattacacctagtttcacttcaaatacagcaatggatattgctgtaaaagtaaaaaactcaggtgataatactgagctagggactctttctgttcctttgtcatttggtgcggcagttgcaactatttttgatggcgatactactgatagcgctgtagcgcatattatcggtggttctgctggtacagtatttgaagggcttgttaatccaggtcgatttactgatcagaatatagcctataaatggaatggact

SEQ ID NO: 40 - DNA, 450 bp, Escherichia coli
tgcgactaccaatgcttctgcgaatacaggtactattaacttcaatggcaaaataacgagtgctacttgtacaattgaccctgaggtcaatggtaatcgtacatcaactatagatcttgggcaggctgctattagtggtcatggcactgtagtggattttaaactaaaaccagcgcccggcagtaatgactgcctagcgaaaacaaatgctcgtattgactggtctggttctatgaacagtttaggttttaataatacagcttcaggaaatactgctgctaaaggataccatatgactttgcgcgcaacaaacgttggaaatgggtctggtggtgctaatattaatacttcattcactacggctgaatacactcacacttctgcaattcagtcatttaactattcagcccagctgaaaaaagatgaccgcgctccgtctaatggtggata

SEQ ID NO: 41 - DNA, 954 bp, Escherichia coli
aaatttagaaaagtgcattatgcttatcactagataagaaaataaaacacgaaatatagcgagccatatagcctgttgtgtttgtaatagataaaaaacacgcaattgattatttatgtatctttttgtttgtatttttttattaaaaaaagcacacaattactgcgtgcatcgaaatgagttgaagtggatgcatatatgcatgaaatgcttttaacttgaaagtcttaatgtttctattaattaagataaggtaatatgagaatgaaaaaatccgcattaacattagcagtgctttcctctctgttcagtggttactcgctcgcagcgcccgctgaaaacaacaccagccaggcaaatttagactttactggtaaagttactgccagtctatgccaagtggatacttctaatctgtcgcaaaccatagatcttggagagttgtctacttctgctcttaaagctactggcaaggggcctgccaagtcatttgcagttaatcttatcaactgcgatacaacattgaattctattaaatacactattgctggtaataataatacaggaagtgatactaaatatttagttccagcctccaatgatactagtgcatcaggagttggcgtatacattcaggacaacaacgcccaggctgtggaaattggtactgaaaaaactgtacctgtggtatcaaatggcggattagctctttcagaccaaagtattccactgcaagcatacatcggaaccaccacagggaatcctgatacaaacggtggagttacggccggtactgtcactgctagtgcagtaatgactattcgttcagcaggtacaccgtaattagataacaatttttatacaacaaaacaggaaggattttgaactaatccttcctgttattggagattgaaatgtctaagtttgtaatatttcttgtgtttttgtttatat

SEQ ID NO: 42 - DNA, 331 bp, Escherichia coli
gttgatcaaaccgttcagttaggacaggttcgtaccgccactttgaagcaggctggagcaaccagctctgctgtcggttttaacattcagctgaatgattgcgataccactgttgccacaaaagccgctgttgccttcttggggacggcgattgacagtactcatcctaaagtcctggctctacagagttcagctgcgggtagcgcaacaaacgttggcgtgcagattctggacagaacaggtaatgagctgacgctggacggtgcgacatttagtgcagaaacaaccctgaataacggtactaacaccattccgttccaggcgcgttatt

SEQ ID NO: 43 - DNA, 506 bp, Escherichia coli
tcgagaacggataagccgtggccggtggcgctttatttgacgcctgtgagcagtgcgggcggggtggcgattaaagctggctcattaattgccgtgcttattttgcgacagaccaacaactataacagcgatgatttccagtttgtgtagaatatttacgccaataatgatgtggtggtgcctactggcggctgcgatgtttctgctcgtgatgtcaccgttactctgccggactaccctggttcagtgccaattcctcttaccgtttattgtgcgaaaagccaaaacctggggtattacctctccggcacaaccgcagatgcgggcaactcgattttcaccaataccgcgtcgttttcacctgcacagggcgtcggcgtacagttgacgcgcaacggtacgattattccagcgaataacacggtatcgttaggagcagtagggacttcggcggtgagtctgggattaacggcaaattatgcacgtaccggagggcaggtgactgc

SEQ ID NO: 44 - DNA, 625 bp, Escherichia coli
gcgctgtcgagttctatcgagcgtctgtcttctggcttgcgtattaacagcgcgaaggatgacgccgcaggtcaggcgattgctaaccgttttacttctaacattaaaggcctgactcaggcggcccgtaacgccaacgacggtatttctgttgcgcagaccaccgaaggcgcgctgtccgaaatcaacaacaacttacagcgtattcgtgaactgacggttcaggccactacagggactaactccgattctgacctggactccatccaggacgaaatcaaatctcgtcttgatgaaattgaccgcgtatccggccagacccagttcaacggcgtgaacgtgctggcgaaagacggttcaatgaaaattcaggttggtgcgaatgacggcgaaaccatcacgatcgacctgaaaaaaatcgattctgatactctgggtctgaatggctttaacgtaaatggtaaaggtactattaccaacaaagctgcaacggtaagtgatttaacttctgctggcgcgaagttaaacaccacgacaggtctttatgatctgaaaaccgaaaataccttgttaactaccgatgctgcattcgataaattagggaatggcgataaagtcacagttg

SEQ ID NO: 45 - DNA, 359 bp, Escherichia coli
cagcacaggcagtggatacgacgattactgttacagggagggtattgccacgtacctgtaccattggtaatggaggaaacccaaacgccaccgttgttttggataacgcttacacttctgacctgatagcagccaacagcacctctcagtggaaaaatttttcgttgacattgacgaattgtcagaatgtaaacaatgttactagctttggtggaaccgcagaaaatacaaattattacagaaatacaggggatgctactaatatcatggttgagctacaggaacaaggtaatggtaatacccccttgaaagttggttcaacaaaagttgttacagtgagcaatgggcaggcgacattc

SEQ ID NO: 46 - DNA, 207 bp, Escherichia coli
ggcggcgtgcgcttctcgcatgataaatccagtacacaatatcacggcagcatgctcggcaacccgtttggcgaccagggtaagagcaatgacgatcaggtgctcgggcagctatccgcaggctatatgctgaccgatgactggagagtgtatacccgtgtagcccagggatataaaccttccgggtacaacatcgtgcctactgcg

SEQ ID NO: 47 - DNA, 500 bp, Escherichia coli
tgttgaaagatcagtcctcattacccagcaacattgggatacgctgataggtgagttagctggtgtcaccagaaatggagacaaaacactcagtggtaaaagttatattgactattatgaagaaggaaaacgtctggagaaaaaaccggatgaattccagaagcaagtctttgacccattgaaaggaaatattgacctttctgacagcaaatcttctacgttattgaaatttgttacgccattgttaactcccggtgaggaaattcgtgaaaggaggcagtccggaaaatatgaatatattaccgagttattagtcaagggtgttgataaatggacggtgaagggggttcaggacaaggggtctgtatatgattactctaacctgattcagcatgcatcagtcggtaataaccagtatcgggaaattcgtattgagtcacacctgggagacggggatgataaggtctttttatctgccggctcagccaatatctacgcag

SEQ ID NO: 48 - DNA, 556 bp, Escherichia coli
aggttcttgggcatgtatcctggctctgggccagttccccattacacagaaactggccagtctctttgtttgcaataaatgtattacctgcaatacgggctaaccaatatgctttattaacccgggataattaccctgttgcatattgtagttgggctaatttaagtttagaaaatgaaattaaatatcttaatgatgttacttcattagtcgcagaagactggacttctggtgatcgtaaatggttcattgtctggattgctcctttcggggataacggtgccctgtacaaatatatgcgaaaaaaattccctgatgaactattcagagccatcagggtggatcccaaaactcatgttggtaaagtatcagaatttcacggaggtaaaattgataaacagttagcgaataaaatttttaaacaatatcaccacgagttaataactgaagtaaaaaacaagtcagatttcaatttttcattaacaggttaagaggtaattaaatgccaacaataaccgctgcacaaattaaaagcacactgcagtctgcaaagcaa

SEQ ID NO: 49 - DNA, 170 bp, Escherichia coli
aggcaggtgtgcgccgcgtactacacattaccgccgttgatgttatcaagcagggcaataatttactcggcgtaataacagagagtaaatctggtcgtcaggctattttggcaaatgtcattattgactgtactggtgatgctgatattgcatggtttgccggagcacca

SEQ ID NO: 50 - DNA, 827 bp, Escherichia coli
ctggcggaggctctgagatcagtagagggtgtggatgttgaaagtggtacgggtaaaaccggagggctggaaatcagcatccgaggaatgccagccagttacacgctgatactgattgatggtgttcgtcagggcggaagcagtgacgtgactcccaacggtttttctgccatgaataccgggttcatgccccctctggccgccattgagcgtattgaggttatcagggggccgatgtccacactgtatggctctgatgcgatgggcggtgtggtgaatatcattaccagaaagaatgcagacaaatggctctcttccgtcaatgcagggctgaatctgcaggaaagcaacaaatggggtaacagcagccagtttaatttctggagcagtggtccccttgtggatgattctgtcagcctgcaggtacgcggtagcacacaacagcgtcagggttcatcggtcacatcactgagcgatacagcaggcacgcgtattccttatcccacggagtcacagaattataatcttggtgcacgtcttgactggaaggcgtcggagcaggatgtgctctggtttgatatggataccacccggcagcgttatgataaccgggatgggcaactggggagtctgacggggggatatgaccggaccctgcgctatgagcgaaacaaaatttcagctggctatgatcatactttcaccttcggaacatggaaatcgtatctgaactggaacgagacagaaaataaaggtcgtgagcttgtacgcagtgtactgaagcgcgacaaatgggggcttgccggtcagccgcgggagcttaagga

SEQ ID NO: 51 - DNA, 258 bp, Escherichia coli
tctgatatagtttatatgggtaataaggctctttatttaatccttatcttttccttatggccagtaggtatagctacggttattggattaactattggtttattacagacagtgactcaacttcaagagcagacacttccttttggtataaagcttataggtgtctcaatatctttgctacttctttctggatggtatggtgaggttttattgtctttttgtcatgaaataatgtttttaattaagagtggggtttga

SEQ ID NO: 52 - DNA, 500 bp, Escherichia coli
ttgcaaaagcaattttgcaacaaactactgcttgatacaaataaggagaatgttatggaaattcaaaacacaaaatcaacccagattttatatacagatatatccacaaaacaaactcaaagttcttccgaaacacaaaaatcacaaaattatcagcagattgcagcgcatattccacttaatgtcggtaaaaatcccgtattaacaaccacattaaatgatgatcaacttttaaagttatcagagcaggttcagcatgattcagaaatcattgctcgccttactgacaaaaagatgaaagatctttcagagatgagtcacacccttactccagagaacactctggatatttccagtctttcttctaatgctgtttctttaattattagtgtagccgttctactttctgctctccgcactgcagaaactaaattgggctctcaattgtcattgattgcgttcgatgctacaaaatcagctgcagagaacattgttcggca

SEQ ID NO: 53 - DNA, 668 bp, Escherichia coli
aagtcaaagcaggggttgcccgaacctttaaagccccaaacctgtatcaatccagtgaaggctatctgctctactcgaaaggcaatggctgtccaaaagatattacatcaggcgggtgctacctgatcggtaataaagatctcgatccggaaatcagcgtcaataaagaaattggactggagttcacctgggaagattaccacgcaagtgtgacctacttccgcaatgattaccagaataagatcgtggccggggataacgttatcgggcaaaccgcttcaggcgcatatatcctcaagtggcagaatggcgggaaagctctggtggacggtatcgaagccagtatgtctttcccactggtgaaagagcgtctgaactggaataccaatgccacatggatgatcacttcggagcaaaaagacaccggtaatcctctgtcggtcatcccgaaatatactatcaataactcgcttaactggaccatcacccaggcgttttctgccagcttcaactggacgttatatggcagacaaaaaccgcgtactcatgcggaaacccgcagtgaagatactggcggtctgtcaggtaaagagctgggcgcttattcactggtggggacgaacttcaattacgatattaataaaaatctgcgtcttaatgtcggcgtc

SEQ ID NO: 54 - DNA, 1689 bp, Escherichia coli
gcgatgtttaaccccgattcggcgcagctggacaatatggcctgggcgcagccggcgattgtcgcgtttgaaatcgcgatggcggcgcactggcacgctgaaggactgaagccagacttcgccattgggcattccgtcggtgaatttgccgctgccgtcgtctgcggacactatacgattgaacaggtcatgccactggtttgtcgacgcggcgcactgatgcagcagtgcgcaagcggcgcaatggtggcggtatttgcagacgaagacacgctgatgccgctggctcgccagtttgagctggatctcgccgccaacaacggtacgcaacatacggtattttccgggccggaagcccgtctcgcggtattttgcaccacgctctcgcagcataacattaactatcgtcgcctgagcgtaaccggcgcggcgcactccgctttactggaaccgatactcgatcggttccaggacgcctgcgcggggctgcacgcggagccggggcaaataccgattatttccacgctcaccgccgacgtcattgatgagtcaacgctcaaccaggcggattactggcgccgacacatgcgccagccggtgcgttttatccagagtattcagatggcgcatcagctcggcgcccgcgtttttctggagatggggcccgatgcccagttggttgcttccgggcagcgcgaataccgcgataacgcatactggatagccagcgcccggcgtaacaaagaggcgagcgatgtcctcaatcaggccctgctccagctttacgctgccggtgtcgccttaccgtggaccgacctactggcgggtgatggacaacgtatcgctgcgccatgttatccgtttgatactgagcgttactggaaagagcgcgtctccccggcctgcgaacctgccgacgcagcgctgtctgccgggctggaggtggcgagtcgcgccgcgacagcgctcgatctcccccgtctggaagcgcttaaacagtgcgccacgcgactgcacgccatctacgtcgatcaactggtacaacgctgtaccggcgatgccattgaaaacggcgtggacgccataaccatcatacgccgtggacgtctgctgccccgctaccagcagctactccagcgcctgctgaataactgcgtggtcgacggcgattaccgctgcaccgacgggcgatacgtccgcgcccaccccattgaacatcaacagcgggaatcactgctgacggaacttgccggttattgtgaaggttttcaggctattcccgacaccatcgcccgtgccggcgatcggttatatgacatgatgagcggcgcggaagaaccggtggcgattatcttcccgcaaagcgcctccgacggcgtggaagtgctgtatcaggaattcagctttggccgctatttcaaccaaatcgccgccggggtattacgcggcattgtccagacgcgtcagccccgccagtcgttgcgtattcttgaagttggcggcggaaccggcggcaccaccgcgtggctgctgccggaactcaacggcgttccggcgctggagtaccacttcaccgatatctcagcgctgttcacccgccgcgcccagcagaaattcgccgactatgattttgtgaagtatagcgagctggatctcgaaaaagaggcgcagtctcagggtttccaggca

SEQ ID NO: 55 - DNA, 1241 bp, Escherichia coli
gccggaaagcctggcctttaaccatccggccagcgccccgtatattcaggaactggcgacaatttgccaacagcttgcacagcgcttacagcgcccggtacgcctgcttgaggtgggaacccgcaccggccgcgccgcagaatcgctgttggcacagctcaacgccggacagattgagtatgtcgggcttgagcagagccaggagatgctactgagcgcccggcagaggctcgcctcctggcctggtgcccgtctgtccccctggaatgcagacacgctggcggcgcacgctcactcgggggacattatctggcttaataacgccctgcatcgtctgctgccggaagatcccgggctccttgcgacattacaacagcttgccgttcccggcgcgctgctctacgtgatggagtttcgccagttaacgccgtccgccctgctcagcacgctcctgttaaccaatgggcagccggaggccttgctgcataacagcgccgactgggcggcattatttagcgcggccgccttcaactgtcagcatagcgatgaggtcgcggggttacaacgcttcctcgtacaatgtcctgacaggcaggtgcgccgcgatccccgtcaacttcaggccgccctcgccgggcgtctgccggggtggatggtgccgcaacggatcgtcttcctcgacgccttaccgctgacggctaacgggaaaattgactaccaggcgctgaagcgtcgtcatacccctaaagcggaaaaccaggccgaagcggatttaccccagggcgacattgaaaaacaggttgccgccctctggcagcaactcttatcgactggcaatgtcaccagagaaaccgacttcttccagcaaggcggcgatagcctgctggcgacccgtctgaccgggcaacttcatcaggcaggttatgaagcgcaattaagcgacctgtttaatcatccccggctggcggattttgccgccacgctgcgtaaaatcgacgtcccggtcgaacaaccattcgtccactctcctgaagaacgctaccagccctttgcgcttaccgacgtgcagcaggcttacctggtggggcgtcagccgggctttaccctgggcggcgtcggctcacatttctttgttgaatttgaaattgccgatctggacctcacccggctggagacggtctggaaccgattaatcgcccgccacgatatgctacgcgccgtcgtgcttgatggacagcaac

SEQ ID NO: 56 - DNA, 607 bp, Escherichia coli
tcacataggattctgccgtttttaacaatgcaggataataagatgaaaaaaatgttattttctgccgctctggcaatgcttattacaggatgtgctcaacaaacgtttactgttggaaacaaaccgacagcagtaacaccaaaggaaaccatcactcatcatttcttcgtttccccaattggacagagaaaactgttgatgcagccaaaatttgttggcggtgcagaaaatgttgttaaaacagaaactcagcaaacattcgtaaatgcattgcccggttttatcacttttggcatctatactccgcgggaaacccgtgtatattgctcacaataggcccatcgatatggggagctcatctgcactgttcattataacttctgggctccctacagttgtttttgcatagtgataagcctctctctgagggaggaaataatcctgttcagcgatgtctaccagtcgggggggctgcattatccaccccgaggcggtggtggcttcacgcggggatgggcagattgatctgatatgcaaccgacgacgaccagcggcaacatcatcacgcagagcttcattttcagatttgggccaccttttgatttct

SEQ ID NO: 57 - DNA, 778 bp, Escherichia coli
aagtgtcgattttattggtgtagggacagggccatttaatctcagcattgctgcgttgtcacatcagatcgaagaactggactgtctcttctttgatgaacatcctcatttttcctggcatccgggtatgctggtaccggattgtcatatgcagaccgtctttctgaaagatctggtcagtgctgttgcacctacaaatccctacagttttgttaactatctggtgaagcacaaaaagttctatcgcttccttacaagcagactacgtacagtatcccgtgaagagttttctgactacctccgctgggctgctgaagatatgaataacctgtatttcagtcataccgttgaaaacattgatttcgataaaaaacgtcgattgtttctggtgcaaaccagccagggacaatattttgcccgcaatatctgccttggtacaggaaaacaaccttatttaccaccctgtgtgaagcatatgacacaatcctgtttccatgccagtgaaagtaatcttcgtcggccggatcttagtggaaaacggataaccgtggttggtggaggacagagtggtgcagacctgttccttaatgcattacgcggggaatggggagaagcggcggaaataaactgggtgtcccggcgtaataattttaacgcactggatgaggctgcttttgctgatgattattttacacctgaatatatttcaggcttctccggactggaggaagatattcgccatcagttactggatgagcagaaaactgacatcggatgg

SEQ ID NO: 58 - DNA, 302 bp, Escherichia coli
ggctggacatcatgggaactggtacgctgaacatcgatgaatcccggcagcttcagttgatcacacagtactataaaagccagggcgacgacgattacgggcttaatctcgggaaaggcttctctgccatcagagggaccagcacgccattcgtcagtaacgggctgaattccgaccgtattcccggcactgacgggcatttgatcagcctgcagtactctgacagcgcttttctgggacaggagctggtcggtcaggtttactaccgcgatgagtcgttgcgattctacccgttcccgacg

SEQ ID NO: 59 - DNA, 2126 bp, Escherichia coli
cttcctgttctgattcttctggcgctatcggggagcttttctaccgctgtagccgctgataaaaaagagactcaaaatttctactatccagaaacactggatttaactcctctgagattacacagccctgaatcaaatccctggggggctgattttgattatgccaccagatttcaacagctggatatggaggctctgaaaaaagatatcaaagatttgctgacaacttcccaggattggtggcctgcggattatggtcattatggtcctttctttattcgtatggcttggcacggtgccggaacatacaggacatatgatggccggggaggcgccagtggtggtcagcaacgttttgaaccgctgaacagctggccggataacgttaatctggataaagcccgtcgattgctgtggccagtcaagaaaaaatacggctccagtatttcctggggagacctgatggtcctgactggtaatgttgcccttgaatccatgggatttaaaacgctgggatttgctggcggaagagaagatgactgggagtcggacctggtatactgggggcctgacaacaagcctcttgcagataaccgggataaaaacgggaaacttcagaaacctcttgccgccacgcagatgggacttatttatgtcaatcctgaaggccccggtggaaaaccagatcctctggcttccgcgaaagatatcagggaagctttttcacgtatggccatggatgatgaggagactgtggccctgatcgcgggagggcatacatttggtaaagcacatggtgcagcgtctcctgaaaaatgtattggcgcagggcctgatggtgcacctgtggaggagcagggactgggatggaaaaataaatgtggtacaggaaacggcaaatataccatcaccagtggcctggaaggagcctggtcgacatcgccaacccagttcacaatgcagtatctgaagaatttatataaatatgaatgggagctgcacaagagtcctgccggtgcttatcagtggaagcctaaaaaagcggcaaatatagttcaggacgcgcatgatccgtctgtcctgcatccgttgatgatgtttacgacggatattgctcttaaagttgatcctgaatataagaaaataaccacccgtttcctgaatgatccaaaagcttttgagcaggcattcgcaagagcatggtttaaactgacccaccgggatatgggaccggcagcccgatatcttggtaatgaagttcctgcagaatcatttatctggcaggatcctcttcctgcggcggattatacaatgattgatggtaaagacattaagtcgctgaaagagcaggttatggatttgggtatccctgcatctgagctgataaagacagcctgggcttcagcttccacatttcgtgtgactgattatcgtgggggaaataatggtgcccgcatcaggttacagcccgaaattaactgggaagttaatgagcctgaaaaactgaagaaagtactggcatccctgacctcattacagcgtgaatttaacaaaaaacagtctgacggaaagaaagtgtcgttggctgatttaattgttctttcgggtaatgctgcaatcgaagatgcggccagaaaagccggggtggaacttgagattccctttactccgggaagaactgacgcctctcaggagcagacggatgttgcctcattcagtgtactggagccgacagcagatggattcagaaattattactcaaaaagcagaagtcatatatcgccggttgaaagcctcattgataaagccagtcagctggatctcaccgttcctgaaatgacggcattactgggtggtctgcgggtaatggatattaatacaaataattcttcgttgggagtgtttaccgatacccctggtgttctggataacaagttttttgttaatctgctggatatgtcaacacgatggagtaaagcagataaagaagatacatacaatggattcgatcgtaaaacgggagcattaaaatggaaagcatcctctgttgatttaatcttcagttcaaatcctgaattacgtgcggtggcagaagtatatgcctcggatgatgcgagaaataagtt

SEQ ID NO: 60 - DNA, 501 bp, Escherichia coli
aattgttttaaaatctgttctttttctgatattgcctgagtgagttttgattctttttcgcttatctcttcttcatatttctgtatgtcatttatttttttttcaatatcatctggatatttttcttgtctcacaacatcgttgtgatttatttttgaaagtttaattacctcttccttttctttttttagctcattaatcctgttcaataataaaaattgtcttttcttaaatatagatttagtcaattcaatatctgcatccttcttttttgactcttgcaccaaatgagatacaatattgaagatagaatttctctcttcgactacctcccataatgcagcctcagccgaaacgttatctttcgatgtttctatataaggtaggtttgcataggcttgtaactcattataaagcttcagaacctcggttctttcttttattaattcagatactaagtactcactaacctttgtatcattaaatgtaatttcagtctca

SEQ ID NO: 61 - DNA, 270 bp, Escherichia coli
gcgcatttgctgatactgttgggcatttttggttacattatgcaccgcacgatgccagacatctcattcccggtgtttttacttaatggcctgattcccttttttatctttagcagtatcagcaatcgttctgtaggcgctattgaagcgaaccaggggttgtttaattatcgaccagtaaaacccatcgatacgatcattgcacgcgcactgcttgagacgctgatttacgttgctgtttatatattgctcatgcttatcgtctggatg

SEQ ID NO: 62 - DNA, 390 bp, Escherichia coli
tcctcttgctactattccccctcaatatcagcattggtttttatggaatccacttgtgcatgctgtagaactaatccgaagggcatggatatctggttatcgtagtcctgatgtaagttgggcgtatctgtcggttgtcaccttattattgctcacttttgctatgagttgttaccgattacggcatcgccaattgattgctagttagcgttaagaaaaatgattattcttgataatgtatcaaaatattatccgactaaatttggacgaaattatgtcctgaggaatgtaaatattgagctaccaagggaccgtaatataggtattctaggtatcaatggagcaggaaaatctactttgttacgtttgttaggagggatggatacgcct

SEQ ID NO: 63 - DNA, 659 bp, Escherichia coli
cagttcgctcgtaaagcagaaaaatgcgacagaagatgttgttttaataggcaaaatgattttagatgaagttagaagttacagaactatacataatgatcgaaatatcgtaagtaactcaggaaactggaaaacatcttttttatgtaatcttgctagactactatatagcatatttaatggtagtaactatttttgttcccgagagggtgaaaataattcatcccccagttctactctacttactatacatcagcctgaaaagcaggaactattacaacaaaagagtatcaaacatttaccaacaagtaataacatcgacggatacattaaaataagaaaaacaagaggcgctgaagatcaaacaacaactatcactcaaagtttgataattaatgagttgttaaatggagttgatagaaataccatcccttttcagaaaataagtgagctcaatgatatcatacattcatatgaaaatatgcaaattaaaaatagtcgaaaaggtatagaaatacttgttaagcagggagagctgttatcatcattaataaatgataataaaggaaataaacaattatcagacaatgcatctaaaataataaacttattgggtatagagtatcagtcacataaagtagacatagagccatttatac

SEQ ID NO: 64 - DNA, 501 bp, Escherichia coli
gaacaattcaaacagttcagtattgaaaaacaggctgcgattaactcgctattacagttgcgcggaatgttagaaatgctgggagagatggggataaacatcagcgacgatttacaaaaagtcacttctgcaattaatgccatcgaatctgatgtcctgcgtattgctctgttgggggcgttctccgatggcaaaaccagcgttatcgccgcatggctgggtaaagtaatggatgatatgaatatttccatggatgagtcctccgatcggttgagtatttacaaaccggaaggtctgccagatcagtgtgaaattgttgatacgcccgggctgtttggtgataaagagcgcgaggtggacgggagactggtgatgtatgaagacctgaccagacgctatatatctgaagcacacctgattttttacgtggttgatgccacgaacccgctcaaggagagccacagcgacatcgtaaaatgggtattgcgcgatttgaataaa

SEQ ID NO: 65 - DNA, 424 bp, Escherichia coli
caaatacagtccgcgtacgaatgaaagatgcttatcaacgtgatggtaaatatccagattttgtggacccattaagccttactgcaaatacaattaaaactgatacaagcggaatacctgcagcacagttagttcagcttgggaaaattacaccagacgaagtgcgtaataacatttctggcgactttatcgctattggcggtgctttaacttcgaatggtgctcaagttaaaaaaggttttgctatcgaacttaatggattaagccaagagcagtgccgttctattcttgggcaagttgggaataactgggaatatgttgctattggtacttctgcgtctggttcatatgccatgacagcaactggtgtagatatgtctgtggccgcctctacaactgttttacgctctttaggtaacaatgg

SEQ ID NO: 66 - DNA, 275 bp, Escherichia coli
ttacggcgttactatcctctctatgtgcatacggagctccccagtctattacagaactatgttcggaatatcgcaacacacaaatatatacgataaatgacaagatactatcatatacggaatcgatggcaggcaaaagagaaatggttatcattacatttaagagcggcgcaacatttcaggtcgaagtcccgggcagtcaacatatagactcccaaaaaaaagccattgaaaggatgaaggacacattaagaatcacatatctgaccgagacc

SEQ ID NO: 67 - DNA, 500 bp, Escherichia coli
ttggcagttacaggaatgcattgtgataatgcgtatggaaatacaatacatattatagaacaagataattttaatattatcaaggttgtggatataaatatcaatacaacttcacatactcacattctccattcaatgagtgtttgcctcaattcgtttggtgattttttttcaaataacacatatgatgcggttatggttttaggcgatagatatgaaatattttcagtcgctatcgcagcatcaatgcataatattccattaattcatattcatggtggtgaaaagacattagctaattatgatgagtttattaggcattcaattactaaaatgagtaaactccatcttacttctacagaagagtataaaaaacgagtaattcaactaggtgaaaagcctggtagtgtgtttaatattggttctcttggtgcagaaaatgctctttcattgcatttaccaaataagcaggagttggaactaaaatatggttcactgtt

SEQ ID NO: 68 - DNA, 537 bp, Escherichia coli
gcttactgattctgggatggattaacagaacacaactggcttgtccataagcaaaatgaaggcaaaaaaatatgaaaatcaaatatacaatgaaaatggccgccgttgccagcgtcatggtcgccggctagctatcgcggatgcaaatgggctcaacactgtgaacgccggggatggcaagaatctgggcaccgcaaccgcgacgatcaccactctgcagagctgctctgtcgacctgaatctcgttaccccgaacgcgacagtgaacagagcaggaatgctagcaaaccgcgaaatcactaaattttcggtggggagtaaggattgccctagcgacacctatgctgtatggtttaaagagatcgatggcgaaggacagggggtcgcgcagggcactacggtgaccaacaagttttaccttaaaatgacatcggccgacgggaccgcgagcgtaggggacatcaacataggaaccaaatcaggcaaaggcctgagtggtcaactggtagggggaaaattcgacggaaaaataacggt

SEQ ID NO: 69 - DNA, 1422 bp, Escherichia coli
cttgcggaggcttgtctgagcggtttccgcgattctcttctgtaaattgtcgctgacaaaaaagattaaacataccttatacaagacttttttttcatatgcctgacggagttcacacttgtaagttttcaactacgttgtagactttacatcgccaagggtgctcggcataagccgaagatatcggtagagttaatattgagcagatcccccggtgaaggatttaaccgtgttatctcgttggagatattcatggcgtattttggatgataacgaggcgcaaaaaatgaaaaagacagctatcgcgattgcagtggcactggctggtttcgctaccgtagcgcaggccgctccgaaagataacacctggtacactggtgctaaactgggctggtcccagtaccatgatactggtttcatcaacaacaatggcccgacccatgaaaaccaactgggcgctggtgcttttggtggttaccaggttaacccgtatgttggctttgaaatgggttacgactggttaggtcgtatgccgtacaaaggcagcgttgaaaacggtgcatacaaagctcagggcgttcaactgaccgctaaactgggttacccaatcactgacgacctggacatctacactcgtctgggtggcatggtatggcgtgcagacactaaatccaacgtttatggtaaaaaccacgacaccggcgtttctccggtcttcgctggcggtgttgagtacgcgatcactcctgaaatcgctacccgtctggaataccagtggacgaacaacatcggtgacgcacacaccatcggcactcgtccggacaacggcatgctgagcctgggtgtttcctaccgtttcggtcagggcgaggcagctccagtagttgctccggctccagctccggcaccggaagtacagaccaagcacttcactctgaagtctgacgttctgttcaacttcaacaaagcaaccctgaaaccggaaggtcaggctgctctggatcagctgtacagccagctgagcaacttggatccgaaagacggttccgtagttgttctgggttacaccgaccgcatcggttctgacgcttacaaccagggtctgtccgagcgccgtgctcagtctgttgttgattacctgatctccaaaggtatcccggcagacaagatctccgcacgtggtatgggcgaatccaacccggttactggcaacacctgtgacaacgtgaaacagcgtgctgcactgatcgactgcctggctccggatcgtcgcgtagagatcgaagttaaaggtatcaaagacgttgtaactcagccgcaggcttaagttctcgtctggtagaaaaacgctgctgcgggtttttttttgcctttagtaaattgaactgactttcgtcagttattccttacccagcaatgcct

SEQ ID NO: 70 - DNA, 559 bp, Escherichia coli
atctagccgaagaaggaggccgaaaagtcagtcaactcgactggaaattcaataacgctgcaattattaaaggtgcaattaattgggatttgatgccccagatatctatcggggctgctggctggacaactctcggcagccgaggtggcaatatggtcgatcaggactggatggattccagtaaccccggaacctggacggatgaaagtagacaccctgatacacaactcaattatgccaacgaatttgatctgaatatcaaaggctggctcctcaacgaacccaattaccgcctgggactcatggccggatatcaggaaagccgttatagctttacagccagaggtggttcctatatctacagttctgaggagggattcagagatgatatcggctccttcccgaatggagaaagagcaatcggctacaaacaacgttttaaaatgccctacattggcttgactggaagttatcgttatgaagattttgaactcggtggcacatttaaatacagcggctgggtggaatcatctgataacgatgaacactatgacccggg

SEQ ID NO: 71 - DNA, 360 bp, Escherichia coli
atgaggaacataatggcaggttttttaatattcctgtcttctgctgcttatgctgatatcaatctgtatggtcctggtggcccgcatacagccttgcttgatgcagccaaactttatgccgaaaaaacaggtattatagtgaacgttcattacggcccacagaacaaatggaatgaagatgccaaaaaaaatgcagatatcttgtttggcgcatcagaacaatctgctctggctatcattcgggaccataaagacagcttcagtgaaaaagatattcagcctctttatctgcgaaaaagtattttactggtaaagaaaggtaatcctaaaaatatccggagtattgacgacctgaccaga

SEQ ID NO: 72 - DNA, 721 bp, Escherichia coli
atggcagtggtgtcttttggtgtaaataatgctgctccaactattccacaggggcagggtaaagtaacttttaacggaactgttgttgatgctccatgcagcatttctcagaaatcagctgatcagtctattgattttggacagctttcaaaaagcttccttgaggcaggaggtgtatccaaaccaatggacttagatattgaattggttaattgtgatattactgcctttaaaggtggtaatggcgccaaaaaagggactgttaagctggcttttactggcccgatagttaatggacattctgatgagctagatacaaatggtggtacgggcacagctatcgtagttcagggggcaggtaaaaacgttgtcttcgatggctccgaaggtgatgctaataccctgaaagatggtgaaaacgtgctgcattatactgctgttgttaagaagtcgtcagccgttggtgccgctgttactgaaggtgccttctcagcagttgcgaatttcaacctgacttatcagtaatactgataatccggtcggtaaacagcggaaatattccgctgtttatttctcagggtatttatcatgagactgcgattctctgttccacttttcttttttggctgtgtgtttgttcatggtgtttttgccggtccgtttcctccgcccggcatgtcccttcctgaatactggggagaagagcacgtatggtgggacg

SEQ ID NO: 73 - DNA, 318 bp, Escherichia coli
gacggctgtactgcagggtgtggcggttggattgtcagcctcaaggtctaaatatctggggcgtgataacgattctgcttacctgcgtatatccgtgccgctggggacggggacagcgagctacagtggcagtatgagtaatgaccgttatgtgaatatggccggctacactgacacgttcaatgacggtctggacagctacagcctgaacgccggccttaacagtggcggtggactgacatcgcaacgtcagattaatgcctattacagtcatcgtagtccgctggcaaatttgtccgcgaatattgcatccctgca

SEQ ID NO: 74 - DNA, 336 bp, Escherichia coli
gcaacagcaacgctggttgcatcatattcgtaatagtatcaactaaaatacgttaattttatatctcgtaaaataaaatgttttctgtaccgctctccggagggggaatgattcgtttatcattatttatatcgttgcttctgacatcggtcgctgtactggctgatgtgcagattaacatcagggggaatgtttatatccccccatgcaccattaataacgggcagaatattgttgttgattttgggaatattaatcctgagcacgtggacaactcacgtggtgaagtcacaaaaaccataagcatatcctgtccgtataagagtggctctctct

SEQ ID NO: 75 - DNA, 461 bp, Escherichia coli
tcgtgctcaggtccggaatttgcgagtggagtgtattttcaggagtatctggcctggatggttgttcctaaacatgtctatactaatgaggggtttaatatatttcttgatgttcagagcaaatatggttggtctatggagaatgaaaatgacaaagatttttacttctttgttaatggttatgaatgggatacatggacaaataatggtgcccgtatatgtttctatcctggaaatatgaagcagttgaacaataaatttaatgatttagtattcagggttcttttgccagtagatctccccaagggacattataattttcctgtgagatatatacgtggaatacagcaccattactatgatctctggcaggatcattataaaatgccttacgatcagattaagcagctacctgccactaatacattgatgttatcattcgataatgttgggggatgcca

SEQ ID NO: 76 - DNA, 190 bp, Escherichia coli
gggatgagcgggcctttgatgcaggtaatttgtgtcagaaaccaggagaaacaactcgtctgactgagaaatttgacgatattatttttaaagtcgccttacctgcagatcttcctttaggggattattctgttacaattccatacacttccggcatacagcgtcatttcgcgagttacttgggggcccg

SEQ ID NO: 77 - DNA, 268 bp, Escherichia coli
taagctatgtggcctgcaatggatttacctggactcatggtctttactggtctgagtattttgcatggctggttgttcctaaacatgtttcctataatggatataatatatatcttgaacttcagtccagaggaagtttttcacttgatgcagaagataatgataattactatcttaccaagggatttgcatgggatgaagcaaacacatctggacagacatgtttcaatatcggagaaaaaagaagtctggcatggtcatttggtgg

SEQ ID NO: 78 - DNA, 922 bp, Escherichia coli
tcgccaccaatcacagccgaaccgccgattggcgtaaagcggaaaactgacgtcaccagatggtttaagccaaaaggaatcgtcacgcgttcggcaactgcatagaagaaataaccaacaggaccggaagttgaaatccagtgtccaatgagcatgaaaagattgaaaaacggcggccagataaaaggaatgatcagaccaaatccactcatcacaatcagtgtaatgataggcaccagacgtgggccgctataagaacctaacgattcaggaatgcgtaaattaacgatctttttatacatgctggcgactaataacccagcaacaattccccccaacacgctggtattgtaggactggatccccagaatgatggtttgcccatgtgtcgacatttggtcagcaacgaccaataagtcgtgctgtttaagataaaagttcgttcccagatgcatcgccataaaaccaattaagccagaaaaagcaccataggctttatcctctttatcttttaataatcctaagggaatcgctatcgcaaacaatacaggtaaattaacaaaggcaaacaaaccaagactaacaatgaaatcaagtatggttttaattattggaatagccagaaatggaattaactttgccatatcatcactggctaaaccacttcccagccctagcatcatgccacatacacttagcagagcaatgggatacataaatgccttccccaggctctgaaaaaaactccaggctttcttttgtttcatgtgggttatctcatataaatgttatatataattagtccattaatactttggtacgaatagagagatatagtttttcttctaaaattaattcatatttaaaagtggcatacagataccgttcaatttcatgaattgcgcgctgtaacaggatgtcc

SEQ ID NO: 79 - DNA, 501 bp, Escherichia coli
ggtgatcgattattccgctgagcgtattcagtctttaaaagacaaatacagcctgccggatgagtttatcttgtcgctggcgatgatcgagccgcggaaaaatattgaagcgcttattcacgcctacagcttgctgcctgccgagctgcagcagcgctatccgatggtgctggcgtataaagtgcagccagaacaactggagcggatcctgcgtctggcggaaagctatggtttgtcacgcagccagcttatctttactgggttcctgaccgacgacgatctgattgccctgtacaacctgtgcaaactgtttgtgttcccgtcgctgcatgaaggtttcggcctgccgccgctggaagcgatgcgctgcggggcggcgaccttaggttcaaacattaccagcctgccggaagtcattggctgggaagatgccatgttcaatccgcatgatgtgcaggacattcgccgggtcatggagaaggcgctgaccgatgaggcgtt

SEQ ID NO: 80 - DNA, 500 bp, Escherichia coli
tctgcacgtttaaaattattgcctgggttaaagtcaactgagtatgtgtattcagatcttcatgctttacttgatactaatggtgggagttccttaggtccgaacattggtagtgatggttctaacctaacaataacatgtttatcgatgagcagagtttttcttactgaaaaacttgttaattctatatatcagcatataccttattttaaaggtgatattctgattgttgataatggcagcacagtagaagaactttcaattttacaagatttaagtgataggatcccgttaaatattagagttgtcgagcttggtaataattttggcgtaagtggtggaagaaacaaaactttagagcatataaaaacagaatgggcaatgtttctcgataatgatatttatttcataaataatccacttccgagattgcaaaatgatatttcaagacttggttgtcattttatcaatatgccattgcttgattctgacggagaaac

SEQ ID NO: 81 - DNA, 406 bp, Escherichia coli
tagagaaattatcaagttagttccattagtatcaattgatctgctaattgaaaacgagaatggtgaatatttatttggtcttaggaataatcgaccggccaaaaattatttttttgttccaggtggtaggattcgcaaaaatgaatctattaaaaatgcttttaaaagaatatcatctatggaattaggtaaagagtatggtatttcaggaagtgtttttaatggtgtatgggaacatttctatgatgatggttttttttctgaaggcgaggcaacacattatatagtgctttgttacacactgaaagttcttaaaagtgaattgaatctcccagatgatcaacatcgtgaatacctttggctaactaaacaccaaataaatgctaaacaagatgttcataactat

SEQ ID NO: 82 - DNA, 292 bp, Escherichia coli
gtgtccatttatacggacatccatgtgatatggaacaaattgtagaactggccaaaagtagaaatttgtttgtaattgaagattgcgctgaagcctttggttctaaatataaaggtaaatatgtgggaacatttggagatatttctacttttagcttttttggaaataaaactattactacaggtgaaggtggaatggttgtcacgaatgacaaaacactttatgaccgttgtttacattttaaaggccaaggattagctgtacataggcaatattggcatgacgttatagg

SEQ ID NO: 83 - DNA, 259 bp, Escherichia coli
cggacatccatgtgatatggaacaaattgtagaactggccaaaagtagaaatttgtttgtaattgaagattgcgctgaagcctttggttctaaatataaaggtaaatatgtgggaacatttggagatatttctacttttagcttttttggaaataaaactattactacaggtgaaggtggaatggttgtcacgaatgacaaaacactttatgaccgttgtttacattttaaaggccaaggattagctgtacataggcaa

SEQ ID NO: 84 - DNA, 786 bp, Escherichia coli
atccatcaggaggggactggataggttattttctccattatgactgcatggttaatgagcagtgtaataatggttttataatgtttgaacctggatatgaattaattgtttccttatttggatatttgggatttcagacaattattatttttatagccgctgtaaatgtaattctaatattaaattttgcaaagcattttgaaaacggaagttttgttattgttgcgataatgtgcatgttcctttggagtgtttatgttgaggcgattagacaggctctggccttatctatagttatatttgggattcattctctttttttgggtagaaaaaggaaatttataacattagtattatttgcgtcaactttccatataactgctttgatttgttttcttctaatgactcctctattttcaaagaaattaagcaagataataagttatagcctattaattttcagtagcttctttttcgctttttctgaaaccatattaagtgcactccttgcaattttgccagaaggatccattgccagtgaaaaattaagtttttacttagcaaccgagcaatacaggccacagttatctattgggagtggcactattcttgacattatacttatttttctgatatgtgtaagttttaaacgaataaagaaatatatgctcgctaattataatgctgcaaatgagatattgcttattggttgctgtctttatatttctttcggtatttttatcgggaaaatgatgccagttatgactcgcattggttggtatggtt

SEQ ID NO: 85 - DNA, 521 bp, Escherichia coli
ctaccgtagcgggcgatggtagctggacaaccaccgtacccgccgccgatctcagcgtgttacgcgacggcgacgccaccgtgcaggccagcgtcagcactattaacggcaacacggcttcggcaacccacgcctacagcgtcgatgccacggccccgacgcttgccattaacaccatcgccaccgacgatattctgaacgctgccgaggcgggcaatccgttaaccatcagcggtagcagcaccgccgaagcggggcagacggtaaccgtcacgcttaatggtgtgacttacagcggctccgtccaggcggacggcagctggagcgtcagcttaccgacggcggatctcagcaatctgaccgccagccagtacaccgttagtgcctcggtaagcgataaagcgggtaacccggcgtccgctaaccacgggctggcggtggatctcaccgtgccggtgctgaccatcaacaccgtctccggcgatgacattattaacgccgccgaacacggacaggcgctg

SEQ ID NO: 86 - DNA, 408 bp, Escherichia coli
ctccggagaactgggtgcatcttacccgcggagatatgaaactgcatatgcaggcgaggtataaggccacacattatcccgtcgccgggggaaaggcaaatggacaggtatggttttctctgacctatctgtaactggcagatataatgccatttaattaaggctgttaataacatgatgaagcacatgcgtatatgggccgttctggcatcatttttagtctttttttatattccgcagagctatgccggggttgctctgggtgccacccgtgagatttaccctgaagggcaaaaacaggtacaactggcggtaacaaataatgatgataaaagtagttaccttattcagtcatggattgaaaatgctgaaggaaaaaaggatgccaggtttgtaattactcctccg

SEQ ID NO: 87 - DNA, 500 bp, Escherichia coli
ccctgaccttgggtgttgcgacaaatgcgtctgctgtcaccacggttaatggtggtacagttcattttaagggggaagttgttgatgctgcatgtgctgtaaacactaattcagcaaatcaaacgttttctgggcaagttcgttcagctaagttggcgaatgatggagagaagagttcccctgttggatttagtattgaacttaatgactgtagttctgcaactgccgggcatgcatcaattatctttgcaggaaatgttattgctacacacaatgatgtgctgtctctacagaatagtgctgcaggtagtgcaacaaatgtaggtattcagatattggatcatacaggtactgcagttcaatttgacggagtgactgcatctacacaatttacattaacagatggcaccaataaaattcctttccaggcagtttattatgcaacaggtaagtcaacgcctggtattgccaacgccgacgccacctttaaagttcagtac

SEQ ID NO: 88 - DNA, 214 bp, Escherichia coli
aagaaatcaatattatttatttttctttctgtattgtctttttcacctttcgctcaggatgctaaaccagtagagtcttcaaaagaaaaaatcacactagaatcaaaaaaatgtaacattgcaaaaaaaagtaataaaagtggtcctgaaagcatgaatagtagcaattactgctgtgaattgtgttgtaatcctgcttgtaccgggtgctatt

SEQ ID NO: 89 - DNA, 163 bp, Escherichia coli
tcccctcttttagtcagtcaactgaatcacttgactcttcaaaagagaaaattacattagagactaaaaagtgtgatgttgtaaaaaacaacagtgaaaaaaaatcagaaaatatgaacaacacattttactgctgtgaactttgttgtaatcctgcctgtgc

SEQ ID NO: 90 - DNA, 368 bp, Escherichia coli
gcaataaggttgaggtgattttatgaaaaagaatatcgcatttcttcttgcatctatgttcgttttttctattgctacaaatgcctatgcatctacacaatcaaataaaaaagatctgtgtgaacattatagacaaatagccaaggaaagttgtaaaaaaggttttttaggggttagagatggtactgctggagcatgctttggcgcccaaataatggttgcagcaaaaggatgctaatatatttatcaatagcattcagcaccatatacacaaaaataatttttcataaaaagaactctataaaataaatattttttgtgacaatgtcctaacgcaagacggacattgtccatttctcactgcaggc

SEQ ID NO: 91 - DNA, 583 bp, Escherichia coli
acactggatgatctcagtgggcgttcttatgtaatgactgctgaagatgttgatcttacattgaactggggaaggttgagtagtgtcctgcctgattatcatggacaagactctgttcgtgtaggaagaatttcttttggaagcattaatgcaattctgggaagcgtggcattaatactgaattgtcatcatcatgcatcgcgagttgccagaatggcatctgatgagtttccttctatgtgtccggcagatggaagagtccgtgggattacgcacaataaaatattgtgggattcatccactctgggggcaattctgatgcgcagaactattagcagttgagggggtaaaatgaaaaaaacattattaatagctgcatcgctttcatttttttcagcaagtgcgctggcgacgcctgattgtgtaactggaaaggtggagtatacaaaatataatgatgacgatacctttacagttaaagtgggtgataaagaattatttaccaacagatggaatcttcagtctcttcttctcagtgcgcaaattacggggatgactgtaaccattaaaactaatgcctgtc

SEQ ID NO: 92 - DNA, 1612 bp, Escherichia coli
agtcctcgatggcggtccattatctgcattatgcgttgttagctcagccggacagagcaattgccttctgagcaatcggtcactggttcgaatccagtacaacgcgccatatttatttaccaggctcgcttttgcgggccttttttatatctgcgccgggtctggtgctgattacttcagccaaaaggaacacctgtatatgaagtgtatattatttaaatgggtactgtgcctgttactgggtttttcttcggtatcctattcccgggagtttacgatagacttttcgacccaacaaagttatgtctcttcgttaaatagtatacggacagagatatcgacccctcttgaacatatatctcaggggaccacatcggtgtctgttattaaccacaccccaccgggcagttattttgctgtggatatacgagggcttgatgtctatcaggcgcgttttgaccatcttcgtctgattattgagcaaaataatttatatgtggccgggttcgttaatacggcaacaaatactttctaccgtttttcagattttacacatatatcagtgcccggtgtgacaacggtttccatgacaacggacagcagttataccactctgcaacgtgtcgcagcgctggaacgttccggaatgcaaatcagtcgtcactcactggtttcatcatatctggcgttaatggagttcagtggtaatacaatgaccagagatgcatccagagcagttctgcgttttgtcactgtcacagcagaagccttacgcttcaggcagatacagagagaatttcgtcaggcactgtctgaaactgctcctgtgtatacgatgacgccgggagacgtggacctcactctgaactgggggcgaatcagcaatgtgcttccggagtatcggggagaggatggtgtcagagtggggagaatatcctttaataatatatcagcgatactggggactgtggccgttatactgaattgccatcatcagggggcgcgttctgttcgcgccgtgaatgaagagagtcaaccagaatgtcagataactggcgacaggcctgttataaaaataaacaatacattatgggaaagtaatacagctgcagcgtttctgaacagaaagtcacagtttttatatacaacgggtaaataaaggagttaagcatgaagaagatgtttatggcggttttatttgcattagcttctgttaatgcaatggcggcggattgtgctaaaggtaaaattgagttttccaagtataatgaggatgacacatttacagtgaaggttgacgggaaagaatactggaccagtcgctggaatctgcaaccgttactgcaaagtgctcagttgacaggaatgactgtcacaatcaaatccagtacctgtgaatcaggctccggatttgctgaagtgcagtttaataatgactgaggcataacctgattcgtggtatgtgggtaacaagtgtaatctgtgtcacaattcagtcagttgacagttgcctgtcagactgagcatttgttaaaaaaatttcgcatggtgaatccccctgtgtggaggggcgactggtgaaaaatccttgcttgtgattcattatcgacac

SEQ ID NO: 93 - DNA, 502 bp, Escherichia coli
gcgaaggaatttaccttagacttctcgactgcaaagacgtatgtagattcgctgaatgtcattcgctctgcaataggtactccattacagactatttcatcaggaggtacgtctttactgatgattgatagtggctcaggggataatttgtttgcagttgatgtcagagggatagatccagaggaagggcggtttaataatctacggcttattgttgaacgaaataatttatatgtgacaggatttgttaacaggacaaataatgttttttatcgctttgctgatttttcacatgttacctttccaggtacaacagcggttacattgtctggtgacagtagctataccacgttacagcgtgttgcagggatcagtcgtacggggatgcagataaatcgccattcgttgactacttcttatctggatttaatgtcgcatagtggaacctcactgacgcagtctgtggcaagagcgatgttacggtttgttactgtgacagctg

SEQ ID NO: 94 - DNA, 482 bp, Escherichia coli
cttgaacatatatctcaggggaccacatcggtgtctgttattaaccacaccccaccgggcagttattttgctgtggatatacgagggcttgatgtctatcaggcgcgttttgaccatcttcgtctgattattgagcaaaataatttatatgtggccgggttcgttaatacggcaacaaatactttctaccgtttttcagattttacacatatatcagtgcccggtgtgacaacggtttccatgacaacggacagcagttataccactctgcaacgtgtcgcagcgctggaacgttccggaatgcaaatcagtcgtcactcactggtttcatcatatctggcgttaatggagttcagtggtaatacaatgaccagagatgcatccagagcagttctgcgttttgtcactgtcacagcagaagccttacgcttcaggcagatacagagagaatttcgtcaggcactgtctgaaactgctcctgt

SEQ ID NO: 95 - DNA, 151 bp, Escherichia coli
ggtggagtatacaaaatataatgatgacgatacctttacagttaaagtgggtgataaagaattatttaccaacagatggaatcttcagtctcttcttctcagtgcgcaaattacggggatgactgtaaccattaaaactaatgcctgtcat

SEQ ID NO: 96 - DNA, 211 bp, Escherichia coli
ttctgttaatgcaatggcggcggattgtgctaaaggtaaaattgagttttccaagtataatgaggatgacacatttacagtgaaggttgacgggaaagaatactggaccagtcgctggaatctgcaaccgttactgcaaagtgctcagttgacaggaatgactgtcacaatcaaatccagtacctgtgaatcaggctccggatttgctgaa

SEQ ID NO: 97 - DNA, 226 bp, Escherichia coli
gaagaagatgtttatagcggttttatttgcattggtttctgttaatgcaatggcggcggattgtgctaaaggtaaaattgagttttccaagtataatgaggataatacctttactgtgaaggtgtcaggaagagaatactggacgaacagatggaatttgcagccattgttacaaagtgctcagctgacagggatgactgtaacaatcatatctaatacctgcagt

SEQ ID NO: 98 - DNA, 442 bp, Escherichia coli
attggtgccggtgttactgctgctcttcatcggaaaaaccaaccggcagaacaaacaatcactacacgtacggtagtcgataatcagcctacgaataacgcatctgcgcagggcaatactgacacaagtgggccagaagagtccccggcgagcagacgtaattcgaatgccagcctcgcatcgaacgggtctgacacctccagcacgggcacggtagagaatccgtatgctgacgttggaatgcccagaaatgattcactggctcgcatttcagaggaacctatttatgatgaggtcgctgcagatcctaattatagcgtcattcaacatttttcagggaacagcccagttaccggaaggttagtgggaaccccagggcaaggtatccaaagtacttatgcgcttctggcaagcagcggcggattgcgtttaggtatgggag

SEQ ID NO: 99 - DNA, 1521 bp, Escherichia coli
atgcctattggtaatcttggtcataatcccaatgtgaataattcaattcctcctgcacctccattaccttcacaaaccgacggtgcaggggggcgtggtcagctcattaactctacggggccgttgggatctcgtgcgctatttacgcctgtaaggaattctatggctgattctggcgacaatcgtgccagtgatgttcctggacttcctgtaaatccgatgcgcctggcggcgtctgagataacactgaatgatggatttgaagttcttcatgatcatggtccgctcgatactcttaacaggcagattggctcttcggtatttcgagttgaaactcaggaagatggtaaacatattgctgtcggtcagaggaatggtgttgagacctctgttgttttaagtgatcaagagtacgctcgcttgcagtccattgatcctgaaggtaaagacaaatttgtatttactggaggccgtggtggtgctgggcatgctatggtcaccgttgcttcagatatcacggaagcccgccaaaggatactggagctgttagagcccaaagggaccggggagtccaaaggtgctggggagtcaaaaggcgttggggagttgagggagtcaaatagcggtgcggaaaacaccacagaaactcagacctcaacctcaacttccagccttcgttcagatcctaaactttggttggcgttggggactgttgctacaggtctgatagggttggcggcgacgggtattgtacaggcgcttgcattgacgccggagccggatagcccaaccacgaccgaccctgatgcagctgcaagtgaaactgaaactgcgacaagagatcagttaacgaaagaagcgttccagaacccagataatcaaaaagttaatatcgatgagctcggaaatgcgattccgtcaggggtattgaaagatgatgttgttgcgaatatagaagagcaggctaaagcagcaggcgaagaggccaaacagcaagccattgaaaataatgctcaggcgcaaaaaaaatatgatgaacaacaagctaaacgccaggaggagctgaaagtttcatcgggggctggctacggtcttagtggcgcattgattcttggtgggggaattggtgttgccgtcaccgctgcgcttcatcgaaaaaatcagccggtagaacaaacaacaacaacaactactacaactacaactacaagcgcacgtacggtagagaataagcctgcaaataatacacctgcacagggcaatgtagatacccctgggtcagaagataccatggagagcagacgtagctcgatggctagcaccttgtcgactttctttgacacttccagcatagggaccgtgcagaatccgtatgctgatgttaaaacatcgctgcatgattcgcaggtgccgacttctaattctaatacgtctgttcagaatatggggaatacagattctgttgtatatagcaccattcaacatcctccccgggatactactgataacggcgcacggtta

SEQ ID NO: 100 - DNA, 446 bp, Escherichia coli
attggtgctggtgtaacgactgcgctccatagacgaaatcagccggcagaacagacaactactacaacaacacatacggtagtgcagcagcagaccggagggaataccccagcacaaggtggcactgatgccacaagagcagaagatgcttctctgaatagacgtgattcgcaggggagtgttgcatcgacacactggtcagattcctctagcgaagtggttaatccatatgctgaagttggggagcctcggaatagtctatcgactcgtcagcaagaagagcatatttacgatgaggtcgctgcagatcctgtttatagcgtcattcagaatttttcacggaatgctccagttaccggaaggttaatgggaagcccagggcaaggtatccaaagtacttatgcgcttctggcaaacagcgctggattgcgtttaggtatgggagg

SEQ ID NO: 101 - DNA, 288 bp, Escherichia coli
ggtgtggtgcgatgagcacagcaatcaagaagcgtaaccttgaggtgaagactcagatgagtgagaccatctggcttgaacccgccagcgaacgcacggtatttctgcagatcaaaaacacgtctgataaagacatgagtgggctgcagggcaaaattgctgatgctgtgaaagcaaaaggatatcaggtggtgacttctccggataaagcctactactggattcaggcgaatgtgctgaaggccgataagatggatctgcgggagtctcagggatggctgaaccgtg

SEQ ID NO: 102 - DNA, 640 bp, Escherichia coli
ggtggtgcactggagtggagctttaacagcagtaccggagctggtgcgctgacacagggaaccaccacatatgccatgcacgggcagcagggaaatgacctgaatgctggtaagaacctgatatttcaggggcagaatggtcagattaaccttaaggattcggtttctcagggggcgggttccctgacgttccgtgataattacacagtaacaacctctaacggaagtacctggaccggtgccggtattgttgtggacaacggggtgtccgtaaactggcaggttaatggtgttaagggcgataacctgcataaaattggtgaaggtacgctgacggtacagggtacaggtattaatgaaggtggcctgaaggtcggggacggaaaggttgtactgaaccagcaggcggacaataaaggacaggtgcaggcgttcagcagtgttaatattgccagtggccggccgaccgtggtactgactgatgagcggcaggtaaatccggataccgtctcatggggatatcgtgggggcacactggatgttaatggtaacagtctgacgtttcatcagttgaaggcggcagattatggtgccgtgctggcgaataacgttgataaacgggccactatcacgctggact

SEQ ID NO: 103 - DNA, 250 bp, Escherichia coli
gcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatca

SEQ ID NO: 104 - DNA, 501 bp, Escherichia coli
ctactgttcccgagtagtgtgttggcgactcaaatatggggaaaatggtcgctcagtggcgtactcagtgcaacccgcggctcttacatcggtgcgttggcatctgctttgtatattccctctgcgggcgagggcagtgctcgcgtgcccggacgtgatgagttctggtatgaggaagaactgcggcagaaagcactagcaggcagtaccgccaccacccgggtacgttttttctggggaactgacattcacggcaagcctcaggtgtatggtgttcatacgggtgaaggtacgccgtatgaaaacgtccgcgtggcgaacatgcagtggaacgagcagacgcagcgttatgaatttacccccgctcacgatgtcgatggccccctgattacctggacgccggaaaatccggaacatgggaatgttccgggccataccggtaacgacaggccgccgctggatcagcccaccattctggtgacgccgattccggacggcacc

SEQ ID NO: 105 - DNA, 22 bp, Artificial sequence
  Artificial Sequence = Primer
gcgatcatggccgcgaccagca

SEQ ID NO: 106 - DNA, 22 bp, Artificial sequence
  Artificial Sequence = Primer
caactcacccagtagccccagt

SEQ ID NO: 107 - DNA, 22 bp, Artificial sequence
  Artificial Sequence = Primer
gaaagtaaatggaatataaatg

SEQ ID NO: 108 - DNA, 23 bp, Artificial sequence
  Artificial Sequence = Primer
tttgtgttgccgccgctggtgaa

SEQ ID NO: 109 - DNA, 22 bp, Artificial sequence
  Artificial Sequence = Primer
gaaagtaaatggaatataaatg

SEQ ID NO: 110 - DNA, 23 bp, Artificial sequence
  Artificial Sequence = Primer
tttgtgtcggtgcagcagggaaa

SEQ ID NO: 111 - DNA, 21 bp, Artificial sequence
  Artificial Sequence = Primer
ggtgcaatggctctgaccaca

SEQ ID NO: 112 - DNA, 21 bp, Artificial sequence
  Artificial Sequence = Primer
gtcattacaagagatactact

SEQ ID NO: 113 - DNA, 21 bp, Artificial sequence
  Artificial Sequence = Primer
gctcacaccatcaacaccgtt

SEQ ID NO: 114 - DNA, 21 bp, Artificial sequence
  Artificial Sequence = Primer
cgttgacttagtcaggataat

SEQ ID NO: 115 - DNA, 21 bp, Artificial sequence
  Artificial Sequence = Primer
gggcccactctaaccaaagaa

SEQ ID NO: 116 - DNA, 21 bp, Artificial sequence
  Artificial Sequence = Primer
cggtaattacctgaaactaaa

SEQ ID NO: 117 - DNA, 21 bp, Artificial sequence
  Artificial Sequence = Primer
cgtgtgggagccctgagcctt

SEQ ID NO: 118 - DNA, 20 bp, Artificial sequence
  Artificial Sequence = Primer
ccggcctggttgctagtatt

SEQ ID NO: 119 - DNA, 21 bp, Artificial sequence
  Artificial Sequence = Primer
catcagttgctagtgcgaatg

SEQ ID NO: 120 - DNA, 20 bp, Artificial sequence
  Artificial Sequence = Primer
cagcaaatgtcaaatacgtt

SEQ ID NO: 121 - DNA, 21 bp, Artificial sequence
  Artificial Sequence = Primer
cgacatcgacgatctatgact

SEQ ID NO: 122 - DNA, 21 bp, Artificial sequence
  Artificial Sequence = Primer
ccaagggatattgctgaaata

SEQ ID NO: 123 - DNA, 21 bp, Artificial sequence
  Artificial Sequence = Primer
catcagttgctagtgcgaatg

SEQ ID NO: 124 - DNA, 20 bp, Artificial sequence
  Artificial Sequence = Primer
cagcaaatgtcaaatacgtt

SEQ ID NO: 125 - DNA, 21 bp, Artificial sequence
  Artificial Sequence = Primer
cggagagtacgaccggcgctt

SEQ ID NO: 126 - DNA, 21 bp, Artificial sequence
  Artificial Sequence = Primer
gcacggctggctgctttcgtt

SEQ ID NO: 127 - DNA, 21 bp, Artificial sequence
  Artificial Sequence = Primer
gctgccattaatagcgcaact

SEQ ID NO: 128 - DNA, 21 bp, Artificial sequence
  Artificial Sequence = Primer
tattgttgttaccagccttgc

SEQ ID NO: 129 - DNA, 21 bp, Artificial sequence
  Artificial Sequence = Primer
gtaatgacggttaattctgtt

SEQ ID NO: 130 - DNA, 21 bp, Artificial sequence
  Artificial Sequence = Primer
gccgcatcaatagccttagaa

SEQ ID NO: 131 - DNA, 20 bp, Artificial sequence
  Artificial Sequence = Primer
cccataacggaacaactcat

SEQ ID NO: 132 - DNA, 22 bp, Artificial sequence
  Artificial Sequence = Primer
cagaatagaccaaacatctgca

SEQ ID NO: 133 - DNA, 21 bp, Artificial sequence
  Artificial Sequence = Primer
ggccactttcaatgttggtca

SEQ ID NO: 134 - DNA, 22 bp, Artificial sequence
  Artificial Sequence = Primer
cgactgcacctgttcctgatta

SEQ ID NO: 135 - DNA, 21 bp, Artificial sequence
  Artificial Sequence = Primer
tctgatatagtttatatgggt

SEQ ID NO: 136 - DNA, 21 bp, Artificial sequence
  Artificial Sequence = Primer
tcaaaccccactcttaattaa

SEQ ID NO: 137 - DNA, 21 bp, Artificial sequence
  Artificial Sequence = Primer
ttgcaaaagcaattttgcaac

SEQ ID NO: 138 - DNA, 21 bp, Artificial sequence
  Artificial Sequence = Primer
tgccgaacaatgttctctgca

SEQ ID NO: 139 - DNA, 21 bp, Artificial sequence
  Artificial Sequence = Primer
aattgttttaaaatctgttct

SEQ ID NO: 140 - DNA, 21 bp, Artificial sequence
  Artificial Sequence = Primer
tgagactgaaattacatttaa

SEQ ID NO: 141 - DNA, 21 bp, Artificial sequence
  Artificial Sequence = Primer
gaacaattcaaacagttcagt

SEQ ID NO: 142 - DNA, 21 bp, Artificial sequence
  Artificial Sequence = Primer
ttattcaaatcgcgcaatacc

SEQ ID NO: 143 - DNA, 20 bp, Artificial sequence
  Artificial Sequence = Primer
caaatacagtccgcgtacga

SEQ ID NO: 144 - DNA, 21 bp, Artificial sequence
  Artificial Sequence = Primer
ccattgttacctaaagagcgt

SEQ ID NO: 145 - DNA, 21 bp, Artificial sequence
  Artificial Sequence = Primer
ttggcagttacaggaatgcat

SEQ ID NO: 146 - DNA, 21 bp, Artificial sequence
  Artificial Sequence = Primer
aacagtgaaccatattttagt

SEQ ID NO: 147 - DNA, 20 bp, Artificial sequence
  Artificial Sequence = Primer
atgaggaacataatggcagg

SEQ ID NO: 148 - DNA, 20 bp, Artificial sequence
  Artificial Sequence = Primer
tctggtcaggtcgtcaatac

SEQ ID NO: 149 - DNA, 21 bp, Artificial sequence
  Artificial Sequence = Primer
ggtgatcgattattccgctga

SEQ ID NO: 150 - DNA, 21 bp, Artificial sequence
  Artificial Sequence = Primer
acgcctcatcggtcagcgcct

SEQ ID NO: 151 - DNA, 21 bp, Artificial sequence
  Artificial Sequence = Primer
tctgcacgtttaaaattattg

SEQ ID NO: 152 - DNA, 21 bp, Artificial sequence
  Artificial Sequence = Primer
gtttctccgtcagaatcaagc

SEQ ID NO: 153 - DNA, 21 bp, Artificial sequence
  Artificial Sequence = Primer
ctaccgtagcgggcgatggta

SEQ ID NO: 154 - DNA, 21 bp, Artificial sequence
  Artificial Sequence = Primer
cagcgcctgtccgtgttcggc

SEQ ID NO: 155 - DNA, 21 bp, Artificial sequence
  Artificial Sequence = Primer
ccctgaccttgggtgttgcga

SEQ ID NO: 156 - DNA, 20 bp, Artificial sequence
  Artificial Sequence = Primer
gtactgaactttaaaggtgg

SEQ ID NO: 157 - DNA, 20 bp, Artificial sequence
  Artificial Sequence = Primer
aagaaatcaatattatttat

SEQ ID NO: 158 - DNA, 18 bp, Artificial sequence
  Artificial Sequence = Primer
aatagcacccggtacaag

SEQ ID NO: 159 - DNA, 20 bp, Artificial sequence
  Artificial Sequence = Primer
gcgaaggaatttaccttaga

SEQ ID NO: 160 - DNA, 20 bp, Artificial sequence
  Artificial Sequence = Primer
cagctgtcacagtaacaaac

SEQ ID NO: 161 - DNA, 20 bp, Artificial sequence
  Artificial Sequence = Primer
cttgaacatatatctcaggg

SEQ ID NO: 162 - DNA, 21 bp, Artificial sequence
  Artificial Sequence = Primer
acaggagcagtttcagacagt

SEQ ID NO: 163 - DNA, 21 bp, Artificial sequence
  Artificial Sequence = Primer
ggtggagtatacaaaatataa

SEQ ID NO: 164 - DNA, 21 bp, Artificial sequence
  Artificial Sequence = Primer
atgacaggcattagttttaat

SEQ ID NO: 165 - DNA, 20 bp, Artificial sequence
  Artificial Sequence = Primer
ttctgttaatgcaatggcgg

SEQ ID NO: 166 - DNA, 21 bp, Artificial sequence
  Artificial Sequence = Primer
ttcagcaaatccggagcctga

SEQ ID NO: 167 - DNA, 20 bp, Artificial sequence
  Artificial Sequence = Primer
gaagaagatgtttatagcgg

SEQ ID NO: 168 - DNA, 21 bp, Artificial sequence
  Artificial Sequence = Primer
actgcaggtattagatatgat

SEQ ID NO: 169 - DNA, 22 bp, Artificial sequence
  Artificial Sequence = Primer
attggtgccggtgttactgctg

SEQ ID NO: 170 - DNA, 20 bp, Artificial sequence
  Artificial Sequence = Primer
ctcccatacctaaacgcaat

SEQ ID NO: 171 - DNA, 21 bp, Artificial sequence
  Artificial Sequence = Primer
attggtgttgccgtcaccgct

SEQ ID NO: 172 - DNA, 18 bp, Artificial sequence
  Artificial Sequence = Primer
acgccatgacatgggagg

SEQ ID NO: 173 - DNA, 21 bp, Artificial sequence
  Artificial Sequence = Primer
attggtgctggtgtaacgact

SEQ ID NO: 174 - DNA, 18 bp, Artificial sequence
  Artificial Sequence = Primer
attgcgtttaggtatggg

SEQ ID NO: 175 - DNA, 21 bp, Artificial sequence
  Artificial Sequence = Primer
ctactgttcccgagtagtgtg

SEQ ID NO: 176 - DNA, 21 bp, Artificial sequence
  Artificial Sequence = Primer
ggtgccgtccggaatcggcgt